(19) United States
(12) Patent Application Publication
(10) Pub. No.: US 2022/0073616 A1
(43) Pub. Date: Mar. 10, 2022

(54) METHODS OF ADMINISTERING ANTI-TIM-3 ANTIBODIES

(71) Applicant: Merck Patent GmbH, Darmstadt (DE)

(72) Inventors: Mary Ruisi, Andover, MA (US); Rinat Zaynagetdinov, Bedford, MA (US); Dong Zhang, Westwood, MA (US); Xinyan Zhao, Bedford, MA (US); Qi An, Nashua, NH (US); David Nannemann, Woburn, MA (US); Vanita Sood, Somerville, MA (US); Christel Iffland, Arlington, MA (US)

(21) Appl. No.: 17/245,476

(22) Filed: Apr. 30, 2021

Related U.S. Application Data
(63) Continuation of application No. PCT/US2019/059556, filed on Nov. 1, 2019.
(60) Provisional application No. 62/754,378, filed on Nov. 1, 2018.

(51) Int. Cl. C07K 16/28; A61P 35/00; C07K 14/495

(52) U.S. Cl. CPC C07K 16/2827; A61K 2039/545; C07K 14/495; A61P 35/00

(57) ABSTRACT

The invention is based, in part, upon the discovery of a family of antibodies that specifically bind human T Cell Immunoglobulin and Mucin Domain-3 (TIM-3). More specifically, this invention relates to a method of treating cancer by administering an anti-TIM-3 antibody in combination with an anti-PD-L1/TGFβ Trap fusion protein. When administered to a human cancer patient or an animal model, the antibodies inhibit or reduce tumor growth in the human patient or animal model.

### CROSS-REFERENCE TO RELATED APPLICATIONS

This application claims the benefit of and priority to U.S. Provisional Patent Application No. 62/754,378, filed Nov. 1, 2018, the entire disclosures of which are incorporated by reference herein.

### FIELD OF THE INVENTION

The field of the invention is molecular biology, immunology and oncology. More particularly, the field is therapeutic antibodies.

### BACKGROUND

Following the approval of Yervoy® (ipilimumab, Bristol-Myers Squibb) for melanoma in 2011, immune checkpoint inhibitors have become a promising class of molecules for therapeutic development (for example, those targeting PD-1, PD-L1, and CTLA-4). Several large companies developing immune checkpoint inhibitor drugs include Bristol-Myers Squibb, Merck & Co., Roche, AstraZeneca and many others. The developmental strategies and investment in immunotherapy, together with compelling clinical efficacy have led to several new approvals of anti-PD(L)-1 drugs: Keytruda® (pembrolizumab, Merck & Co.), Opdivo® (nivolumab, Bristol-Myers Squibb), Tecentriq® (atezolizumab, Roche), Bavencio® (avelumab, EMD Serono), and Imfinzi® (durvalumab, AstraZeneca).

PD-1/PD-L1 checkpoint inhibitors, with their compelling clinical efficacy and safety profiles, have built a solid foundation for combination immunotherapy approaches. These strategies include combining PD-1 pathway inhibitors with inhibitors of other immune checkpoint proteins expressed on T-cells. One such checkpoint protein is T Cell Immunoglobulin and Mucin Domain-3 (TIM-3), also known as Hepatitis A Virus Cellular Receptor 2 (HAVCR2).

Tim-3 was first identified as a molecule selectively expressed on IFN-g-producing CD4+ T helper 1 (Th1) and CD8+ T cytotoxic 1 (Tc1) T cells (Monney et al. (2002) NATURE 415(6871):536-41). TIM-3 is also expressed on the surface of many immune cell types, including certain subsets of T cells such as FOXP3⁺CD4⁺ T regulatory cells (Tregs), natural killer (NK) cells, monocytes, and tumor-associated dendritic cells (TADCs) (Clayton et al. (2014) J. IMMUNOL. 192(2):782-791; Jones et al. (2008) J. EXP. MED. 205(12):2763-79; Hastings et al. (2009) EUR. J. IMMUNOL 39(9):2492-2501; Seki et al. (2008) CLIN IMMUNOL 127(1):78-88; Ju et al. (2010) J HEPATOL 52(3):322-329; Anderson et al. (2007) SCIENCE 318(5853):1141-1143; Baitsch et al. (2012) PLOS ONE 7(2):e30852; Ndhlovu et al. (2012) BLOOD 119(16):3734-3743). Putative ligands of TIM-3 have been reported, including phosphatidylserine (PtdSer; Nakayama et al., (2009) BLOOD 113(16):3821-30), galectin-9 (Gal-9) (Zhu et al. (2005) NAT IMMUNOL 6(12):1245-52), high-mobility group protein 1 (HMGB1) (Chiba et al. (2012) NAT IMMUNOL 13(9):832-42), and carcinoembryonic antigen cell adhesion molecule 1 (CEACAM1) (Huang et al. (2015) NATURE 517(7534):386-90).

Studies suggest that TIM-3 regulates various aspects of the immune response. The interaction of TIM-3 and its ligand galectin-9 (Gal-9) induces cell death. The in vivo blockade of this interaction exacerbated autoimmunity and abrogated tolerance in experimental models, suggesting that TIM-3/Gal-9 interaction negatively regulates immune responses (Zhu et al. (2005), supra; Kanzaki et al. (2012) ENDOCRINOLOGY 153(2):612-620). The inhibition of TIM-3 also enhanced the pathological severity in in vivo experimental autoimmune encephalomyelitis (Monney et al. (2002) NATURE 415:536-541; Das, et al. (2017) IMMUNOL REV 276(1):97-111). In studies using materials from human patients with multiple sclerosis (Koguchi et al. (2006) J EXP MED 203(6):1413-1418), Crohn's disease (CD) (Morimoto et al. (2011) SCAND J GASTROENTEROL 46(6):701-709) and rheumatoid arthritis (RA) (Liu et al. (2010) CLIN IMMUNOL 137(2):288-295; Li et al. (2014) PLoS ONE 9(2):e85784), the observation that Tim-3 expression level on T cells is inversely correlated with autoimmune disease progression suggests an immunosuppressive role of TIM-3 on T-cells. In addition to the effect on T-cells, TIM-3/Gal-9 interaction leads to antimicrobial activity by promoting macrophage clearance of intracellular pathogens (Sakuishi et al. (2011) TRENDS IMMUNOL 32(8):345-349), and TIM-3 may also promote clearance of apoptotic cells by binding phosphatidyl serine through its unique binding cleft (DeKruyff et al. (2010) J IMMUNOL 184(4):1918-1930).

Tim-3 is considered a potential candidate for cancer immunotherapy, in part, because it is upregulated in tumor-infiltrating lymphocytes including Foxp3+CD4+ Treg and exhausted CD8+ T cells, two key immune cell populations that constitute immunosuppression in tumor environment of many human cancers (McMahan et al. (2010) J. CLIN. INVEST. 120(12):4546-4557; Jin et al. (2010) PROC NATL ACAD SCI USA 107(33):14733-8; Golden-Mason et al. (2009) J VIROL 83(18):9122-9130; Fourcade et al. (2010) J EXP MED 207(10):2175-86; Sakuishi et al. (2010) J EXP MED 207(10):2187-94; Zhou et al. (2011) BLOOD 117(17):4501-4510; Ngiow et al., (2011) CANCER RES. 71(10):3540-51, Yan, et al. (2013) PLoS ONE 8(3):e58006). The molecular mechanism of T cell dysregulation is hypothesized to begin with the interaction of Tim-3 on CD8+ T cells and its ligand galectin-9 on tumor cells, which results in the phosphorylation of the Tim-3 cytoplasmic tail at tyrosines 256 and 263, leading to the release of HLA-B-associated transcript 3 (Bat3) and catalytically active lymphocyte-specific protein tyrosine kinase (Lck) from the Tim-3 cytoplasmic tail. The dissociation of Bat3 and Lck from Tim-3 leads to the accumulation of inactive phosphorylated Lck, which may account for the observed T cell dysfunction (Rangachari, et al. (2012) NAT MED 18(9):1394-400).

Further, intratumoral Tim-3+FoxP3+ Treg cells appear to express high amounts of Treg effector molecules (IL-10, perforin, and granzymes). Tim-3+ Tregs are thought to promote the development of a dysfunctional phenotype in CD8+ tumor infiltrating lymphocytes (TILs) in tumor environment (Sakuishi, et al. (2013) ONCOIMMUNOLOGY 2(4):e23849). Tim-3 has also been reported to have effects in the myeloid compartment. T-cell expression of Tim-3 has been shown to promote CD11b+Gr-1+ myeloid-derived suppressor cells (MDSC) in a galectin-9-dependent manner (Dardalhon, et al. (2010) J IMMUNOL 185(3):1383-92). Furthermore, as Tim-3 is specifically upregulated on tumor-associated dendritic cells (TADC), it is able to interfere with the sensing of DNA released by cells undergoing necrotic cell death. Tim-3 binds to high mobility group protein 1 (HMGB1), thereby prevents HMGB1 from binding to DNA released from dying cells and mediating delivery to innate cells via receptor for advanced glycation end (RAGE) products and/or Toll-like receptors (TLR) 2 and 4 pathways. Tim-3 binding to HMGB 1 dampens activation of the innate immune response in tumor tissue (Chiba, et al. (2012), supra). Taken together, these data suggest that Tim-3 can further suppress antitumor T-cell responses by T-cell extrinsic mechanisms involving myeloid cells and different Tim-3/ligand interactions.

The synergy of Tim-3/PD-1 co-blockade in inhibiting tumor growth in preclinical mouse tumor models suggests that the co-blockade modulates the functional phenotype of dysfunctional CD8+T cells and/or Tregs (Sakuishi et al. (2010), supra; Ngiow et al. (2011), supra). Indeed, besides in vivo co-blockade with PD(L)-1, co-blockade with many other check-point inhibitors enhances anti-tumor immunity and suppresses tumor growth in many preclinical tumor models (Dardalhon et al. (2010), supra; Nglow et al., CANCER RES 2011; Chiba et al. (2012), supra; Baghdadi et al., CANCER IMMUNOL IMMUNOTHER 2013; Kurtulus et al. (2015) J CLIN INVEST 125(11):4053-62; Huang et al. (2015), supra; Sakuishi et al. (2010), supra; Jing et al. (2015) J IMMUNOTHER CANCER 3:2; Zhou et al. (2011), supra; Komohara et al., CANCER IMMUNOLOGY RES., 2015).

Despite the success of checkpoint inhibitors such as Yervoy®, Keytruda® and Opdivo® and others, only a fraction of the patients experience durable clinical responses to these therapies. Some tumor types have shown little response to anti-CTLA-4 or anti-PD-1/PD-L1 monotherapies in clinical trials. These include prostate, colorectal, and pancreatic cancers. Accordingly, for these nonresponsive diseases and for the majority who are non-responders within responsive tumor types, there is a need for improved anti-tumor therapies.

### SUMMARY OF THE INVENTION

The invention relates in part to methods of treating cancer using a family of antibodies that specifically bind human T Cell Immunoglobulin and Mucin Domain-3 (TIM-3). The antibodies contain TIM-3 binding sites based on the complementarity determining regions (CDRs) of the antibodies. The antibodies can be used as therapeutic agents alone or in combination with other therapeutic agents, such as other immune checkpoint inhibitors. When used as therapeutic agents, the antibodies can be optimized, e.g., affinity-matured, to improve biochemical properties (e.g., affinity and/or specificity), to improve biophysical properties (e.g., aggregation, stability, precipitation, and/or non-specific interactions), and/or to reduce or eliminate immunogenicity, when administered to a human patient.

The antibodies described herein inhibit TIM-3 from binding to TIM-3 ligands, e.g., galectin-9, phosphatidylserine (PtdSer), and carcinoembryonic antigen-related cell adhesion molecule 1 (CEACAM1). The disclosed antibodies can be used to inhibit the proliferation of tumor cells in vitro or in vivo. When administered to a human cancer patient or an animal model, the antibodies inhibit or reduce tumor growth in the human patient or animal model.

Accordingly, in one aspect, the disclosure relates to a method of treating cancer in a mammal, the method comprising administering an effective amount of an anti-TIM-3 antibody and a second therapeutic agent to the mammal in need thereof.

In another aspect, the disclosure relates to an anti-TIM-3 antibody for use in a method of treating cancer in a mammal, the method comprising administering an effective amount of an anti-TIM-3 antibody and a second therapeutic agent to the mammal in need thereof.

In another aspect, the disclosure relates to the use of an anti-TIM-3 antibody in the manufacture of a medicament for use in a method of treating cancer in a mammal, the method comprising administering an effective amount of an anti-TIM-3 antibody and a second therapeutic agent to the mammal in need thereof.

In certain embodiments, the anti-TIM-3 antibody is administered in an amount of from about 0.1 mg/kg to about 100 mg/kg. In certain embodiments, the anti-TIM-3 antibody is administered as a flat (fixed) dose of from about 5 mg to about 3500 mg.

In certain embodiments, the second therapeutic agent is an anti-PD-L1/TGFβ Trap fusion protein. In certain embodiments, the anti-PD-L1/TGFβ Trap fusion protein comprises:

(a) a heavy chain comprising an CDR_{H1}, an CDR_{H2}, and an CDR-13, having at least 80% overall sequence identity to SYIMM (SEQ ID NO: 78), SIYPSGGITFYADTVKG (SEQ ID NO: 79), and IKLGTVTTVDY (SEQ ID NO: 80), respectively, and

(b) a light chain comprising an CDR_{L1}, an CDR_{L2}, and an CDR_{L3}, having at least 80% overall sequence identity to TGTSSDVGGYNYVS (SEQ ID NO: 81), DVSNRPS (SEQ ID NO: 82), and SSYTSSSTRV (SEQ ID NO: 83), respectively.

In certain embodiments, the anti-PD-L1/TGFβ Trap fusion protein is bintrafusp. In certain embodiments, the anti-PD-L1/TGFβ Trap fusion protein is bintrafusp alfa.

In certain embodiments, the anti-PD-L1/TGFβ Trap fusion protein is administered in a flat (fixed) dose of from about 800 mg to about 2600 mg. In certain embodiments, the anti-PD-L1/TGFβ Trap fusion protein is administered in a flat (fixed) dose of about 1200 mg. In certain embodiments, the anti-PD-L1/TGFβ Trap fusion protein is administered in a flat (fixed) dose of about 2400 mg. In certain embodiments, the anti-TIM-3 antibody and/or the anti-PD-L1/TGFβ Trap fusion protein is administered every two weeks. In certain embodiments, the anti-TIM-3 antibody and/or the anti-PD-L1/TGFβ Trap fusion protein is administered every three weeks.

In certain embodiments, the cancer is selected from the group consisting of diffuse large B-cell lymphoma, renal cell carcinoma (RCC), non-small cell lung carcinoma (NSCLC), squamous cell carcinoma of the head and neck (SCCHN), triple negative breast cancer (TNBC) or gastric/stomach adenocarcinoma (STAD).

In certain embodiments, the mammal is a human.

In certain embodiments, the anti-TIM-3 antibody comprises

(i) an immunoglobulin heavy chain variable region comprising a CDR_{H1} comprising the amino acid sequence of SEQ ID NO: 1, a CDR_{H2} comprising the amino acid sequence of SEQ ID NO: 2, and a CDR_{H3} comprising the amino acid sequence of SEQ ID NO: 3; and

(ii) an immunoglobulin light chain variable region comprising a CDR_{L1} comprising the amino acid sequence of SEQ ID NO: 4, a CDR_{L2} comprising the amino acid sequence of SEQ ID NO: 5, and a CDR_{L3} comprising the amino acid sequence of SEQ ID NO: 6.

In certain embodiments, the anti-TIM-3 antibody comprises an immunoglobulin heavy chain variable region selected from the group consisting of SEQ ID NO: 53, SEQ ID NO: 24, SEQ ID NO: 55, SEQ ID NO: 34, and an immunoglobulin light chain variable region selected from the group consisting of SEQ ID NO: 52, SEQ ID NO: 54, SEQ ID NO: 23 and SEQ ID NO: 33.

In certain embodiments, the anti-TIM-3 antibody comprises an immunoglobulin heavy chain variable region comprising the amino acid sequence of SEQ ID NO: 24, and an immunoglobulin light chain variable region comprising the amino acid sequence of SEQ ID NO: 23.

In certain embodiments, the anti-TIM-3 antibody comprises an immunoglobulin heavy chain and an immunoglobulin light chain selected from the group consisting of:

(a) an immunoglobulin heavy chain comprising the amino acid sequence of SEQ ID NO: 22, and an immunoglobulin light chain comprising the amino acid sequence of SEQ ID NO: 21; and

(b) an immunoglobulin heavy chain comprising the amino acid sequence of SEQ ID NO: 32, and an immunoglobulin light chain comprising the amino acid sequence of SEQ ID NO: 31.

In certain embodiments, the anti-TIM-3 antibody has a KD of 9.2 nM or lower, as measured by surface plasmon resonance.

In certain embodiments, the anti-TIM-3 antibody competes for binding to the galectin-9, the PtdSer, and/or the carcinoembryonic antigen cell adhesion-related molecule 1 (CEACAM1) binding site on human TIM-3 with an antibody comprising:

(A) (i) an immunoglobulin heavy chain variable region comprising a CDR_{H1} comprising the amino acid sequence of SEQ ID NO: 1, a CDR_{H2} comprising the amino acid sequence of SEQ ID NO: 2, and a CDR_{H3} comprising the amino acid sequence of SEQ ID NO: 3; and

(ii) an immunoglobulin light chain variable region comprising a CDR_{L1} comprising the amino acid sequence of SEQ ID NO: 4, a CDR_{L2} comprising the amino acid sequence of SEQ ID NO: 5, and a CDR_{L3} comprising the amino acid sequence of SEQ ID NO: 6; and/or

(B) an immunoglobulin heavy chain variable region selected from the group consisting of SEQ ID NO: 53, SEQ ID NO: 24, SEQ ID NO: 55, SEQ ID NO: 34, and an immunoglobulin light chain variable region selected from the group consisting of SEQ ID NO: 52, SEQ ID NO: 54, SEQ ID NO: 23 and SEQ ID NO: 33; and/or

(C) an immunoglobulin heavy chain variable region comprising the amino acid sequence of SEQ ID NO: 24, and an immunoglobulin light chain variable region comprising the amino acid sequence of SEQ ID NO: 23; and/or

(D) an immunoglobulin heavy chain and an immunoglobulin light chain selected from the group consisting of:

(a) an immunoglobulin heavy chain comprising the amino acid sequence of SEQ ID NO: 22, and an immunoglobulin light chain comprising the amino acid sequence of SEQ ID NO: 21; and

(b) an immunoglobulin heavy chain comprising the amino acid sequence of SEQ ID NO: 32, and an immunoglobulin light chain comprising the amino acid sequence of SEQ ID NO: 31.

In certain embodiments, the anti-TIME-3 antibody binds to the same epitope on a human TIM-3 protein as an antibody as described herein, wherein the epitope includes P59, F61, E62, and D120 of the human TIM-3 protein.

These and other aspects and advantages of the invention will become apparent upon consideration of the following figures, detailed description, and claims. As used herein, “including” means without limitation, and examples cited are non-limiting.

### BRIEF DESCRIPTION OF THE DRAWINGS

The foregoing and other objects, features and advantages of the invention will become apparent from the following description of preferred embodiments, as illustrated in the accompanying drawings. Like referenced elements identify common features in the corresponding drawings. The drawings are not necessarily to scale, with emphasis instead being placed on illustrating the principles of the present invention, in which:

FIGS. 1A-D shows the crystal structure of human TIM-3 in complex with M6903. FIG. 1A shows an overview of the Fab portion of M6903 (upper structure) bound to TIM-3 shown as a surface representation. Extensive contacts made on TIM-3 (bottom structure) are shown as the lighter portion of TIM-3. FIG. 1B shows the epitope hotspot residues of TIM-3 (e.g., P59 and F61 and E62). FIG. 1C shows the polar head group of ptdSer (light-colored sticks) and the coordinating calcium ion (sphere) have been modeled into the structure of M6903-bound TIM-3 by superposition with the structure of murine TIM-3 (DeKruyff et al. (2010), supra). The binding site of ptdSer coincides with the placement of Y59 (group of spheres) of the heavy chain from M6903. Hydrogen bonds from D120 on TIM-3 to ptdSer or M6903, respectively, are shown as dotted lines. FIG. 1D shows the polar interactions of M6903 with the CEACAM-1 binding residues of TIM-3 are shown with dashed lines.

FIG. 2 depicts a model of the crystal structure of TIM-3 with an anti-TIM-3 antibody 3903E11 (VL1.3,VH1.2) epitope map showing the P59, F61, E62, I114, N119, and K122 residues which reside on the face of one beta sheet of the immunoglobulin fold.

FIG. 3 provides a graph showing that target occupancy of anti-TIM-3 antibody M6903 on CD14⁺ monocytes increased with increased concentrations of anti-TIM-3. Serial dilutions of anti-TIM-3 antibody 3903E11 (VL1.3,VH1.2) IgG2h (FN-AQ,322A)-delK (M6903) were incubated with fresh human whole blood for 1 hour. The unoccupied TIM-3 on CD14+ cells was measured by flow cytometry with anti-TIM-3 (2E2)-APC, which competes with the anti-TIM-3 antibody for TIM-3 binding. The average EC50 across all 10 donors was 111.1±85.6 ng/ml. The graph shows 4 representative donors (KP46233, KP46231, KP46315, and KP46318) out of the 10 total donors.

FIG. 4 provides a graph showing that M6903 efficiently blocked the interaction of rhTIM-3 and PtdSer on apoptotic Jurkat cells. Prior to flow cytometry analysis, apoptosis was induced in Jurkat cells via treatment with Staurosporine (2 μg/mL, 18 hrs), leading to surface expression of a TIM-3 ligand, PtdSer. Binding of rhTIM-3-Fc PtdSer on the surface of apoptotic Jurkat cells was evaluated via flow cytometry by measuring the MFI of rhTIM-3-Fc after pre-incubation with serial dilutions of M6903 or an anti-HEL IgG2h isotype control. While the isotype control had no effect, M6903 blocked the interaction of rhTIM-3 and PtdSer with an IC₅₀ of 4.438±3.115 nM (0.666±0.467 μg/ml). A nonlinear fit line was applied to the graph using a Sigmoid dose-response equation.

FIGS. 5A and 5B depict graphs showing M6903 increased CEF antigen specific T cell activation in a dose-dependent manner. The combination of M6903 and bintrafusp further enhanced this activation. PBMCs were treated with 40 μg/ml CEF viral peptide pool for (A) 6 days or (B) 4 days in the presence of M6903. In FIG. 5A, M6903 dose-dependently enhanced T cell activation compared to isotype control in a CEF assay as measured by IFN-γ production, with an EC50 of 1±1.3 μg/mL, calculated from multiple experiments. Non-linear regression analysis was performed and mean and SD are presented. In FIG. 5B, serial dilutions of M6903 were combined with either 10 μg/mL isotype control or bintrafusp alfa. The combination with bintrafusp alfa led to a further increase in IFN-γ production. Mean and SD are presented (p<0.05).

FIGS. 6A and 6B provide graphs showing M6903 dose-dependently enhancement of allo-antigen specific T cell activation. T cell activation was evaluated in an allogenic one-way MLR assay by measuring IFN-γ in the supernatant of co-cultured irradiated Daudi cells and human T cells after 2 days of treatment. In FIG. 6A, co-cultured cells were treated with serial dilutions of M6903 or isotype control. M6903 dose-dependently enhanced allo-antigen specific T cell activation, with an EC50 of 116±117 ng/mL. In FIG. 6B, co-cultured cells were treated with serial dilutions of M6903 combined with 10 μg/mL of isotype control or bintrafusp alfa. The combination of M6903 with bintrafusp alfa further enhanced T cell activation. Nonlinear regression analysis was performed and mean±SD are presented for both graphs.

FIG. 7 provides a graph demonstrating that M6903 exhibits enhanced activity in combination with bintrafusp in a superantigen SEB assay. Human PBMCs were treated with 100 ng/mL SEB along with 10 mg/mL M6903 (or isotype control) either alone or in combination with bintrafusp alfa for 9 days. Cells were then washed once with medium and re-stimulated with SEB and the same antibodies for another 2 days. Supernatants were harvested and IFN-γ was measured by IFN-γ ELISA. M6903 and bintrafusp alfa both increased IFN-γ production in SEB-stimulated T cells, and the effect was enhanced by combining M6903 with bintrafusp alfa.

FIG. 8 depicts the results of a CEF antigen-specific T cell assay using M6903, anti-PdtSer, and anti-Ga19. PBMCs were treated with 40 μg/ml CEF viral peptide pool for 5 days in the presence of the antibody or antibodies indicated. The combination of anti-Gal-9 and anti-PtdSer had similar activity as M6903 alone, suggesting that blocking both Gal-9 and PtdSer may be required for anti-TIM-3 activity (compare data outlined by boxes).

FIGS. 9A-9B depict a quantitative analysis of TIM-3 expression measured via THC in 12 tumor TMAs stained with anti-TIM-3 antibody. In FIG. 9A, the plot is ordered by median expression and in FIG. 9B, the plot is ordered by average expression following the removal of outliers.

FIG. 10 depicts mIF staining of 8 tumor tissues to identify immune cells expressing TIM-3 in the tumor microenvironment (TME). CD3 and CD68 were used as markers for lymphocytes and macrophages, respectively. The percentage of TIM-3⁺CD3⁺ lymphocytes and TIM-3⁺CD68⁺ macrophages was quantified across the tumor TMAs using mIF analysis.

FIG. 11 depicts TIM-3 expression in an NSCLC cohort using flow cytometry analysis. Within live CD3+ cells, expression of TIM-3 was observed to be highest on CD8+ T cells, followed by CD4+ T cells and Tregs. Each dot represents an individual sample. Lines represent the median value for each immune subset.

FIGS. 12A-B demonstrate that M6903 and bintrafusp, as monotherapies or combination, decreased MC38 tumor volume in B-huTIM-3 KI mice. B-huTIM-3 KI mice were inoculated with MC38 (1×10⁶ cells) s.c. in the flank and then treated with isotype control (20 mg/kg), M6903 (10 mg/kg), bintrafusp alfa (24 mg/kg) or M6903+bintrafusp alfa. FIG. 12A shows average tumor volumes with SEM and FIG. 12B shows individual tumor volumes.

FIG. 13 shows a dose escalation scheme in which, following a 28 day screening period, the subject is administered the M6903 escalation dose by IV infusion every two weeks. The two-week M6903 monotherapy lead-in period is followed by administration of the M6903 escalation dose in combination with 1200 mg of bintrafusp alfa (“BFA”) by IV infusion every two weeks.

### DETAILED DESCRIPTION

The anti-TIM-3 antibodies disclosed herein are based on the antigen binding sites of certain monoclonal antibodies that have been selected on the basis of binding and neutralizing the activity of human T Cell Immunoglobulin and Mucin Domain-3 (TIM-3). The antibodies contain immunoglobulin variable region CDR sequences that define a binding site for TIM-3.

In view of the neutralizing activity of these antibodies, they are useful for inhibiting the growth and/or proliferation of certain types of cancer cells. When used as a therapeutic agent, the antibodies can be optimized, e.g., affinity-matured, to improve biochemical properties and/or biophysical properties, and/or to reduce or eliminate immunogenicity when administered to a human patient. Various features and aspects of the invention are discussed in more detail below.

As used herein, unless otherwise indicated, the term “antibody” means an intact antibody (e.g., an intact monoclonal antibody) or antigen-binding fragment of an antibody, including an intact antibody or antigen-binding fragment of an antibody (e.g., a phage display antibody including a fully human antibody, a semisynthetic antibody or a fully synthetic antibody) that has been optimized, engineered or chemically conjugated. Examples of antibodies that have been optimized are affinity-matured antibodies. Examples of antibodies that have been engineered are Fc optimized antibodies, antibody fusion proteins and multispecific antibodies (e.g., bispecific antibodies). Examples of antigen-binding fragments include Fab, Fab′, F(ab)₂, Fv, single chain antibodies (e.g., scFv), minibodies and diabodies. An antibody conjugated to a toxin moiety is an example of a chemically conjugated antibody. Antibody fusion proteins include, for example, an antibody genetically fused to a soluble ligand such as a cytokine, or to an extracellular domain of a cellular receptor protein.

I. Antibodies that Bind Human TIM-3

The antibodies disclosed herein comprise: (a) an immunoglobulin heavy chain variable region comprising a CDR_{H1}, a CDR_{H2}, and a CDR_{H3} and (b) an immunoglobulin light chain variable region comprising a CDR_{L1}, a CDR_{L2}, and a CDR_{L3}, wherein the heavy chain variable region and the light chain variable region together define a single binding site for binding TIM-3 protein.

In some embodiments, the antibody comprises: (a) an immunoglobulin heavy chain variable region comprising a CDR_{H1}, a CDR_{H2}, and a CDR_{H3} and (b) an immunoglobulin light chain variable region, wherein the heavy chain variable region and the light chain variable region together define a single binding site for binding TIM-3. A CDR_{H1} comprises the amino acid sequence of SEQ ID NO: 1; a CDR_{H2} comprises the amino acid sequence of SEQ ID NO: 2; and a CDR_{H3} comprises the amino acid sequence of SEQ ID NO: 3. The CDR_{H1}, CDR_{H2}, and CDR_{H3} sequences are interposed between immunoglobulin FR sequences (SEQ ID NO: 7, SEQ ID NO:8, SEQ ID NO: 9, and SEQ ID NO:10).

In some embodiments, the antibody comprises (a) an immunoglobulin light chain variable region comprising a CDR_{L1}, a CDR_{L2}, and a CDR_{L3}, and (b) an immunoglobulin heavy chain variable region, wherein the IgG light chain variable region and the IgG heavy chain variable region together define a single binding site for binding TIM-3. A CDR_{L1} comprises the amino acid sequence of SEQ ID NO: 4; a CDR_{L2} comprises the amino acid sequence of SEQ ID NO: 5; and a CDR_{L3} comprises the amino acid sequence of SEQ ID NO: 6. The CDR_{L1}, CDR_{L2}, and CDR_{L3} sequences are interposed between immunoglobulin FR sequences (SEQ ID NO: 11, SEQ ID NO: 12, SEQ ID NO: 13, and SEQ ID NO: 14).

In some embodiments, the antibody comprises: (a) an immunoglobulin heavy chain variable region comprising a CDR_{H1}, a CDR_{H2}, and a CDR_{H3} and (b) an immunoglobulin light chain variable region comprising a CDR_{L1}, a CDR_{L2}, and a CDR_{L3}, wherein the heavy chain variable region and the light chain variable region together define a single binding site for binding TIM-3. The CDR_{H1} is the amino acid sequence of SEQ ID NO: 1; the CDR_{H2} is the amino acid sequence of SEQ ID NO: 2; and the CDR_{H3} is the amino acid sequence of SEQ ID NO: 3. The CDR_{L1} is the amino acid sequence of SEQ ID NO: 4; the CDR_{L2} is the amino acid sequence of SEQ ID NO: 5; and the CDR_{L3} is the amino acid sequence of SEQ ID NO: 6.

In other embodiments, the antibodies disclosed herein comprise an immunoglobulin heavy chain variable region and an immunoglobulin light chain variable region. In some embodiments, the antibody comprises an immunoglobulin heavy chain variable region comprising an amino acid sequence selected from the group consisting of SEQ ID NO: 53, SEQ ID NO: 24, SEQ ID NO: 55, and SEQ ID NO: 34; and an immunoglobulin light chain variable region.

In other embodiments, the antibody comprises an immunoglobulin light chain variable region selected from the group consisting of SEQ ID NO: 52, SEQ ID NO: 54, SEQ ID NO: 23 and SEQ ID NO: 33; and an immunoglobulin heavy chain variable region.

In some embodiments, the antibody comprises an immunoglobulin heavy chain variable region comprising an amino acid sequence selected from the group consisting of SEQ ID NO: 53, SEQ ID NO: 24, SEQ ID NO: 55, and SEQ ID NO: 34; and an immunoglobulin light chain variable region selected from the group consisting of SEQ ID NO: 52, SEQ ID NO: 54, SEQ ID NO: 23 and SEQ ID NO: 33.

In some embodiments, the antibody comprises an immunoglobulin heavy chain variable region comprising the amino acid sequence of SEQ ID NO: 24, and an immunoglobulin light chain variable region comprising the amino acid sequence of SEQ ID NO: 23.

In certain embodiments, the antibodies disclosed herein comprise an immunoglobulin heavy chain and an immunoglobulin light chain. In some embodiments, the antibody comprises an immunoglobulin heavy chain selected from the group consisting of SEQ ID NO: 16, SEQ ID NO: 18, SEQ ID NO: 20, SEQ ID NO: 22, and SEQ ID NO: 32; and an immunoglobulin light chain.

In other embodiments, the antibody comprises an immunoglobulin light chain selected from the group consisting of SEQ ID NO: 15, SEQ ID NO: 17, SEQ ID NO: 19, SEQ ID NO: 21, and SEQ ID NO: 31; and an immunoglobulin heavy chain.

In some embodiments, the antibody comprises (i) an immunoglobulin heavy chain comprising an amino acid sequence selected from the group consisting of SEQ ID NO: 16, SEQ ID NO: 18, SEQ ID NO: 20, SEQ ID NO: 22, and SEQ ID NO: 32; and (ii) an immunoglobulin light chain selected from the group consisting of SEQ ID NO: 15, SEQ ID NO: 17, SEQ ID NO: 19, SEQ ID NO: 21, and SEQ ID NO: 31.

In some embodiments, the antibody comprises an immunoglobulin heavy chain comprising the amino acid sequence of SEQ ID NO: 22 and an immunoglobulin light chain comprising the amino acid sequence of SEQ ID NO: 21.

In certain embodiments, an isolated antibody that binds TIM-3 comprises an immunoglobulin heavy chain variable region comprising an amino acid sequence that is at least 70%, 75%, 80%, 85%, 90%, 95%, 98%, or 99% identical to the entire variable region or the framework region sequence of SEQ ID NO: 16, SEQ ID NO: 18, SEQ ID NO: 20, SEQ ID NO: 22, or SEQ ID NO: 32. In certain embodiments, an isolated antibody that binds TIM-3 comprises an immunoglobulin heavy chain variable region comprising a CDR_{H1} comprising the amino acid sequence of SEQ ID NO: 1; a CDR_{H2} comprising the amino acid sequence of SEQ ID NO: 2; and a CDR_{H3} comprising the amino acid sequence of SEQ ID NO: 3; and an amino acid sequence that is at least 70%, 75%, 80%, 85%, 90%, 95%, 98%, or 99% identical to the entire variable region or the framework region sequence of SEQ ID NO: 16, SEQ ID NO: 18, SEQ ID NO: 20, SEQ ID NO: 22, or SEQ ID NO: 32.

In certain embodiments, an isolated antibody that binds TIM-3 comprises an immunoglobulin light chain variable region comprising an amino acid sequence that is at least 70%, 75%, 80%, 85%, 90%, 95%, 98%, or 99% identical to the entire variable region or the framework region sequence of SEQ ID NO: 15, SEQ ID NO: 17, SEQ ID NO: 19, SEQ ID NO: 21, or SEQ ID NO: 31. In certain embodiments, an isolated antibody that binds TIM-3 comprises an immunoglobulin light chain variable region comprising a CDR_{L1} comprising the amino acid sequence of SEQ ID NO: 4; a CDR_{L2} comprising the amino acid sequence of SEQ ID NO: 5; and a CDR_{L3} comprising the amino acid sequence of SEQ ID NO: 6; and an amino acid sequence that is at least 70%, 75%, 80%, 85%, 90%, 95%, 98%, or 99% identical to the entire variable region or the framework region sequence of SEQ ID NO: 15, SEQ ID NO: 17, SEQ ID NO: 19, SEQ ID NO: 21, or SEQ ID NO: 31.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and -Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In each of the foregoing embodiments, it is contemplated herein that immunoglobulin heavy chain variable region sequences and/or light chain variable region sequences that together bind TIM-3 may contain amino acid alterations (e.g., at least 1, 2, 3, 4, 5, or 10 amino acid substitutions, deletions, or additions) in the framework regions of the heavy and/or light chain variable regions. In certain embodiments, the amino acid alterations are conservative substitutions. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

In certain embodiments, the antibody binds TIM-3 with a K_{D} of 20 nM, 15 nM, 10 nM, 9 nM, 8 nM, 7 nM, 6 nM, 5 nM, 4 nM, 3 nM, 2 nM, 1 nM or lower. Unless otherwise specified, K_{D} values are determined by surface plasmon resonance. For example, surface plasmon resonance can be measured using a GE Healthcare Biacore 4000 instrument as follows. Goat anti-human Fc antibody (Jackson Immunoresearch Laboratories #109-005-098) is immobilized on BIAcore carboxymethylated dextran CM5 chip using direct coupling to free amino groups following the procedure described by the manufacturer. Antibodies are captured on the CMS biosensor chip to achieve approximately 200 response units (RU). Binding measurements are performed using the running HBS-EP+ buffer. A 2-fold dilution series starting at 100 nM of anti-TIM-3 antibodies are injected at a flow rate of 30 μl/min at 25° C. Association rates (kon, M-1s-1) and dissociation rates (koff, s-1) are calculated using a simple 1:1 Langmuir binding model (Biacore 4000 Evaluation Software). The equilibrium dissociation constant (KD, M) is calculated as the ratio of koff/kon.

In some embodiments, monoclonal antibodies bind to the same epitope on TIM-3 as any of the anti-TIM-3 antibodies disclosed herein (e.g., M6903). In some embodiments, monoclonal antibodies compete for binding to TIM-3 with any of the anti-TIM-3 antibodies disclosed herein. For example, monoclonal antibodies may compete for binding to the galectin-9 binding domain of TIM-3 with an anti-TIM-3 antibody described herein. In another example, monoclonal antibodies may compete for binding to the PtdSer binding domain of TIM-3 with an anti-TIM-3 antibody described herein. In another example, monoclonal antibodies may compete for binding to the CEACAM1 binding domain of TIM-3 with an anti-TIM-3 antibody described herein. In a further example, monoclonal antibodies may compete for binding to the galectin-9 binding domain and the PtdSer binding domain of TIM-3 with an anti-TIM-3 antibody described herein. In another example, monoclonal antibodies may compete for binding to the galectin-9 binding domain and the CEACAM1 binding domain of TIM-3 with an anti-TIM-3 antibody described herein. In another example, monoclonal antibodies may compete for binding to the PtdSer binding domain and the CEACAM1 binding domain of TIM-3 with an anti-TIM-3 antibody described herein. In another example, monoclonal antibodies may compete for binding to the galectin-9 binding domain, the PtdSer binding domain, and the CEACAM1 binding domain of TIM-3 with an anti-TIM-3 antibody described herein.

Competition assays for determining whether an antibody binds to the same epitope as an anti-TIM-3 antibody described herein, or competes for binding with galectin-9, PtdSer, and/or CEACAM1 with an anti-TIM-3 antibody described herein are known in the art. Exemplary competition assays include immunoassays (e.g., ELISA assays, RIA assays), BIAcore analysis, biolayer interferometry and flow cytometry.

Typically, a competition assay involves the use of an antigen (e.g., a TIM-3 protein or fragment thereof) bound to a solid surface or expressed on a cell surface, a test TIM-3-binding antibody and a reference antibody (e.g., antibody M6903). The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (i.e., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

In an exemplary competition assay, a reference TIM-3 antibody (e.g., antibody M6903) is biotinylated using commercially available reagents. The biotinylated reference antibody is mixed with serial dilutions of the test antibody or unlabeled reference antibody (self-competition control) resulting in a mixture of various molar ratios (e.g., 1×, 5×, 10×, 20× or 100×) of test antibody (or unlabeled reference antibody) to labeled reference antibody. The antibody mixture is added to a TIM-3 (e.g., TIM-3 extracellular domain) polypeptide coated-ELISA plate. The plate is then washed and HRP (horseradish peroxidase)-strepavidin is added to the plate as the detection reagent. The amount of labeled reference antibody bound to the target antigen is detected following addition of a chromogenic substrate (e.g., TMB (3,3′,5,5′-tetramethylbenzidine) or ABTS (2,2″-azino-di-(3-ethylbenzthiazoline-6-sulfonate)), which are well-known in the art. Optical density readings (OD units) are measured using a SpectraMax M2 spectrometer (Molecular Devices). OD units corresponding to zero percent inhibition are determined from wells without any competing antibody. OD units corresponding to 100% inhibition, i.e., the assay background are determined from wells without any labeled reference antibody or test antibody. Percent inhibition of labeled reference antibody to TIM-3 by the test antibody (or the unlabeled reference antibody) at each concentration is calculated as follows: % inhibition=(1−(OD units−100% inhibition)/(0% inhibition−100% inhibition))*100. Persons skilled in the art will appreciate that the competition assay can be performed using various detection systems well-known in the art.

A competition assay may be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

### II. Anti-PD-L1/TGFβ Trap Fusion Proteins

The anti-TIM-3 antibodies described herein can be administered in combination with any anti-PD-L1/TGFβ Trap known in the art. “Anti-PD-L1/TGFβ Trap” refers to a fusion molecule comprising 1) an antibody or antigen-binding fragment thereof that is capable of binding PD-L1 and antagonizing the interaction between PD-1 and PD-L1 and 2) a TGFβRII or fragment of TGFβRII that is capable of binding TGFβ and antagonizing the interaction between TGFβ and TGFβRII.

In one embodiment, the anti-PD-L1/TGFβ Trap comprises an anti-PD-L1 antibody known in the art. Anti-PD-L1 antibodies are commercially available, for example, the 29E2A3 antibody (Biolegend, Cat. No. 329701). Antibodies can be monoclonal, chimeric, humanized, or human. Antibody fragments include Fab, F(ab′)2, scFv and Fv fragments, which are described in further detail below.

Exemplary anti-PD-L1 antibodies are described in PCT Publication WO 2013/079174, which describes avelumab. These antibodies can include a heavy chain variable region polypeptide including a CDR_{H1}, CDR_{H2}, and CDR_{H3} sequence, where:

(a) the CDR_{H1} sequence is X₁YX₂MX₃ (SEQ ID NO: 58);

(b) the CDR_{H2} sequence is SIYPSGGX₄TFYADX₅VKG (SEQ ID NO: 59);

(c) the CDR_{H3} sequence is IKLGTVTTVX₆Y (SEQ ID NO: 60);

further where: X₁ is K, R, T, Q, G, A, W, M, I, or S; X₂ is V, R, K, L, M, or I; X₃ is H, T, N, Q, A, V, Y, W, F, or M; X₄ is F or I; X₅ is S or T; X₆ is E or D.

In a one embodiment, X₁ is M, I, or S; X₂ is R, K, L, M, or I; X₃ is F or M; X₄ is F or I; X₅ is S or T; X₆ is E or D.

In another embodiment X₁ is M, I, or S; X₂ is L, M, or I; X₃ is F or M; X₄ is I; X₅ is S or T; X₆ is D.

In still another embodiment, X₁ is S; X₂ is I; X₃ is M; X₄ is I; X₅ is T; X₆ is D.

In another aspect, the polypeptide further includes variable region heavy chain framework (FR) sequences juxtaposed between the CDRs according to the formula: (HC-FR1)-(CDR_{H1})-(HC-FR2)-(CDR_{H2})-(HC-FR3)-(CDR_{H3})-(HC-FR4).

In yet another aspect, the framework sequences are derived from human consensus framework sequences or human germline framework sequences.

In a still further aspect, at least one of the framework sequences is the following:

HC-FR1 is
(SEQ ID NO: 61)
EVQLLESGGGLVQPGGSLRLSCAASGFTFS;
HC-FR2 is
(SEQ ID NO: 62)
WVRQAPGKGLEWVS;
HC-FR3 is
(SEQ ID NO: 63)
RFTISRDNSKNTLYLQMNSLRAEDTAVYYCAR;
HC-FR4 is
(SEQ ID NO: 64)
WGQGTLVTVSS.

In a still further aspect, the heavy chain polypeptide is further combined with a variable region light chain including a CDR_{L1}, CDR_{L2}, and CDR_{L3}, where:

(a) the CDR_{L1} sequence is TGTX₇X₈DVGX₉YNYVS (SEQ ID NO: 65);

(b) the CDR_{L2} sequence is X₁₀VX₁₁X₁₂RPS (SEQ ID NO: 66);

(c) the CDR_{L3} sequence is SSX₁₃TX₁₄X₁₅X₁₆X₁₇RV (SEQ ID NO: 67);

further where: X₇ is N or S; X₈ is T, R, or S; X₉ is A or G; X₁₀ is E or D; X₁₁ is I, N or S; X₁₂ is D, H or N; X₁₃ is F or Y; X₁₄ is N or S; X₁₅ is R, T or S; X₁₆ is G or S; X₁₇ is I or T.

In another embodiment, X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In still another embodiment, X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a still further aspect, the light chain further includes variable region light chain framework sequences juxtaposed between the CDRs according to the formula: (LC-CDR_{L1})-(LC-FR2)-(CDR_{L2})-(LC-FR3)-(CDR_{L3})-(LC-FR4).

In a still further aspect, the light chain framework sequences are derived from human consensus framework sequences or human germline framework sequences.

In a still further aspect, the light chain framework sequences are lambda light chain sequences.

In a still further aspect, at least one of the framework sequence is the following:

LC-FR1 is
(SEQ ID NO: 68)
QSALTQPASVSGSPGQSITISC;
LC-FR2 is
(SEQ ID NO: 69)
WYQQHPGKAPKLMIY;
LC-FR3 is
(SEQ ID NO: 70)
GVSNRFSGSKSGNTASLTISGLQAEDEADYYC;
LC-FR4 is
(SEQ ID NO: 71)
FGTGTKVTVL.

In another embodiment, the invention provides an anti-PD-L1 antibody or antigen binding fragment including a heavy chain and a light chain variable region sequence, where:

(a) the heavy chain includes a CDR_{H1}, CDR_{H2}, and CDR_{H3}, wherein further: (i) the CDR_{H1} sequence is X₁YX₂MX³ (SEQ ID NO: 72); (ii) the CDR_{H2} sequence is SIYPSGGX₄TFYADX₅VKG (SEQ ID NO: 73); (iii) the CDR_{H3} sequence is IKLGTVTTVX₆Y (SEQ ID NO: 74), and;

(b) the light chain includes a CDR_{L1}, CDR_{L2}, and CDR_{L3}, wherein further: (iv) the CDR_{L1} sequence is TGTX₇X₈DVGX₉YNYVS (SEQ ID NO: 75); (v) the CDR_{L2} sequence is X₁₀VX₁₁X₁₂RPS (SEQ ID NO: 76); (vi) the CDR_{L3} sequence is SSX₁₃TX₁₄X₁₅X₁₆X₁₇RV (SEQ ID NO: 77); wherein: X₁ is K, R, T, Q, G, A, W, M, I, or S; X₂ is V, R, K, L, M, or I; X₃ is H, T, N, Q, A, V, Y, W, F, or M; X₄ is F or I; X₅ is S or T; X₆ is E or D; X₇ is N or S; X₈ is T, R, or S; X₉ is A or G; X₁₀ is E or D; X₁₁ is I, N, or S; X₁₂ is D, H, or N; X₁₃ is F or Y; X₁₄ is N or S; X₁₅ is R, T, or S; X₁₆ is G or S; X₁₇ is I or T.

In one embodiment, X₁ is M, I, or S; X₂ is R, K, L, M, or I; X₃ is F or M; X₄ is F or I; X₅ is S or T; X₆ is E or D; X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In another embodiment, X₁ is M, I, or S; X₂ is L, M, or I; X₃ is F or M; X₄ is I; X₅ is S or T; X₆ is D; X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In still another embodiment, X₁ is S; X₂ is I; X₃ is M; X₄ is I; X₅ is T; X₆ is D; X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a further aspect, the heavy chain variable region includes one or more framework sequences juxtaposed between the CDRs as: (HC-FR1)-(CDR_{H1})-(HC-FR2)-(CDR_{H2})-(HC-FR3)-(CDR_{H3})-(HC-FR4), and the light chain variable regions include one or more framework sequences juxtaposed between the CDRs as: (LC-FR1 MCDR_{L1})-(LC-FR2)-(CDR_{L2})-(LC-FR3)-(CDR_{L3})-(LC-FR4).

In a still further aspect, the framework sequences are derived from human consensus framework sequences or human germline sequences.

In a still further aspect, one or more of the heavy chain framework sequences is the following:

HC-FR1 is
(SEQ ID NO: 61)
EVQLLESGGGLVQPGGSLRLSCAASGFTFS;
HC-FR2 is
(SEQ ID NO: 62)
WVRQAPGKGLEWVS;
HC-FR3 is
(SEQ ID NO: 63)
RFTISRDNSKNTLYLQMNSLRAEDTAVYYCAR;
HC-FR4 is
(SEQ ID NO: 64)
WGQGTLVTVSS.

In a still further aspect, the light chain framework sequences are lambda light chain sequences.

In a still further aspect, one or more of the light chain framework sequences is the following:

LC-FR1 is
(SEQ ID NO: 68)
QSALTQPASVSGSPGQSITISC;
LC-FR2 is
(SEQ ID NO: 69)
WYQQHPGKAPKLMIY;
LC-FR3 is
(SEQ ID NO: 70)
GVSNRFSGSKSGNTASLTISGLQAEDEADYYC;
LC-FR4 is
(SEQ ID NO: 71)
FGTGTKVTVL.

In a still further aspect, the heavy chain variable region polypeptide, antibody, or antibody fragment further includes at least a C_{H}1 domain.

In a more specific aspect, the heavy chain variable region polypeptide, antibody, or antibody fragment further includes a C_{H}1, a C_{H}2, and a C_{H}3 domain.

In a still further aspect, the variable region light chain, antibody, or antibody fragment further includes a C_{L} domain.

In a still further aspect, the antibody further includes a C_{H}1, a C_{H}2, a C_{H}3, and a C_{L} domain.

In a still further specific aspect, the antibody further includes a human or murine constant region.

In a still further aspect, the human constant region is selected from the group consisting of IgG1, IgG2, IgG2, IgG3, IgG4.

In a still further specific aspect, the human or murine constant region is IgG1.

In yet another embodiment, the invention features an anti-PD-L1 antibody including a heavy chain and a light chain variable region sequence, where:

(a) the heavy chain includes a CDR_{H1}, a CDR_{H2}, and a CDR_{H3}, having at least 80% overall sequence identity to SYIMM (SEQ ID NO: 78), SIYPSGGITFYADTVKG (SEQ ID NO: 79), and IKLGTVTTVDY (SEQ ID NO: 80), respectively, and

(b) the light chain includes a CDR_{L1}, a CDR_{L2}, and a CDR_{L3}, having at least 80% overall sequence identity to TGTSSDVGGYNYVS (SEQ ID NO: 81), DVSNRPS (SEQ ID NO: 82), and SSYTSSSTRV (SEQ ID NO: 83), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In yet another embodiment, the invention features an anti-PD-L1 antibody including a heavy chain and a light chain variable region sequence, where:

(a) the heavy chain includes a CDR_{H1,} a CDR_{H2}, and a CDR_{H3}, having at least 80% overall sequence identity to MYMMM (SEQ ID NO: 84), SIYPSGGITFYADSVKG (SEQ ID NO: 85), and IKLGTVTTVDY (SEQ ID NO: 80), respectively, and

(b) the light chain includes a CDR_{L1}, a CDR_{L2}, and a CDR_{L3}, having at least 80% overall sequence identity to TGTSSDVGAYNYVS (SEQ ID NO: 86), DVSNRPS (SEQ ID NO: 82), and SSYTSSSTRV (SEQ ID NO: 83), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In a still further aspect, in the antibody or antibody fragment according to the invention, as compared to the sequences of CDR_{H1}, CDR_{H2}, and CDR_{H3}, at least those amino acids remain unchanged that are highlighted by underlining as follows:

(a) in
(SEQ ID NO: 78)
CDRHISYIMM,
(b) in
(SEQ ID NO: 79)
CDRH2SIYPSGGITFYADTVKG,
(c) in
(SEQ ID NO: 80)
CDRH3IKLGTVTTVDY;

and further where, as compared to the sequences of CDR_{L1}, CDR_{L2}, and CDR_{L3} at least those amino acids remain unchanged that are highlighted by underlining as follows:

(a) CDRL1
(SEQ ID NO: 81)
TGTSSDVGGYNYVS
(b) CDR-L2
(SEQ ID NO: 82)
DVSNRPS
(c) CDRL3
(SEQ ID NO: 83)
SSYTSSSTRV.

In another aspect, the heavy chain variable region includes one or more framework sequences juxtaposed between the CDRs as: (HC-FR1)-(CDR_{H1})-(HC-FR2)-(CDR_{H2})-(HC-FR3)-(CDR_{H3})-(HC-FR4), and the light chain variable regions include one or more framework sequences juxtaposed between the CDRs as: (LC-FR1)-(CDR_{L1})-(LC-FR2)-(CDR_{L2})-(LC-FR3)-(CDR_{L3})-(LC-FR4).

In yet another aspect, the framework sequences are derived from human germline sequences.

In a still further aspect, one or more of the heavy chain framework sequences is the following:

HC-FR1 is
(SEQ ID NO: 61)
EVQLLESGGGLVQPGGSLRLSCAASGFTFS;
HC-FR2 is
(SEQ ID NO: 62)
WVRQAPGKGLEWVS;
HC-FR3 is
(SEQ ID NO: 63)
RFTISRDNSKNTLYLQMNSLRAEDTAVYYCAR;
HC-FR4 is
(SEQ ID NO: 64)
WGQGTLVTVSS.

In a still further aspect, the light chain framework sequences are derived from a lambda light chain sequence.

In a still further aspect, one or more of the light chain framework sequences is the following:

LC-FRI is
(SEQ ID NO: 68)
QSALTQPASVSGSPGQSITISC;
LC-FR2 is
(SEQ ID NO: 69)
WYQQHPGKAPKLMIY;
LC-FR3 is
(SEQ ID NO: 70)
GVSNRFSGSKSGNTASLTISGLQAEDEADYYC;
LC-FR4 is
(SEQ ID NO: 71)
FGTGTKVTVL.

In a still further specific aspect, the antibody further includes a human or murine constant region.

In a still further aspect, the human constant region is selected from the group consisting of IgG1, IgG2, IgG2, IgG3, IgG4.

In a still further embodiment, the invention features an anti-PD-L1 antibody including a heavy chain and a light chain variable region sequence, where:

(a) the heavy chain sequence has at least 85% sequence identity to the heavy chain sequence: EVQLLESGGGLVQPGGSLRLSCAASGFTFSSYIMMVWRQAPGKGLEWVSSIYPSGGITF YADWKGRFTISRDNSKNTLYLQMNSLRAEDTAVYYCARIKLGTVTTVDYWGQGTLVT VSS (SEQ ID NO: 87), and

(b) the light chain sequence has at least 85% sequence identity to the light chain sequence: QSALTQPASVSGSPGQSITISCTGTSSDVGGYNYVSWYQQHPGKAPKLMIYDVSN RPSGVSNRFSGSKSGNTASLTISGLQAEDEADYYCSSYTSSSTRVFGTGTKVTVL (SEQ ID NO: 88).

In a specific aspect, the sequence identity is 86%, 87%, 88%, 89%, 90%, 91%, 92%, 93%, 94%, 95%, 96%, 97%, 98%, 99%, or 100%.

In a still further embodiment, the invention provides for an anti-PD-L1 antibody including a heavy chain and a light chain variable region sequence, where:

(a) the heavy chain sequence has at least 85% sequence identity to the heavy chain sequence: EVQLLESGGGLVQPGGSLRLSCAASGFTFSMYMMMWVRQAPGKGLEVWSSIYPSGGIT FYADSVKGRFTISRDNSKNTLYLQMNSLRAEDTAIYYCARIKLGTVTTVDYWG QGTLVTVSS (SEQ ID NO: 89), and

(b) the light chain sequence has at least 85% sequence identity to the light chain sequence: QSALTQPASVSGSPGQSMSCTGTSSDVGAYNYVSWYQQHPGKAPKLMIYDVSNR PSGVSNRFSGSKSGNTASLTISGLQAEDEADYYCSSYTSSSTRVFGTGTKVTVL (SEQ ID NO: 90).

In a specific aspect, the sequence identity is 86%, 87%, 88%, 89%, 90%, 91%, 92%, 93%, 94%, 95%, 96%, 97%, 98%, 99% or 100%.

In a particular embodiment, anti-PD-L1/TGFβ Trap is one of the fusion molecules disclosed in WO 2015/118175 or WO 2018/205985. For instance, anti-PD-LUTGFβ Trap may comprise the light chains and heavy chains of SEQ ID NO: 1 and SEQ ID NO: 3 of WO 2015/118175, respectively. In another embodiment, anti-PD-L1/TGFβ Trap is one of the constructs listed in Table 2 of WO 2018/205985, such as construct 9 or 15 thereof. In other embodiments, the antibody having the heavy chain sequence of SEQ ID NO: 11 and the light chain sequence of SEQ ID NO: 12 of WO 2018/205985 is fused via a linking sequence (G4S)xG, wherein x is 4-5, to the TGFβRII extracellular domain sequence of SEQ ID NO: 14 or SEQ ID NO: 15 of WO 2018/205985.

In one embodiment, the anti-PD-L1/TGFβ Trap is a protein having the amino acid sequence of bintrafusp alfa, as described in International Patent Publication WO 2015/118175 and as reflected by the amino acid sequence given by CAS Registry Number 1918149-01-5. Bintrafusp alfa comprises a light chain that is identical to the light chain of an anti-PD-L1 antibody (SEQ ID NO: 91). Bintrafusp alfa further comprises a fusion polypeptide having the sequence corresponding SEQ ID NO: 93, composed of the heavy chain of an anti-PD-L1 antibody (SEQ ID NO: 92), wherein the C-terminal lysine residue of heavy chain was mutated to alanine, genetically fused to via a flexible (Gly4Ser)4Gly linker (SEQ ID NO: 97) to the N-terminus of the soluble TGFβ Receptor 11 (SEQ ID NO: 96). Bintrafusp alfa is encoded by SEQ ID NO: 94 (DNA encoding the anti-PD-L1 light chain) and SEQ ID NO: 95 (DNA encoding the anti-PD-L1/TGFβ Receptor II).

In one embodiment, the anti-PD-L1/TGFβ Trap is bintrafusp alfa, a protein having the amino acid sequence of bintrafusp alpha and also a glycosylation form that results from the protein being produced in CHO cells, wherein the heavy chain is glycosylated at Asn-300, Asn-518, Asn-542, and Asn-602 (i.e., of SEQ ID NO: 93). (See, WHO Drug Information, Vol. 32, No. 2, 2018, p. 293.)

Peptide sequence of the secreted LC
of anti-PD-L1
(SEQ ID NO: 91)
QSALTQPASVSGSPGQSITISCTGTSSDVGGYNYV
SWYQQHPGKAPKLMIYDVSNRPSGVSNRFSGSKSG
NTASLTISGLQAEDEADYYCSSYTSSSTRVFGTGT
KVTVLGQPKANPTVTLFPPSSEELQANKATLVCLI
SDFYPGAVTVAWKADGSPVKAGVETTKPSKQSNNK
YAASSYLSLTPEQWKSHRSYSCQVTHEGSTVEKTV
APTECS
Peptide sequence of the secreted H
chain of anti-PDL1
(SEQ ID NO: 92)
EVQLLESGGGLVQPGGSLRLSCAASGFTFSSYIMM
WVRQAPGKGLEWVSSIYPSGGITFYADTVKGRFTI
SRDNSKNTLYLQMNSLRAEDTAVYYCARIKLGTVT
TVDYWGQGTLVTVSSASTKGPSVFPLAPSSKSTSG
GTAALGCLVKDYFPEPVTVSWNSGALTSGVHTFPA
VLQSSGLYSLSSVVTVPSSSLGTQTYICNVNHKPS
NTKVDKRVEPKSCDKTHTCPPCPAPELLGGPSVFL
FPPKPKDTLMISRTPEVTCVVVDVSHEDPEVKFNW
YVDGVEVHNAKTKPREEQYNSTYRVVSVLTVLHQD
WLNGKEYKCKVSNKALPAPIEKTISKAKGQPREPQ
VYTLPPSREEMTKNQVSLTCLVKGFYPSDIAVEWE
SNGQPENNYKTTPPVLDSDGSFFLYSKLTVDKSRW
QQGNVFSCSVMHEALHNHYTQKSLSLSPGK
Peptide sequence of the secreted H
chain of anti-PDL1/TGFβ Trap
(SEQ ID NO: 93)
EVQLLESGGGLVQPGGSLRLSCAASGFTFSSYIMM
WVRQAPGKGLEWVSSIYPSGGIIFYADTVKGRFTI
SRDNSKNTLYLQMNSLRAEDTAVYYCARIKLGTVT
TVDYWGQGTLVTVSSASTKGPSVFPLAPSSKSTSG
GTAALGCLVKDYFPEPVTVSWNSGALTSGVHTFPA
VLQSSGLYSLSSVVTVPSSSLGTQTYICNVNHKPS
NTKVDKRVEPKSCDKTHTCPPCPAPELLGGPSVFL
FPPKPKDTLMISRTPEVTCVVVDVSHEDPEVKFNW
YVDGVEVHNAKTKPREEQYNSTYRVVSVLTVLHQD
WLNGKEYKCKVSNKALPAPIEKTISKAKGQPREPQ
VYTLPPSREEMTKNQVSLTCLVKGFYPSDIAVEWE
SNGQPENNYKTTPPVLDSDGSFFLYSKLTVDKSRW
QQGNVFSCSVMHEALHNHYTQKSLSLSPGAGGGGS
GGGGSGGGGSGGGGSGIPPHVQKSVNNDMIVTDNN
GAVKFPQLCKFCDVRFSTCDNQKSCMSNCSITSIC
EKPQEVCVAVWRKNDENITLETVCHDPKLPYHDFI
LEDAASPKCIMKEKKKPGETFFMCSCSSDECNDNI
IFSEEYNTSNPD

DNA sequence from the translation initiation codon to the translation stop codon of the anti-PD-L1 lambda light chain (the leader sequence preceding the VL is the signal peptide from urokinase plasminogen activator)

(SEQ ID NO: 94)
atgagqgccctgctggctagactgctgctgtgcgtg
ctggtcgtgtccgacagcaagggcCAGTCCGCCCT
GACCCAGCCTGCCTCCGTGTCTGGCTCCCCTGGCC
AGTCCATCACCATCAGCTGCACCGGCACCTCCAGC
GACGTGGGCGGCTACAACTACGTGTCCTGGTATCA
GCAGCACCCCGGCAAGGCCCCCAAGCTGATGATCT
ACGACGTGTCCAACCGGCCCTCCGGCGTGTCCAAC
AGATTCTCCGGCTCCAAGTCCGGCAACACCGCCTC
CCTGACCATCAGCGGACTGCAGGCAGAGGACGAGG
CCGACTACTACTGCTCCTCCTACACCTCCTCCAGC
ACCAGAGTGTTCGGCACCGGCACAAAAGTGACCGT
GCTGggccagcccaaggccaacccaaccgtgacac
tgttccccccatcctccgaggaactgcaggccaac
aaggccaccctggtctgcctgatctcagatttcta
tccaggcgccgtgaccgtggcctggaaggctgatg
gctccccagtgaaggccggcgtggaaaccaccaag
ccctccaagcagtccaacaacaaatacgccgcctc
ctcctacctgtccctgacccccgagcagtggaagt
cccaccggtcctacagctgccaggtcacacacgag
ggctccaccgtggaaaagaccgtcgcccccaccga
gtgctcaTGA

DNA sequence from the translation initiation codon to the translation stop codon (mVK SP leader: small underlined; VH: capitals; IgG1m3 with K to A mutation: small letters; (G4S)x4-G linker: bold capital letters; TGFβRII: bold underlined small letters; two stop codons: bold underlined capital letters)

(SEQ ID NO: 95)
atggaaacagacaccctgctgctgtgggtgctgct
gctgtgggtgcccggctccacaggcGAGGTGCAGC
TGCTGGAATCCGGCGGAGGACTGGTGCAGCCTGGC
GGCTCCCTGAGACTGTCTTGCGCCGCCTCCGGCTT
CACCTTCTCCAGCTACATCATGATGTGGGTGCGAC
AGGCCCCTGGCAAGGGCCTGGAATGGGTGTCCTCC
ATCTACCCCTCCGGCGGCATCACCTTCTACGCCGA
CACCGTGAAGGGCCGGTTCACCATCTCCCGGGACA
ACTCCAAGAACACCCTGTACCTGCAGATGAACTCC
CTGCGGGCCGAGGACACCGCCGTGTACTACTGCGC
CCGGATCAAGCTGGGCACCGTGACCACCGTGGACT
ACTGGGGCCAGGGCACCCTGGTGACAGTGTCCTCC
gctagcaccaagggcccatcggtcttccccctggc
accctcctccaagagcacctctgggggcacagcgg
ccctgggctgcctggtcaaggactacttccccgaa
ccggtgacggtgtcgtggaactcaggcgccctgac
cagcggcgtgcacaccttcccggctgtcctacagt
cctcaggactctactccctcagcagcgtggtgacc
gtgccctccagcagcttgggcacccagacctacat
ctgcaacgtgaatcacaagcccagcaacaccaagg
tggacaagagagttgagcccaaatcttgtgacaaa
actcacacatgcccaccgtgcccagcacctgaact
cctggggggaccgtcagtcttcctcttccccccaa
aacccaaggacaccctcatgatctcccggacccct
gaggtcacatgcgtggtggtggacgtgagccacga
agaccctgaggtcaagttcaactggtacgtggacg
gcgtggaggtgcataatgccaagacaaagccgcgg
gaggagcagtacaacagcacgtaccgtgtggtcag
cgtcctcaccgtcctgcaccaggactggctgaatg
gcaaggagtacaagtgcaaggtctccaacaaagcc
ctcccagcccccatcgagaaaaccatctccaaagc
caaagggcagccccgagaaccacaggtgtacaccc
tgcccccatcccgggaggagatgaccaagaaccag
gtcagcctgacctgcctggtcaaaggcttctatcc
cagcgacatcgccgtggagtgggagagcaatgggc
agccggagaacaactacaagaccacgcctcccgtg
ctggactccgacggctccttcttcctctatagcaa
gctcaccgtggacaagagcaggtggcagcagggga
acgtcttctcatgctccgtgatgcatgaggctctg
cacaaccactacacgcagaagagcctctccctgtc
cccgggtgctGGCGGCGGAGGAAGCGGAGGAGGTG
GCAGCGGTGGCGGTGGCTCCGGCGGAGGTGGCTCC
GGAatccctccccacgtgcagaagtccgtgaacaa
cgacatgatcgtgaccgacaacaacggcgccgtga
agttccctcagctgtgcaagttctgcgacgtgagg
ttcagcacctgcgacaaccagaagtcctgcatgag
caactgcagcatcacaagcatctgcgagaagcccc
aggaggtgtgtgtggccgtgtggaggaagaacgac
gaaaacatcaccctcgagaccgtgtgccatgaccc
caagctgccctaccacgacttcatcctggaagacg
ccgcctcccccaagtgcatcatgaaggagaagaa
gaagcccggcgagaccttcttcatgtgcagctgca
gcagcgacgagtgcaatgacaacatcatctttag
cgaggagtacaacaccagcaaccccgacTGATAA

### A Human TGFβRII Isoform B Extracellular Domain Polypeptide

(SEQ ID NO: 96)
IPPHVQKSVNNDMIVTDNNGAVKFPQLCKFCDVRF
STCDNQKSCMSNCSITSICEKPQEVCVAVWRKNDE
NITLETVCHDPKLPYHDFILEDAASPKCIMKEKKK
PGETFFMCSCSSDECNDNIIFSEEYNTSNPD
(G1y₄Ser)₄G1y linker
(SEQ ID NO: 97)
GGGGSGGGGSGGGGSGGGGSG

Anti-PD-L1/TGFβ Trap molecules useful in the present invention may comprise sequences having at least 85%, at least 90%, at least 95%, at least 96%, at least 97%, at least 98%, or at least 99% sequence identity to any one of SEQ ID NOs: 91-96, as described above.

In some embodiments, the anti-PD-L1/TGFβ Trap is an anti-PD-L1/TGFβ Trap molecule disclosed in WO 2018/205985. For example, the anti-PD-L1/TGFβ Trap is one of the constructs listed in Table 2 of WO 2018/205985, such as construct 9 or 15 thereof.

In other embodiments, anti-PD-L1/TGFβ Trap is a heterotetramer, consisting of two polypeptides each having the light chain sequence corresponding to SEQ ID NO: 12 of WO 2018/205985 and two fusion polypeptides each having the heavy chain sequence corresponding to SEQ ID NO: 11 of WO 2018/205985 fused via a linker sequence (G4S)xG (wherein x can be 4 or 5) (SEQ ID NO: 117) to the TGFβRII extracellular domain sequence corresponding to SEQ ID NO: 14 (wherein “x” of the linker sequence is 4) or SEQ ID NO: 15 (wherein “x” of the linker sequence is 5) of WO 2018/205985.

In certain embodiments, an anti-PD-L1/TGFβ Trap molecule includes a first and a second polypeptide. The first polypeptide includes: (a) at least a variable region of a heavy chain of an antibody that binds to human protein Programmed Death Ligand 1 (PD-L1); and (b) human Transforming Growth Factor Receptor II (TGFβRII), or a fragment thereof, capable of binding Transforming Growth Factor β (TGFβ) (e.g., a soluble fragment). The second polypeptide includes at least a variable region of a light chain of an antibody that binds PD-L1, in which the heavy chain of the first polypeptide and the light chain of the second polypeptide, when combined, form an antigen binding site that binds PD-L1 (e.g., any of the antibodies or antibody fragments described herein). In certain embodiments, the anti-PD-L1/TGFβ Trap molecule is a heterotetramer, comprising the two immunoglobulin light chains of anti-PD-L1, and two heavy chains comprising the heavy chain of anti-PD-L1 genetically fused via a flexible glycine-serine linker (e.g., (G4S)xG (wherein x can be 4 or 5) (SEQ ID NO: 117)) to the extracellular domain of the human TGFβRII.

A Truncated Human TGFβRII Isoform B
Extracellular Domain Polypeptide
SEQ ID NO: 104
GAVKFPQLCKFCDVRFSTCDNQKSCMSNCSITSIC
EKPQEVCVAVWRKNDENITLETVCHDPKLPYHDFI
LEDAASPKCIMKEKKKPGETFFMCSCSSDECNDNI
IFSEEYNTSNPD
(identical to SEQ ID NO: 14
in WO 2018/205985)
A Truncated Human TGFβRII Isoform B
Extracellular Domain Polypeptide
SEQ ID NO: 105
VKFPQLCKFCDVRFSTCDNQKSCMSNCSITSICEK
PQEVCVAVWRKNDENITLETVCHDPKLPYHDFILE
DAASPKCIMKEKKKPGETFFMCSCSSDECNDNIIF
SEEYNTSNPD
(identical to SEQ ID NO: 15
in WO 2018/205985)
A Truncated Human TGFPβII Isoform B
Extracellular Domain Polypeptide
SEQ ID NO: 106
VTDNNGAVKFPQLCKFCDVRFSTCDNQKSCMSNCS
ITSICEKPQEVCVAVWRKNDENITLETVCHDPKLP
YHDFILEDAASPKCIMKEKKKPGETFFMCSCSSDE
CNDNIIFSEEYNTSNPD
A Truncated Human TGFPβII Isoform B
Extracellular Domain Polypeptide
SEQ ID NO: 107
LCKFCDVRFSTCDNQKSCMSNCSITSICEKPQEVC
VAVWRKNDENITLETVCHDPKLPYHDFILEDAASP
KCIMKEKKKPGETFFMCSCSSDECNDNIIFSEEYN
TSNPD
A Mutated Human TGFβRII Isoform B
Extracellular Domain Polypeptide
SEQ ID NO: 108
VTDNAGAVKFPQLCKFCDVRFSTCDNQKSCMSNCS
ITSICEKPQEVCVAVWRKNDENITLETVCHDPKLP
YHDFILEDAASPKCIMKEKKKPGETFFMCSCSSDE
CNDNIIFSEEYNTSNPD
Polypeptide sequence of the heavy
chain variable region of anti-PD-L1
antibody
SEQ ID NO: 109
QVQLQESGPGLVKPSQTLSLTCTVSGGSISNDYWT
WIRQHPGKGLEYIGYISYTGSTYYNPSLKSRVTIS
RDTSKNQFSLKLSSVTAADTAVYYCARSGGWLAPF
DYWGRGTLVTVSS
Polypeptide sequence of the light
chain variable region of anti-PD-L1
antibody
SEQ ID NO: 110
DIVMTQSPDSLAVSLGERATINCKSSQSLFYHSNQ
KHSLAWYQQKPGQPPKLLIYGASTRESGVPDRFSG
SGSGTDFTLTISSLQAEDVAVYYCQQYYGYPYTFG
GGTKVEIK
Polypeptide sequence of the heavy
chain variable region of anti-PD-L1
antibody
SEQ ID NO: 111
QVQLVQSGAEVKKPGASVKVSCKASGYTFTSYWMH
WVRQAPGQGLEWMGRIGPNSGFTSYNEKFKNRVTM
TRDTSTSTVYMELSSLRSEDTAVYYCARGGSSYDY
FDYWGQGTTVTVSS
Polypeptide sequence of the light
chain variable region of anti-PD-L1
antibody
SEQ ID NO: 112
DIVLTQSPASLAVSPGQRATITCRASESVSIHGTH
LMHWYQQKPGQPPKLLIYAASNLESGVPARFSGSG
SGTDFTLTINPVEAEDTANYYCQQSFEDPLTFGQG
TKLEIK
Polypeptide sequence of the heavy
chain of anti-PD-L1 antibody
SEQ ID NO: 113
QVQLQESGPGLVKPSQTLSLTCTVSGGSISNDYWT
WIRQHPGKGLEYIGYISYTGSTYYNPSLKSRVTIS
RDTSKNQFSLKLSSVTAADTAVYYCARSGGWLAPF
DYWGRGTLVTVSSASTKGPSVFPLAPCSRSTSEST
AALGCLVKDYFPEPVTVSWNSGALTSGVHTFPAVL
QSSGLYSLSSVVTVPSSSLGTKTYTCNVDHKPSNT
KVDKRVESKYGPPCPPCPAPEAAGGPSVFLFPPKP
KDTLMISRTPEVTCVVVDVSQEDPEVQFNWYVDGV
EVHNAKTKPREEQFNSTYRVVSVLTVLHQDWLNGK
EYKCKVSNKGLPSSIEKTISKAKGQPREPQVYTLP
PSQEEMTKNQVSLTCLVKGFYPSDIAVEWESNGQP
ENNYKTTPPVLDSDGSFFLYSRLTVDKSRWQEGNV
FSCSVMHEALHNHYTQKSLSLSLGK
Polypeptide sequence of the light
chain of anti-PD-L1 antibody
SEQ ID NO: 114
DIVMTQSPDSLAVSLGERATINCKSSQSLFYHSNQ
KHSLAWYQQKPGQPPKWYGASTRESGVPDRFSGSG
SGTDFTLTISSLQAEDVAVYYCQQYYGYPYTFGGG
TKVEIKRTVAAPSVFIFPPSDEQLKSGTASVVCLL
NNFYPREAKVQWKVDNALQSGNSQESVTEQDSKDS
TYSLSSTLTLSKADYEKHKVYACEVTHQGLSSPVT
KSFNRGEC
Polypeptide sequence of the heavy
chain of anti-PD-L1 antibody
SEQ ID NO: 115
QVQLVQSGAEVKKPGASVKVSCKASGYTFTSYWMH
WVRQAPGQGLEWMGRIGPNSGFTSYNEKFKNRVTM
TRDTSTSTVYMELSSLRSEDTAVYYCARGGSSYDY
FDYWGQGTTVTVSSASTKGPSVFPLAPCSRSTSES
TAALGCLVKDYFPEPVTVSWNSGALTSGVHTFPAV
LQSSGLYSLSSVVTVPSSSLGTKTYTCNVDHKPSN
TKVDKRVESKYGPPCPPCPAPEAAGGPSVFLFPPK
PKDTLMISRTPEVTCVVVDVSQEDPEVQFNWYVDG
VEVHNAKTKPREEQFNSTYRVVSVLTVLHQDWLNG
KEYKCKVSNKGLPSSIEKTISKAKGQPREPQVYTL
PPSQEEMTKNQVSLTCLVKGFYPSDIAVEWESNGQ
PENNYKTTPPVLDSDGSFFLYSRLTVDKSRWQEGN
VFSCSVMHEALHNHYTQKSLSLSLGA
(identical to SEQ ID NO: 11 of
WO 2018/205985)
Polypeptide sequence of the light
chain of anti-PD-Li antibody
SEQ ID NO: 116
DIVLTQSPASLAVSPGQRATITCRASESVSIHGTH
LMHWYQQKPGQPPKLLIYAASNLESGVPARFSGSG
SGTDFTLTINPVEAEDTANYYCQQSFEDPLTFGQG
TKLEIKRTVAAPSVFIFPPSDEQLKSGTASVVCLL
NNFYPREAKVQWKVDNALQSGNSQESVTEQDSKDS
TYSLSSTLTLSKADYEKHKVYACEVTHQGLSSPVT
KSFNRGEC
(identical to SEQ ID NO: 12 of
WO 2018/205985)

Anti-PD-L1/TGFβ Trap molecules useful in the present invention may comprise sequences having at least 85%, at least 90%, at least 95%, at least 96%, at least 97%, at least 98%, or at least 99% sequence identity to any one of SEQ ID NOs: 104-116, as described above.

### III. Production of Antibodies

Methods for producing antibodies, such as those disclosed herein, are known in the art. For example, DNA molecules encoding light chain variable regions and/or heavy chain variable regions can be chemically synthesized using the sequence information provided herein. Synthetic DNA molecules can be ligated to other appropriate nucleotide sequences, including, e.g., constant region coding sequences, and expression control sequences, to produce conventional gene expression constructs encoding the desired antibodies. Production of defined gene constructs is within routine skill in the art.

Nucleic acids encoding desired antibodies can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon, and, optionally, may contain enhancers, and various introns. This expression vector optionally contains sequences encoding all or part of a constant region, enabling an entire, or a part of, a heavy or light chain to be expressed. The gene construct can be introduced into eukaryotic host cells using conventional techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e g., a heavy chain variable region) or a light chain (e g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of a light chain or light chain variable region).

A polypeptide comprising an immunoglobulin heavy chain variable region or light chain variable region can be produced by growing (culturing) a host cell transfected with an expression vector encoding such variable region, under conditions that permit expression of the polypeptide. Following expression, the polypeptide can be harvested and purified or isolated using techniques well known in the art, e.g., affinity tags such as glutathione-S-transferase (GST) and histidine tags.

A monoclonal antibody that binds human TIM-3, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment) can be harvested and purified or isolated using techniques well known in the art, e.g., Protein A, Protein G, affinity tags such as glutathione-S-transferase (GST) and histidine tags. It is within ordinary skill in the art to express the heavy chain and the light chain from a single expression vector or from two separate expression vectors.

### IV. Antibody Modifications

Human monoclonal antibodies can be isolated or selected from phage display libraries including immune, naive and synthetic libraries. Antibody phage display libraries are known in the art, see, e.g., Hoet et al., NATURE BIOTECH. 23:344-348, 2005; Soderlind et al., NATURE BIOTECH. 18:852-856, 2000; Rothe et al., J. MOL. BIOL. 376:1182-1200, 2008; Knappik et al., J. MOL. BIOL. 296:57-86, 2000; and Krebs et al., J. IMMUNOL. METH. 254:67-84, 2001. When used as a therapeutic, human antibodies isolated by phage display may be optimized (e.g., affinity-matured) to improve biochemical characteristics including affinity and/or specificity, improve biophysical properties including aggregation, stability, precipitation and/or non-specific interactions, and/or to reduce immunogenicity Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In some embodiments, isolated human antibodies contain one or more somatic mutations in a framework region. In these cases, framework regions can be modified to a human germline sequence to optimize the antibody (i.e., a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

Antibody Fragments

The proteins and polypeptides of the invention can also include antigen-binding fragments of antibodies. Exemplary antibody fragments include scFv, Fv, Fab, F(ab′)₂, and single domain VHH fragments such as those of camelid origin.

Single-chain antibody fragments, also known as single-chain antibodies (scFvs), are recombinant polypeptides which typically bind antigens or receptors; these fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to assure that the proper three-dimensional folding of the V_{L} and V_{H} domains occurs once they are linked so as to maintain the target molecule binding-specificity of the whole antibody from which the single-chain antibody fragment is derived. Generally, the carboxyl terminus of the V_{L} or V_{H} sequence is covalently linked by such a peptide linker to the amino acid terminus of a complementary V_{L} and V_{H} sequence. Single-chain antibody fragments can be generated by molecular cloning, antibody phage display library or similar techniques. These proteins can be produced either in eukaryotic cells or prokaryotic cells, including bacteria.

Single-chain antibody fragments contain amino acid sequences having at least one of the variable regions or CDRs of the whole antibodies described in this specification, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

Fragments of antibodies that have the same or comparable binding characteristics to those of the whole antibody may also be present. Such fragments may contain one or both Fab fragments or the F(ab′)₂ fragment. The antibody fragments may contain all six CDRs of the whole antibody, although fragments containing fewer than all of such regions, such as three, four or five CDRs, are also functional.

Constant Regions

Unless otherwise specified, constant region antibody amino acid residues are numbered according to the Kabat EU index in Kabat, E.A. et al., (Sequences of proteins of immunological interest. 5th Edition—US Department of Health and Human Services, NIH publication n^{o} 91-3242, pp 662,680,689 (1991)). The antibodies and fragments thereof (e.g., parental and optimized variants) as described herein can be engineered to contain certain constant (i.e., Fc) regions with or lacking a specified effector function (e.g., antibody-dependent cellular cytotoxicity (ADCC)). Human constant regions are known in the art.

The proteins and peptides (e.g., antibodies) of the invention can include a constant region of an immunoglobulin or a fragment, analog, variant, mutant, or derivative of the constant region. In preferred embodiments, the constant region is derived from a human immunoglobulin heavy chain, for example, IgG1, IgG2, IgG3, IgG4, or other classes. In one embodiment, the constant region includes a CH2 domain. In another embodiment, the constant region includes CH2 and CH3 domains or includes hinge-CH2-CH3. Alternatively, the constant region can include all or a portion of the hinge region, the CH2 domain and/or the CH3 domain.

In one embodiment, the constant region contains a mutation that reduces affinity for an Fc receptor or reduces Fc effector function. For example, the constant region can contain a mutation that eliminates the glycosylation site within the constant region of an IgG heavy chain. In some embodiments, the constant region contains mutations, deletions, or insertions at an amino acid position corresponding to Leu234, Leu235, Gly236, Gly237, Asn297, or Pro331 of IgG1 (amino acids are numbered according to Kabat EU index). In a particular embodiment, the constant region contains a mutation at an amino acid position corresponding to Asn297 of IgG1. In alternative embodiments, the constant region contains mutations, deletions, or insertions at an amino acid position corresponding to Leu281, Leu282, Gly283, Gly284, Asn344, or Pro378 of IgG1.

In some embodiments, the constant region contains a CH2 domain derived from a human IgG2 or IgG4 heavy chain Preferably, the CH2 domain contains a mutation that eliminates the glycosylation site within the CH2 domain. In one embodiment, the mutation alters the asparagine within the Gln-Phe-Asn-Ser (SEQ ID NO: 98) amino acid sequence within the CH2 domain of the IgG2 or IgG4 heavy chain. Preferably, the mutation changes the asparagine to a glutamine. Alternatively, the mutation alters both the phenylalanine and the asparagine within the Gln-Phe-Asn-Ser (SEQ ID NO: 98) amino acid sequence. In one embodiment, the Gln-Phe-Asn-Ser (SEQ ID NO: 98) amino acid sequence is replaced with a Gln-Ala-Gln-Ser (SEQ ID NO: 99) amino acid sequence. The asparagine within the Gln-Phe-Asn-Ser (SEQ ID NO: 98) amino acid sequence corresponds to Asn297 of IgG1 (Kabat EU index).

In another embodiment, the constant region includes a CH2 domain and at least a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4, or other suitable classes. More preferably the hinge region is derived from a human IgG1 heavy chain. In one embodiment the cysteine in the Pro-Lys-Ser-Cys-Asp-Lys (SEQ ID NO: 100) amino acid sequence of the IgG1 hinge region is altered. In a preferred embodiment the Pro-Lys-Ser-Cys-Asp-Lys (SEQ ID NO: 100) amino acid sequence is replaced with a Pro-Lys-Ser-Ser-Asp-Lys (SEQ ID NO: 101) amino acid sequence. In one embodiment, the constant region includes a CH2 domain derived from a first antibody isotype and a hinge region derived from a second antibody isotype. In a specific embodiment, the CH2 domain is derived from a human IgG2 or IgG4 heavy chain, while the hinge region is derived from an altered human IgG1 heavy chain.

The alteration of amino acids near the junction of the Fc portion and the non-Fc portion of an antibody or Fc fusion protein can dramatically increase the serum half-life of the Fc fusion protein (PCT publication WO 01/58957, the disclosure of which is hereby incorporated by reference). Accordingly, the junction region of a protein or polypeptide of the present invention can contain alterations that, relative to the naturally-occurring sequences of an immunoglobulin heavy chain, preferably lie within about 10 amino acids of the junction point. These amino acid changes can cause an increase in hydrophobicity. In one embodiment, the constant region is derived from an IgG sequence in which the C-terminal lysine residue is replaced. Preferably, the C-terminal lysine of an IgG sequence is replaced with a non-lysine amino acid, such as alanine or leucine, to further increase serum half-life. In another embodiment, the constant region is derived from an IgG sequence in which the Leu-Ser-Leu-Ser (SEQ ID NO: 102) amino acid sequence near the C-terminus of the constant region is altered to eliminate potential junctional T-cell epitopes. For example, in one embodiment, the Leu-Ser-Leu-Ser (SEQ ID NO: 102) amino acid sequence is replaced with an Ala-Thr-Ala-Thr (SEQ ID NO: 103) amino acid sequence. In other embodiments, the amino acids within the Leu-Ser-Leu-Ser (SEQ ID NO: 102) segment are replaced with other amino acids such as glycine or proline. Detailed methods of generating amino acid substitutions of the Leu-Ser-Leu-Ser (SEQ ID NO: 102) segment near the C-terminus of an IgG1, IgG2, IgG3, IgG4, or other immunoglobulin class molecule have been described in U.S. Patent Publication No. 2003/0166877, the disclosure of which is hereby incorporated by reference.

Suitable hinge regions for the present invention can be derived from IgG1, IgG2, IgG3, IgG4, and other immunoglobulin classes. The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG 1. In some embodiments, the first cysteine within the human IgG1 hinge region is mutated to another amino acid, preferably serine. The IgG2 isotype hinge region has four disulfide bonds that tend to promote oligomerization and possibly incorrect disulfide bonding during secretion in recombinant systems. A suitable hinge region can be derived from an IgG2 hinge; the first two cysteines are each preferably mutated to another amino acid. The hinge region of IgG4 is known to form interchain disulfide bonds inefficiently. However, a suitable hinge region for the present invention can be derived from the IgG4 hinge region, preferably containing a mutation that enhances correct formation of disulfide bonds between heavy chain-derived moieties (Angal S, et al. (1993) Mol. Immunol., 30:105-8).

In accordance with the present invention, the constant region can contain CH2 and/or CH3 domains and a hinge region that are derived from different antibody isotypes, i.e., a hybrid constant region. For example, in one embodiment, the constant region contains CH2 and/or CH3 domains derived from IgG2 or IgG4 and a mutant hinge region derived from IgG1. Alternatively, a mutant hinge region from another IgG subclass is used in a hybrid constant region. For example, a mutant form of the IgG4 hinge that allows efficient disulfide bonding between the two heavy chains can be used. A mutant hinge can also be derived from an IgG2 hinge in which the first two cysteines are each mutated to another amino acid. Assembly of such hybrid constant regions has been described in U.S. Patent Publication No. 2003/0044423, the disclosure of which is hereby incorporated by reference.

In accordance with the present invention, the constant region can contain one or more mutations described herein. The combinations of mutations in the Fc portion can have additive or synergistic effects on the prolonged serum half-life and increased in vivo potency of the molecule. Thus, in one exemplary embodiment, the constant region can contain (i) a region derived from an IgG sequence in which the Leu-Ser-Leu-Ser (SEQ ID NO: 102) amino acid sequence is replaced with an Ala-Thr-Ala-Thr (SEQ ID NO: 103) amino acid sequence; (ii) a C-terminal alanine residue instead of lysine; (iii) a CH2 domain and a hinge region that are derived from different antibody isotypes, for example, an IgG2 CH2 domain and an altered IgG1 hinge region; and (iv) a mutation that eliminates the glycosylation site within the IgG2-derived CH2 domain, for example, a Gln-Ala-Gln-Ser (SEQ ID NO: 99) amino acid sequence instead of the Gln-Phe-Asn-Ser (SEQ ID NO: 98) amino acid sequence within the IgG2-derived CH2 domain.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector agent or joined to the effector agent as a fusion protein. Construction of fusion proteins is within ordinary skill in the art.

### V. Use of Antibodies

The antibodies described herein can be used in a method of downregulating at least one exhaustion marker in a tumor microenvironment, the method comprising exposing the tumor microenvironment to an effective amount of an anti-TIM-3 antibody to downregulate at least one exhaustion marker, such as CTLA-4, LAG-3, PD-1, or TIM-3. Methods for measuring downregulation of exhaustion markers are known in the art, and include, for example, measuring an exhaustion marker on CD4+ and CD8+ T cells following treatment with an anti-TIM-3 antibody.

In certain embodiments, the method can further include exposing the tumor microenvironment to an effective amount of a second therapeutic agent, such as an immune checkpoint inhibitor. Examples of immune checkpoint inhibitors include inhibitors targeting PD-1, PD-L1, or CTLA-4.

The antibodies described herein also can be used in a method of potentiating T cell activation. The method can include exposing the T cell to an effective amount of an anti-TIM-3 antibody, thereby to potentiate the activation of the T cell. In certain embodiments, the method further includes exposing the T cell to an effective amount of a second therapeutic agent, such as an immune checkpoint inhibitor. Methods for measuring T cell activation are described in Example 2.3, and can include measuring IFN-γ production from human PBMCs that were activated by exposure to CEF antigens. In certain embodiments, the method can further include exposing the tumor microenvironment to an effective amount of a second therapeutic agent, such as an anti-PD-L1 antibody.

The antibodies disclosed herein can be used to treat various forms of cancer. In certain embodiments, the cancer or tumor may be selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes. In certain embodiments, the cancer is diffuse large B-cell lymphoma, renal cell carcinoma (RCC), non-small cell lung carcinoma (NSCLC), squamous cell carcinoma of the head and neck (SCCHN), triple negative breast cancer (TNBC) or gastric/stomach adenocarcinoma (STAD). In certain embodiments, the cancer is metastatic or a locally advanced solid tumor. In certain embodiments, no standard therapy exists to treat the cancer and/or the cancer is relapsed and/or refractory from at least one prior treatment. The cancer cells are exposed to a therapeutically effective amount of the antibody so as to inhibit proliferation of the cancer cell. In some embodiments, the antibodies inhibit cancer cell proliferation by at least 40%, 50%, 60%, 70%, 80%, 90%, 95%, 98%, 99%, or 100%.

In some embodiments, the anti-TIM-3 antibody is used in therapy. For example, the antibody can be used to inhibit tumor growth in a mammal (e.g., a human patient). In some embodiments, use of the antibody to inhibit tumor growth in a mammal comprises administering to the mammal a therapeutically effective amount of the antibody. In other embodiments, the anti-TIM-3 antibody can be used for inhibiting proliferation of a tumor cell.

In some embodiments, the anti-TIM-3 antibody is administered in combination with another therapeutic agent, such as radiation (e.g., stereotactic radiation) or an immune checkpoint inhibitor (e.g., targeting PD-1, PD-L1, or CTLA-4). In some embodiments, the anti-TIM-3 antibody is administered in combination with one or more of the following therapeutic agents: anti-PD1/anti-PD-L1 antibodies including Keytruda® (pembrolizumab, Merck & Co.), Opdivo® (nivolumab, Bristol-Myers Squibb), Tecentriq® (atezolizumab, Roche), Imfinzi® (durvalumab, AstraZeneca), TGF-β pathway targeting agents including galunisertib (LY2157299 monohydrate, a small molecule kinase inhibitor of TGF-βRI), LY3200882 (a small molecule kinase inhibitor TGF-βRI disclosed by Pei et al. (2017) CANCER RES 77(13 Suppl):Abstract 955), Metelimumab (an antibody targeting TGF-β1, see Colak et al. (2017) TRENDS CANCER 3(1):56-71), Fresolimumab (GC-1008; an antibody targeting TGF-β1 and TGF-β2), XOMA 089 (an antibody targeting TGF-β1 and TGF-β2; see Mirza et al. (2014) INVESTIGATIVE OPHTHALMOLOGY & VISUAL SCIENCE 55:1121), AVID200 (a TGF-β1 and TGF-β3 trap, see Thwaites et al. (2017) BLOOD 130:2532), Trabedersen/AP12009 (a TGF-β2 antisense oligonucleotide, see Jaschinski et al. (2011) CURR PHARM BIOTECHNOL. 12(12):2203-13), Belagen-pumatucel-L (a tumor cell vaccine targeting TGF-β2, see, e.g., Giaccone et al. (2015) EUR J CANCER 51(16):2321-9); TGB-β pathway targeting agents described in Colak et al. (2017), supra, including Ki26894, SD208, SM16, IMC-TR1, PF-03446962, TEW-7197, and GW788388; any of the immunomodulatory antibodies and fusion proteins described in International Patent Publication No. WO 2011/109789, including those with an immunomodulatory moiety binding to TGF-β, TGF-βR, PD-L1, PD-L2, PD-1, Receptor activator of nuclear factor-κB (RANK) ligand (RANKL), and Receptor activator of nuclear factor-κB (RANK), such as the anti-HER2/neu antibody and TGFβRII ECD fusion protein comprising SEQ ID Nos: 1 and 70 (SEQ II) Nos referenced in the following list are the sequence identifiers as disclosed in International Patent Publication No. WO 2011/109789), the anti-EGFR1 antibody and TGFβRII ECD fusion protein comprising SEQ ID Nos: 2 and 71, the anti-CD20 and TGFβRII ECD fusion protein comprising SEQ ID Nos: 3 and 72, the anti-VEGF antibody and TGFβRII ECD fusion protein comprising SEQ ID Nos: 4 and 73, the anti-CTLA-4 antibody and TGFβRII ECD fusion protein comprising SEQ ID Nos: 5 and 74, the anti-IL-2 Fc and TGFβRII ECD fusion protein comprising SEQ ID Nos: 6 and/or 7, the anti-CD25 antibody and TGFβRII ECD fusion protein comprising SEQ ID Nos: 8 arid 75; the anti-CD25 (Basiliximab) and TGFβRII ECD fusion protein comprising SEQ ID Nos: 9 and 76; the PD-1 ectodomain, Fc and TGFβRII ECD fusion proteins comprising SEQ ID Nos: 11 and/or 12, the TGFβRII ectodomain, Fc and RANK ectodomain fusion proteins comprising SEQ ID Nos: 13 and/or 14, the anti-HER2/neu antibody and PD-1 ectodomain fusion protein comprising SEQ ID Nos: 15 and 70, the anti-EGFR1 antibody and PD-1 ectodomain fusion protein comprising SEQ ID Nos: 16 and 71, the anti-CD20 and PD-1 ectodomain fusion protein comprising SEQ ID Nos: 17 and 72, the anti-VEGF antibody and PD-1 ectodomain fusion protein comprising SEQ ID Nos: 18 and 73, the anti-CTLA-4 antibody and PD-1 ectodomain fusion protein comprising SEQ ID Nos: 19 and 74, the anti-CD25 antibody and PD-1 ectodomain fusion protein comprising SEQ ID Nos: 20 and 75; the anti-CD25 (Basiliximab) and PD-1 ectodomain fusion protein comprising SEQ ID Nos: 21 and 76; the IL-2, Fc and PD-1 ectodomain fusion proteins comprising SEQ ID NO: 16 and/or 23, the anti-CD4 antibody and PD-1 extracellular domain fusion protein comprising SEQ ID Nos: 24 and 77, the PD-1 ectodomain, Fc, RANK ECD fusion proteins comprising SEQ ID NO: 16 and/or 23, the anti-HER2/neu antibody and RANK ECD fusion protein comprising SEQ ID Nos: 27 and 70, the anti-EGFR1 antibody and RANK ECD fusion protein comprising SEQ ID Nos: 28 and 71, the anti-CD20 and RANK ECD fusion protein comprising SEQ ID Nos: 29 and 72, the anti-VEGF antibody and RANK ECD fusion protein comprising SEQ ID Nos: 30 and 73, the anti-CTLA-4 antibody and RANK ECD fusion protein comprising SEQ ID Nos: 31 and 74, the anti-CD25 antibody and RANK ECD fusion protein comprising SEQ ID Nos: 32 and 75; the anti-CD25 (Basiliximab) and RANK ECD fusion protein comprising SEQ ID Nos: 33 and 76, the IL-2, Fc and RANK ECD fusion proteins comprising SEQ ID NOs: 34 and/or 35, the anti-CD4 antibody and RANK ECD fusion protein comprising SEQ ID Nos: 36 and 77, the anti-TNFα antibody and PD-1 ligand 1 or PD-1 ligand 2 fusion proteins comprising SEQ ID Nos: 37 and 78, the TNFR2 extracellular biding domain, Fc and PD-1 ligand fusion protein comprising SEQ ID NO: 38 and/or 39, the anti-CD20 and PD-L1 fusion protein comprising SEQ ID Nos: 40 and 72, the anti-CD25 antibody and PD-L1 fusion protein comprising SEQ ID Nos: 41 and 75, the anti-CD25 (Basiliximab) and PD-1 ectodomain fusion protein comprising SEQ ID Nos: 42 and 76, the IL-2, Fc and PD-L1 fusion proteins comprising SEQ ID Nos: 43 and/or 44, the anti-CD4 antibody and PD-L1 fusion protein comprising SEQ ID Nos: 45 and 77, the CTLA-4 ECD, Fc (IgG Cγ1) and PD-L1 fusion proteins comprising SEQ ID Nos: 46 and/or 47, the Fc (IgG Cγ1) and PD-L1 fusion proteins comprising SEQ ID Nos: 48 and/or 49, the anti-TNF-α antibody and TGF-β fusion protein comprising SEQ ID Nos: 50 and 77, the TNFR2 extracellular binding domain , Fc and TGF-β fusion proteins comprising SEQ 1D Nos: 51 and/or 52, the anti-CD20 and TGF-β fusion protein comprising SEQ ID Nos: 53 and 72, the anti-CD25 antibody and TGF-β fusion protein comprising SEQ ID Nos: 54 and 75, the anti-CD25 (Basiliximab) and TGF-β fusion protein comprising SEQ ID Nos: 55 and 76, the IL-2, Fc and TGF-β fusion proteins comprising SEQ ID Nos: 56 and/or 57, the CTLA-4 ECD, Fc (IgG Cγ1) and TGF-β fusion proteins comprising SEQ ID Nos: 59 and/or 60, the anti-TNF-α antibody and RANK fusion protein comprising SEQ ID Nos: 61 and 78, the TNFR2 extracellular binding domain , Fc and RANK fusion proteins comprising SEQ ID Nos: 62 and/or 63, the CTLA-4 ECD, Fc (IgG Cγ1) and RANK fusion proteins comprising SEQ ID Nos: 64 and/or 65, the RANK, Fc, and TGF-β fusion proteins comprising SEQ ID Nos: 66 and/or 67, and the RANK, Fc, and PD-L1 fusion proteins comprising SEQ ID Nos: 68 and/or 69.

Methods of Treatment

As used herein, “treat,” “treating,” and “treatment” mean the treatment of a disease in a mammal, e.g., in a human. This includes: (a) inhibiting the disease, i.e., arresting its development; and (b) relieving the disease, i.e., causing regression of the disease state.

Generally, a therapeutically effective amount of anti-TIM-3 antibody or another therapeutic agent described herein (alone or in combination with another treatment, e.g., a second therapeutic agent) is in the range of about 0.1 mg/kg to about 100 mg/kg, e.g., about 1 mg/kg to about 100 mg/kg, e.g., 1 mg/kg to 10 mg/kg. In certain embodiments, a therapeutically effective amount of an anti-TIM-3 antibody or another therapeutic agent described herein can be administered at a dose from about 0.1 to about 1 mg/kg, from about 0.1 to about 5 mg/kg, from about 0.1 to about 10 mg/kg, from about 0.1 to about 25 mg/kg, from about 0.1 to about 50 mg/kg, from about 0.1 to about 75 mg/kg, from about 0.1 to about 100 mg/kg, from about 0.5 to about 1 mg/kg, from about 0.5 to about 5 mg/kg, from about 0.5 to about 10 mg/kg, from about 0.5 to about 25 mg/kg, from about 0.5 to about 50 mg/kg, from about 0.5 to about 75 mg/kg, from about 0.5 to about 100 mg/kg, from about 1 to about 5 mg/kg, from about 1 to about 10 mg/kg, from about 1 to about 25 mg/kg, from about 1 to about 50 mg/kg, from about 1 to about 75 mg/kg, from about 1 to about 100 mg/kg, from about 5 to about 10 mg/kg, from about 5 to about 25 mg/kg, from about 5 to about 50 mg/kg, from about 5 to about 75 mg/kg, from about 5 to about 100 mg/kg, from about 10 to about 25 mg/kg, from about 10 to about 50 mg/kg, from about 10 to about 75 mg/kg, from about 10 to about 100 mg/kg, from about 25 to about 50 mg/kg, from about 25 to about 75 mg/kg, from about 25 to about 100 mg/kg, from about 50 to about 75 mg/kg, from about 50 to about 100 mg/kg, from about 75 to about 100 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study designed to run from 0.5 mg/kg to 30 mg/kg.

In certain embodiments, the anti-TIM-3 antibody or another therapeutic agent described herein (alone or in combination with another treatment, e.g., a second therapeutic agent) can be administered as a flat (fixed) dose (rather than in proportion to a mammal's body weight, i.e., a mg/kg dosage). A therapeutically effective amount of an anti-TIM-3 antibody can be a flat (fixed) dose of about 5 mg to about 3500 mg. For example, the dose can be from about 5 to about 250 mg, from about 5 to about 500 mg, from about 5 to about 750 mg, from about 5 to about 1000 mg, from about 5 to about 1250 mg, from about 5 to about 1500 mg, from about 5 to about 1750 mg, from about 5 to about 2000 mg, from about 5 to about 2250 mg, from about 5 to about 2500 mg, from about 5 to about 2750 mg, from about 5 to about 3000 mg, from about 5 to about 3250 mg, from about 5 to about 3500 mg, from about 250 to about 500 mg, from about 250 to about 750 mg, from about 250 to about 1000 mg, from about 250 to about 1250 mg, from about 250 to about 1500 mg, from about 250 to about 1750 mg, from about 250 to about 2000 mg, from about 250 to about 2250 mg, from about 250 to about 2500 mg, from about 250 to about 2750 mg, from about 250 to about 3000 mg, from about 250 to about 3250 mg, from about 250 to about 3500 mg, from about 500 to about 750 mg, from about 500 to about 1000 mg, from about 500 to about 1250 mg, from about 500 to about 1500 mg, from about 500 to about 1750 mg, from about 500 to about 2000 mg, from about 500 to about 2250 mg, from about 500 to about 2500 mg, from about 500 to about 2750 mg, from about 500 to about 3000 mg, from about 500 to about 3250 mg, from about 500 to about 3500 mg, from about 750 to about 1000 mg, from about 750 to about 1250 mg, from about 750 to about 1500 mg, from about 750 to about 1750 mg, from about 750 to about 2000 mg, from about 750 to about 2250 mg, from about 750 to about 2500 mg, from about 750 to about 2750 mg, from about 750 to about 3000 mg, from about 750 to about 3250 mg, from about 750 to about 3500 mg, from about 1000 to about 1250 mg, from about 1000 to about 1500 mg, from about 1000 to about 1750 mg, from about 1000 to about 2000 mg, from about 1000 to about 2250 mg, from about 1000 to about 2500 mg, from about 1000 to about 2750 mg, from about 1000 to about 3000 mg, from about 1000 to about 3250 mg, from about 1000 to about 3500 mg, from about 1250 to about 1500 mg, from about 1250 to about 1750 mg, from about 1250 to about 2000 mg, from about 1250 to about 2250 mg, from about 1250 to about 2500 mg, from about 1250 to about 2750 mg, from about 1250 to about 3000 mg, from about 1250 to about 3250 mg, from about 1250 to about 3500 mg, from about 1500 to about 1750 mg, from about 1500 to about 2000 mg, from about 1500 to about 2250 mg, from about 1500 to about 2500 mg, from about 1500 to about 2750 mg, from about 1500 to about 3000 mg, from about 1500 to about 3250 mg, from about 1500 to about 3500 mg, from about 1750 to about 2000 mg, from about 1750 to about 2250 mg, from about 1750 to about 2500 mg, from about 1750 to about 2750 mg, from about 1750 to about 3000 mg, from about 1750 to about 3250 mg, from about 1750 to about 3500 mg, from about 2000 to about 2250 mg, from about 2000 to about 2500 mg, from about 2000 to about 2750 mg, from about 2000 to about 3000 mg, from about 2000 to about 3250 mg, from about 2000 to about 3500 mg, from about 2250 to about 2500 mg, from about 2250 to about 2750 mg, from about 2250 to about 3000 mg, from about 2250 to about 3250 mg, from about 2250 to about 3500 mg, from about 2500 to about 2750 mg, from about 2500 to about 3000 mg, from about 2500 to about 3250 mg, from about 2500 to about 3500 mg, from about 2750 to about 3000 mg, from about 2750 to about 3250 mg, from about 2750 to about 3500 mg, from about 3000 to about 3250 mg, from about 3000 to about 3500 mg, or from about 3250 to about 3500 mg. Human dosage can be optimized, e.g., in a conventional Phase I dose escalation study designed to run from a flat (fixed) dose of 5 mg to 3200 mg.

In a preferred embodiment, the anti-TIM-3 antibody is administered in a flat (fixed) dose of from about 20 mg to about 1600 mg. For example, the dose can be from about 20 mg to about 80 mg, from about 20 mg to about 240 mg, from about 20 mg to about 800 mg, from about 20 mg to about 1600 mg, from about 80 mg to about 240 mg, from about 80 mg to about 800 mg, from about 80 mg to about 1600 mg, from about 240 mg to about 800 mg, from about 240 mg to about 1600 mg, from about 800 mg to about 1600 mg. In certain embodiments the anti-TIM-3 antibody is administered in a flat (fixed) dose of about 20 mg, about 80 mg, about 240 mg, about 800 mg or about 1600 mg.

In certain embodiments, the anti-TIM-3 antibody is administered in combination with an anti-PD-L1/TGFβ Trap fusion protein (e.g., bintrafusp alfa), wherein the anti-PD-L1/TGFβ Trap fusion protein is administered at a flat (fixed) dose of about 800 mg to about 2600 mg (e.g., about 800 mg to about 1100 mg, about 800 mg to about 1200 mg, about 800 mg to about 1500 mg, about 800 mg to about 2000 mg, about 800 mg to about 2300 mg, about 800 mg to about 2400 mg, about 800 mg to about 2600 mg, about 1100 mg to about 1200 mg, about 1100 mg to about 1500 mg, about 1100 mg to about 2000 mg, about 1100 mg to about 2300 mg, about 1100 mg to about 2400 mg, about 1100 mg to about 2600 mg, about 1200 mg to about 1500 mg, about 1200 mg to about 2000 mg, about 1200 mg to about 2300 mg, about 1200 mg to about 2400 mg, about 1200 mg to about 2600 mg, about 1500 mg to about 2000 mg, about 1500 mg to about 2300 mg, about 1500 mg to about 2400 mg, about 1500 mg to about 2600 mg, about 2000 mg to about 2300 mg, about 2000 mg to about 2400 mg, about 2000 mg to about 2600 mg, about 2300 mg to about 2400 mg, about 2300 mg to about 2600 mg, or about 2400 mg to about 2600 mg. In certain embodiments, the anti-PD-L1/TGFβ Trap fusion protein is administered at a flat (fixed) dose of about 1200 mg. In certain further embodiments, the anti-TIM-3 antibody is administered in combination with an anti-PD-L1/TGFβ Trap fusion protein (e.g., bintrafusp alfa), wherein the anti-PD-L1/TGFβ Trap fusion protein is administered at a flat (fixed) dose of about 2400 mg.

Dosing frequency can vary, depending on factors such as route of administration, dosage amount, serum half-life of the antibody, and the disease being treated. Exemplary dosing frequencies are once per week, once every two weeks, once every three weeks and once every four weeks. In some embodiments, dosing is once every two weeks. In certain embodiments, the anti-TIM-3 antibody is administered in combination with an anti-PD-L1/TGFβ Trap fusion protein (e.g., bintrafusp alfa) every two weeks, wherein anti-PD-L1/TGFβ Trap fusion protein is administered at a flat (fixed) dose of about 1200 mg. In certain embodiments, the anti-TIM-3 antibody is administered in combination with an anti-PD-L1/TGFβ Trap fusion protein (e.g., bintrafusp alfa) every three weeks, wherein anti-PD-LUTGFβ Trap fusion protein is administered at a flat (fixed) dose of about 2400 mg.

A preferred route of administration is parenteral, e.g., intravenous infusion. Formulation of monoclonal antibody-based drugs is within ordinary skill in the art. In some embodiments, the antibody is lyophilized, and then reconstituted in buffered saline, at the time of administration.

For therapeutic use, an antibody preferably is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing antibodies, such as those disclosed herein, can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for monoclonal antibodies is IV infusion. Useful formulations can be prepared by methods well known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

The intravenous drug delivery formulation of the present disclosure for use in a method of treating cancer or inhibiting tumor growth in a mammal may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may be freeze-dried (lyophilized) and contained. In certain embodiments, the about 40 mg-about 100 mg of freeze-dried formulation may be contained in one vial. In certain embodiments, the formulation may be a liquid formulation of a protein product that includes an anti-TIM-3 antibody as described herein and stored as about 250 mg/vial to about 2000 mg/vial.

Liquid Formulation

This disclosure provides a liquid aqueous pharmaceutical formulation including a therapeutically effective amount of the protein of the present disclosure (e.g., anti-TIM-3 antibody) in a buffered solution forming a formulation for use in a method of treating cancer or inhibiting tumor growth in a mammal.

These compositions for use in a method of treating cancer or inhibiting tumor growth in a mammal may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides for use in a method of treating cancer or inhibiting tumor growth in a mammal, a formulation with an extended shelf life including a protein of the present disclosure (e.g., an anti-TIM-3 antibody), in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation for use in a method of treating cancer or inhibiting tumor growth in a mammal is prepared including a protein of the present disclosure (e.g., an anti-TIM-3 antibody) in a pH-buffered solution. The buffer of this invention may have a pH ranging from about 4 to about 8, e.g., from about 4 to about 8, from about 4.5 to about 8, from about 5 to about 8, from about 5.5 to about 8, from about 6 to about 8, from about 6.5 to about 8, from about 7 to about 8, from about 7.5 to about 8, from about 4 to about 7.5, from about 4.5 to about 7.5, from about 5 to about 7.5, from about 5.5 to about 7.5, from about 6 to about 7.5, from about 6.5 to about 7.5, from about 4 to about 7, from about 4.5 to about 7, from about 5 to about 7, from about 5.5 to about 7, from about 6 to about 7, from about 4 to about 6.5, from about 4.5 to about 6.5, from about 5 to about 6.5, from about 5.5 to about 6.5, from about 4 to about 6.0, from about 4.5 to about 6.0, from about 5 to about 6, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In certain embodiments, the formulation for use in a method of treating cancer or inhibiting tumor growth in a mammal includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86 mg/mL), and about 6.2 mg/mL of sodium chloride (e.g., 6.165 mg/mL). In certain embodiments, the buffer system includes about 1-1.5 mg/mL of citric acid, about 0.25 to about 0.5 mg/mL of sodium citrate, about 1.25 to about 1.75 mg/mL of disodium phosphate dihydrate, about 0.7 to about 1.1 mg/mL of sodium dihydrogen phosphate dihydrate, and 6.0 to 6.4 mg/mL of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also alter with respect to the molecular weight of the polyol. For example, a lower amount of a monosaccharide (e.g. mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to about 15 mg/mL. In certain embodiments, the concentration of mannitol may be about 10-about 14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g. polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hilfsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 u mass decrease of the parent peptide. The subsequent hydrolysis results in an 18 u mass increase. Isolation of the succinimide intermediate is difficult due to instability under aqueous conditions. As such, deamidation is typically detectable as 1 u mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation for use in a method of treating cancer or inhibiting tumor growth in a mammal of the present disclosure may be preserved under conditions of pH and humidity to prevent deamidation of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In one embodiment, the liquid formulation contains 10 mg/mL M6903, 8% (w/v) Trehalose, 10 mM L-Histidine and 0.05% Polysorbate 20, pH 5.5. Prior to administration of M6903 by intravenous infusion, the solution is diluted in sterile 0.9% sodium chloride.

Lyophilized Formulation

The lyophilized formulation for use in a method of treating cancer or inhibiting tumor growth in a mammal of the present disclosure includes the anti-TIM-3 antibody molecule and a lyoprotectant. The lyoprotectant may be sugar, e.g., disaccharides. In certain embodiments, the lycoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between about 6 to about 8. In certain embodiments, the pH range for the lyophilized drug product may be from about 7 to about 8.

In certain embodiments, a salt or buffer components may be added in an amount of about 10 mM-about 200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e. g. , facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product for use in a method of treating cancer or inhibiting tumor growth in a mammal may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g. phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

Practice of the invention will be more fully understood from the foregoing examples, which are presented herein for illustrative purposes only, and should not be construed as limiting the invention in any way.

### EXAMPLES

### Example 1

### Epitope Manning

1.1 Co-Crystallization of TIM-3 with 3903E11 (VL1.3, VH1.2) Fab

A crystal structure of the complex of TIM-3 ECD and the Fab fragment of the 3903E11 (VL1.3,VH1.2) (heavy chain: SEQ ID NO: 47; light chain: SEQ ID NO: 48) was determined. Human TIM-3 (SEQ ID NO: 49 (amino acid); SEQ ID NO: 50 (nucleotide)) was expressed in E. coli inclusion bodies, refolded, and purified by affinity and size exclusion chromatography. The Fab fragment of 3903E11 (VL1.3,VH1.2) was expressed as a His-tagged construct in Expi293F cells and purified by affinity chromatography. The complex of TIM-3 and 3903E11 (VL1.3,VH1.2) Fab fragment was formed and purified by gel filtration chromatography yielding a homogenous protein with a purity greater than 95%.

Crystals of Fab 3903E11 (VL1.3,VH1.2) in complex with human TIM-3 were grown by mixing 0.75 μl protein solution (21.8 mg/mL in 20 mM TrisHCL pH 8.0, 100 mM NaCl) with 0.5 μl reservoirs solution (20% PEG400 (v/v), 0.1 M Tris HCl pH 8.0) at 4° C. using hanging drop vapor diffusion method.

Crystals were flash-frozen and measured at a temperature of 100 K. The X-ray diffraction data was collected at the SWISS LIGHT SOURCE (SLS, Villigen, Switzerland) using cryogenic conditions. The crystals belong to space group C 2 2 21. Data were processed using the programs XDS and XSCALE.

The phase in-formation necessary to determine and analyse the structure was obtained by molecular replacement. The published structures PDB-ID 5F71 and 1NL0 were used as search models for TIM3 and the Fab fragment, respectively. Subsequent model building and refinement was performed according to standard protocols with the software packages CCP4 and COOT. For the calculation of the free R-factor, a measure to cross-validate the correctness of the final model, about 0.9% of measured reflections were excluded from the refinement procedure (see TABLE 1). TLS refinement (using REFMAC5, CCP4) was carried out, which resulted in lower R-factors and higher quality of the electron density map. The ligand parameterisation and generation of the corresponding library files were carried out with CHEMSKETCH and LIBCHECK (CCP4), respectively. The water model was built with the “Find waters”-algorithm of COOT by putting water molecules in peaks of the Fo-Fc map contoured at 3.0 followed by refinement with REFMAC5 and checking all waters with the validation tool of COOT. The criteria for the list of suspicious waters were: B-factor greater 80 Å2, 2Fo-Fc map less than 1.2 Å, distance to closest contact less than 2.3 Å or more than 3.5 Å. The suspicious water molecules and those in the ligand binding site (distance to ligand less than The suspicious water molecules and those in the ligand binding site (distance to ligand less than 10 Å) were checked manually. The Ramachandran Plot of the final model shows 85.4% of all residues in the most favored region, 13.9% in the additionally al-lowed region, and 0.2% in the generously allowed region. The residues Arg81(A), Arg81(B), Va153(L), Asp153(L), Va153(M), Asp153(M), Va153(N), Va153(O), and Asp153(O) are found in the disallowed region of the Ramachandran plot. They are either confirmed by the electron density map or could not be modelled in another sensible conformation.

TABLE 1
Data collection and processing statistics for TIM3

| X-ray Source | PXI/X06A (SLS¹) |
|---|---|
| Wavelength [Å] | 1.0000 |
| Detector | EIGER × 16M |
| Temperature [K] | 100 |
| Space Group | C 2 2 2₁ |
| Cell: a; b; c; [Å] | 119.35; 270.12; 197.89 |
| α; β; γ; [°] | 90.0; 90.0; 9.0 |
| Resolution [Å] | 3.06 (3.31-3.06) |
| Unique reflections | 59975 (12146) |
| Multiplicity | 3.8 (3.9) |
| Completeness [%] | 99.0 (96.7) |
| Rsym [%]³ | 7.5 (50.2) |
| Rmeas [%]⁴ | 8.7 (58.3) |
| Mean (I)/sd⁵ | 15.36 (2.95) |

¹SWISS LIGHT SOURCE (SLS, Villigen, Switzerland)
²values in parenthesis refer to the highest resolution
${ }^{3} Rsym = \frac{}{} \text{with} = \frac{}{} \sum \text{where} \text{is the intensity value}$ of the ith measurement of h
${ }^{4} Rmeas = \frac{}{} \text{with} = \frac{1}{} where \text{is the intensity value of}$ the ith measurement of h
⁵calculated with independent reflections
$\text{?} \text{indicates text missing or illegible when filed}$

Epitope residues are defined as all residues of TIM-3 with a heavy atom within 5 angstroms of a heavy atom of 3903E11 (VL1.3,VH1.2 Fab. Distances were measured from the final crystallographic coordinates using the BioPython package. Only contacts present in 3 of the 4 complexes of the asymmetric unit are reported (TABLE 2). TABLE 2 tabulates interactions between TIM-3 and 3903E11 (VL1.3,VH1.2). TIM-3 residues are numbered as in Uniprot Code Q8TDQ0-1 (SEQ ID NO: 51). The antibody residues are numbered with reference to SEQ ID NO:47 (heavy chain, “H”) and SEQ ID NO:48 (light chain, “L”). Residues listed here have at least one heavy atom within 5 angstroms of a heavy atom across the interface.

TABLE 2
Interactions between huTIM-3 and mAb 3903E11
(VL1.3, VH1.2)

| huTIM-3 | | 3903E11 (VL1.3, VH1.2) | | |
|---|---|---|---|---|
| Amino | | Amino | | |
| Acid | Number | Acid | Number | Chain |
| PRO | 50 | SER | 54 | H |
| LYS | 55 | TYR | 34 | L |
| GLY | 56 | TYR | 34 | L |
| ALA | 57 | TYR | 32 | L |
| | | TYR | 34 | L |
| CYS | 58 | TYR | 34 | L |
| | | ALA | 94 | L |
| PRO | 59 | TRP | 101 | H |
| | | TYR | 34 | L |
| | | TYR | 93 | L |
| | | ALA | 94 | L |
| VAL | 60 | TRP | 47 | H |
| | | TYR | 59 | H |
| | | TRP | 101 | H |
| | | GLY | 102 | H |
| | | TYR | 93 | L |
| | | ALA | 94 | L |
| | | ASP | 95 | L |
| | | SER | 96 | L |
| | | VAL | 97 | L |
| PHE | 61 | ALA | 33 | H |
| | | SER | 35 | H |
| | | TRP | 47 | H |
| | | ALA | 50 | H |
| | | TYR | 59 | H |
| | | ALA | 99 | H |
| | | ASN | 100 | H |
| | | TRP | 101 | H |
| | | GLY | 102 | H |
| | | PHE | 104 | H |
| | | VAL | 97 | L |
| GLU | 62 | ALA | 33 | H |
| | | SER | 52 | H |
| | | VAL | 53 | H |
| | | TYR | 59 | H |
| | | ASN | 100 | H |
| | | TRP | 101 | H |
| CYS | 63 | TRP | 101 | H |
| GLY | 64 | TRP | 101 | H |
| | | TYR | 34 | L |
| ASN | 65 | TRP | 101 | H |
| | | ASP | 52 | L |
| | | LYS | 55 | L |
| VAL | 66 | TRP | 101 | H |
| ARG | 69 | SER | 31 | H |
| | | VAL | 53 | H |
| GLU | 72 | SER | 54 | H |
| ARG | 111 | TYR | 59 | H |
| GLN | 113 | SER | 52 | H |
| | | VAL | 53 | H |
| | | SER | 54 | H |
| | | SER | 57 | H |
| | | TYR | 59 | H |
| ILE | 114 | GLY | 56 | H |
| | | SER | 57 | H |
| PRO | 115 | GLY | 56 | H |
| | | SER | 57 | I |
| GLY | 116 | GLY | 56 | H |
| | | SER | 57 | H |
| | | THR | 58 | H |
| ILE | 117 | THR | 58 | H |
| | | TYR | 60 | H |
| | | LYS | 65 | H |
| MET | 118 | SER | 57 | H |
| | | THR | 58 | H |
| | | TYR | 59 | H |
| | | TYR | 60 | H |
| | | LYS | 65 | H |
| ASN | 119 | SER | 57 | H |
| ASP | 120 | SER | 57 | H |
| | | TYR | 59 | H |
| LYS | 122 | ASP | 95 | L |

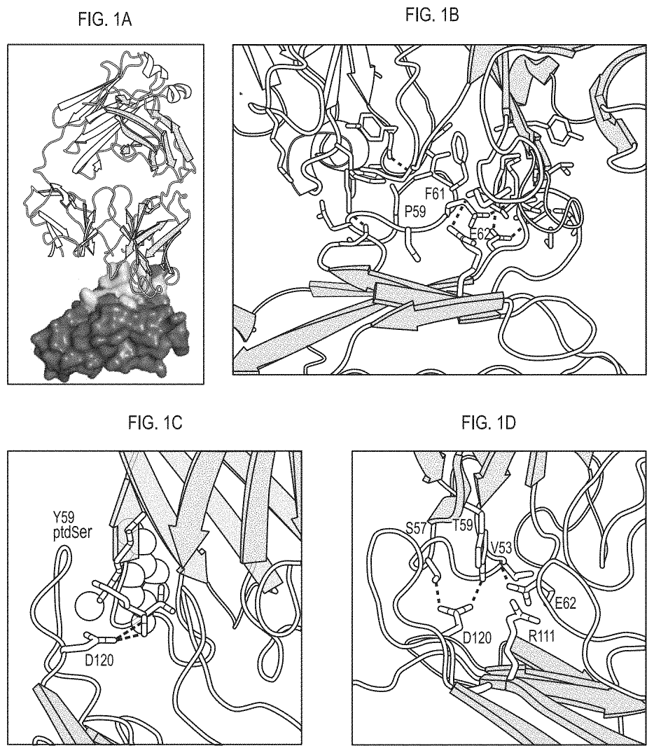

The crystal structure of human TIM-3 in complex with M6903 is shown in FIG. 1A-D. FIG. 1A shows an overview of the Fab portion of M6903 (upper structure) bound to TIM-3 shown as a surface representation. Extensive contacts made on TIM-3 (bottom structure) are shown as the lighter portion of TIM-3. The majority of the contact occurs with the heavy chain and the third complementarity determining region of the light chain (CDR-L3) of M6903. FIG. 1B shows the epitope hotspot residues of TIM-3 (e.g., P59 and F61 and E62). The residues form extensive hydrophobic and electrostatic interactions to M6903. FIG. 1C shows the polar head group of ptdSer (light-colored sticks) and the coordinating calcium ion (sphere) have been modeled into the structure of M6903-bound TIM-3 by superposition with the structure of murine TIM-3 (DeKruyff et al. (2010), supra). The binding site of ptdSer coincides with the placement of Y59 (group of spheres) of the heavy chain from M6903. Hydrogen bonds from D120 on TIM-3 to ptdSer or M6903, respectively, are shown as dotted lines. FIG. 1D shows the polar interactions of M6903 with the CEACAM-1 binding residues of TIM-3 are shown with dashed lines.

1.2 Mutagenesis

To identify residues of the epitope which contribute energetically to binding selected residues in human TIM-3 were mutated either to alanine (large to small) or to glycine if the selected residue was alanine or to switch the charge of the side-chain In total 11 human TIM-3 point mutants were designed, expressed and purified in HEK cells, and tested for binding to 3903E11 (VL1.3,VH1.2)-IgG2h(FN-AQ,322A)-delK antibody (M6903) using surface plasmon resonance using a GE Healthcare Biacore 4000 instrument as follows. Goat anti-human Fc antibody (Jackson Immunoresearch Laboratories #109-005-098) was first immobilized on BIAcore carboxymethylated dextran CM5 chip using direct coupling to free amino groups following the procedure described by the manufacturer. Antibodies were then captured on the CM5 biosensor chip to achieve approximately 200 response units (RU). Binding measurements were performed using the running HBS-EP+ buffer. A 2-fold dilution series starting at 100 nM of the anti-TIM-3 antibodies were injected at a flow rate of 30 μl/min at 25° C. Association rates (kon, M-1s-1) and dissociation rates (koff, s-1) were calculated using a simple 1:1 Langmuir binding model (Biacore 4000 Evaluation Software). The equilibrium dissociation constant (KD, M) was calculated as the ratio of koff/kon. The affinity of the antibody for wild-type and each mutant was determined. Results are summarized in TABLE 3. Mutants were compared to wild-type TIM-3 (hu TIM-3). The temperature midpoint of fluorescently monitored thermal denaturation is given for the wild-type and mutant proteins. The percent monomer as determine by analytical SEC is given. For KD and T1/2, the mean and standard deviation is given where n>1. It was important to confirm that the lack of binding for a particular point mutant was indeed due to loss of residue interaction and not to global unfolding of the antigen. The structural integrity of the mutated proteins was confirmed using a fluorescence monitored thermal unfolding (FMTU) assay in which the protein is incubated with a dye that is quenched in aqueous solution but fluoresces when bound by exposed hydrophobic residues. As the temperature increases, thermal denaturation of the protein exposes the hydrophobic core residues and this can be monitored by an increase in fluorescence of the dye. A melting curve is fit to the data with the Boltzmann equation outlined in Equation 1, adapted from (Bullock et al. 1997) to determine the temperature at the inflection point of the curve (T1/2). The calculated T1/2 are reported in TABLE 3.

$$F = {F}_{min} + \frac{{F}_{max} - {F}_{min}}{1 + {e}^{\frac{{T}_{m} - x}{dx}}} \qquad Equation 1$$

TABLE 3
Summary of TIM-3 Variant Binding to Antibodies

| | Binding Affinity KD (nM) | | | | ΔΔGmut (kcal/mol) | | | | Stability | |
|---|---|---|---|---|---|---|---|---|---|---|
| Ligand | M6903 | 27.12 E12 | h03 | mab15 | M6903 | 2712 E12 | h03 | mab15 | % Monomer | T_{1/2} (° C.) |
| TIM3 | 6.2 ± 1.5 | 51.6 ± 10.8 | 0.3 ± 0.5 | 0.7 ± 0.2 | NA | NA | NA | NA | 94 | 52 |
| P59A | NB | 12.3 ± 1.9 | 0.9 ± 0.03 | 0.7 ± 0.2 | >1.6 | −0.2 | 0.6 | −0.03 | 83 | 48 |
| V60A | 3.7 ± 0.1 | 23.4 ± 1.0 | 0.4 ± 0.04 | 0.7 ± 0.4 | −0.3 | −0.2 | 0.2 | −0.05 | 94 | 51 |
| F61A | NB | 28.6 ± 1.2 | 0.6 ± 0.09 | 0.9 ± 0.3 | >1.6 | −0.1 | 0.3 | −0.1 | 100 | nd |
| E62A | 106.1 ± 32 | 28.3 ± 0.2 | 0.4 ± 0.05 | 0.5 ± 0.1 | >1.6 | −0.3 | 0.1 | −0.3 | 97 | 51 |
| R111A | | | | | | | | | 23 | nd |
| R111E | | | | | | | | | 83 | nd |
| I114A | 29.3 ± 0.7 | 26.7 ± 2.6 | 0.7 ± 0.01 | 0.6 ± 0.1 | 0.9* | −0.1 | 0.5 | −0.1 | 95 | nd |
| M118A | 9.7 ± 0.6 | 49.7 ± 4.5 | 0.7 ± 0.09 | 1.1 ± 0.4 | 0.3 | −0.04 | 0.5 | 0.2 | 99 | nd |
| N119A | 17.2 ± 0.7 | 29.1 ± 0.3 | 0.7 ± 0.08 | 1.2 ± 0.4 | 0.6 | −0.3 | 0.4 | 0.3 | 79 | nd |
| K122A | 46.6 ± 0.2 | 22.1 ± 3.3 | 8.4 ± 0.44 | 1.5 ± 0.6 | 1.2 | −0.5 | 1.9 | 0.4 | 90 | 47 |
| F123A | | | | | | | | | 79 | nd |

NB = No Detectable Binding;
nd = not determined for data quality control;
*= potential conformational destabilization or indirect contacts

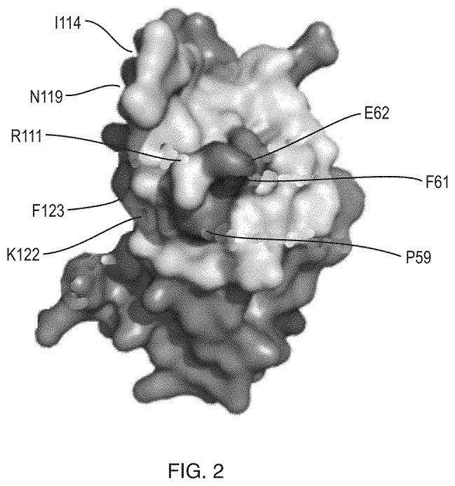

M6903 showed a decrease or loss of binding for the TIM-3 single point mutants P59A, F61A, E62A, I114A, N119A, and K122A (see TABLE 3). Residues P59A, F61A, E62A, I114A, N119A, and K122A reside on the face of one beta sheet of the immunoglobulin fold as shown with the model (see FIG. 2) and are present in the CC′ and FG loops of human TIM-3, loops which have been shown to be involved in Ptd-Ser binding. Contact with the sidechain of Ile-114 by M6903 is not evident; the moderate deleterious effects due to mutation are explained as local destabilization of the loop region. The closest cross-interface contacts for Lys-122 are 4.7 Å and occur with backbone carbonyls of the antibody. Water-bridging interactions are possible at this distance but could not be observed given the resolution of the crystal structure. The deleterious effects of the K122A mutation may be explained if the gap is bridged via water bridging.

TIM-3 mutants R111 and F123 showed low stability as assessed by SEC, FMTU, and any reduced binding observed for R111 and F123 mutants likely due to destabilization of the protein and not critical interactions with the antibody. Therefore, TABLE 3 indicates hotspot residues for binding of M6903 to include P59A, F61A, and E62A (see also FIG. 2).

The experiment was repeated using known antibodies ABTIM3-h03 ABTIM3-mAB 15, and 27.12E12. Results are shown in TABLE 3 and TABLE 4. For known antibody mAb h03, residues P59A, I114A, M118A, and K122A are identified as residues in the binding interface given the effect on binding. In particular, K122 and F123 are shown as hotspots for mAb h03. These positions are in the reported binding footprint for mAb h03 (US20150218274A1, hum21 is Fab form of h03). Accordingly, while some mutant hu TIM-3 proteins resulted in loss of binding to M6903 and ABTIM3-h03, other huTIM-3 mutants resulted in loss of binding only to M6903, suggesting that the two antibodies have partially overlapping but distinct epitopes.

The other known antibodies, 27.12E12 and mab15, do not have hotspots revealed among this set of TIM-3 variant proteins despite competition observed in epitope binning experiments, suggesting that M6903 and ABTIM-3-mab15 have non-overlapping epitopes.

TABLE 4
Mutational scanning identifies hotspot residues in M6903 epitope

| | | 3903B11-IgG1 | | |
|---|---|---|---|---|
| | | (non-competitive | AB | AB |
| | M6903 | control) | TIM3-h03 | TIM3-mab15 |
| | K_{D} (nM) | | | |
| Ligand | | | | |
| hu TIM3 | 5.7 | 31 | 0.3 | 0.8 |
| P59A | ND† | 23 | 0.9*° | 0.6 |
| V60A | 3.7 | 23 | 0.4 | 0.5 |
| F61A | ND† | 27 | 0.6 | 0.8 |
| E62A | 88‡ | 19 | 0.4 | 0.5 |
| R111A | ND*† | 28 | 4.1*‡ | 3.0*° |
| R111E | ND*† | 99*° | 7.2*‡ | 2.4*° |
| I114A | 29*‡ | 27 | 0.7 | 0.6 |
| M118A | 10 | 30 | 0.7 | 1.1 |
| N119A | 17*° | 18 | 0.7 | 1.1 |
| K122A | 47*‡ | 36 | 8.4‡ | 1.8 |
| F123A | ND*† | 56 | 27† | 3.4° |

ΔΔGₘᵤₜ >2†; >1‡; >0.5° kcal/mol
ND = No binding detectable
*potential conformational destabilization or indirect contacts

### Example 2

### Pharmacology Studies for anti-TIM-3 Antibodies

The following studies refer to the anti-TIM3 antibody M6903. M6903 contains the light and heavy chain variable regions of 3903E11 (VL1.3,VH1.2) in an IgG2h(FN-AQ,322A)-delK background (anti-TIM3-3903E11(VL1.3,VH1.2)-IgG2h(FN-AQ,322A)-delK). The light and heavy chains of M6903 correspond to SEQ ID NO: 21 and SEQ ID NO: 22, respectively.

2.1 Target Occupancy of anti-TIM-3

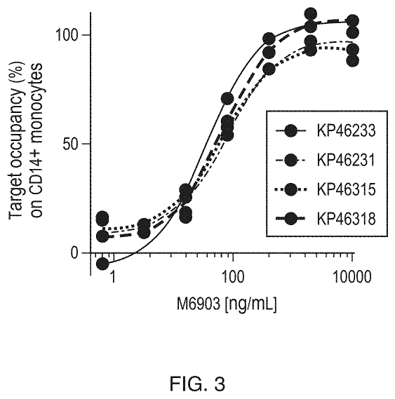

The ability of M6903 to bind to TIM-3 was demonstrated using anti-TIM-3 (A16-019-1), which is identical to M6903, but produced in Expi293F, not CHOK1SV, cells. The target occupancy of anti-TIM-3 (A16-019-1) on CD14+ monocytes was measured via flow cytometry using human whole blood samples. The samples were incubated with serial dilutions of anti-TIM-3 (A16-019-1) followed by anti-TIM-3(2E2)-APC, which has been shown to compete with anti-TIM-3 (A16-019-1) in binding to TIM-3 on CD14+ monocytes. As expected, target occupancy % increased with increasing concentrations of anti-TIM-3 (A16-019-1), and the average EC50 across all 10 donors was 111.1±85.6 ng/ml (see FIG. 3, which shows 4 representative donors (KP46233, KP46231, KP46315, and KP46318) out of the 10 total donors.). The highest doses were shown to saturate.

2.2 M6903 Efficiently Blocked the Interaction of rhTIM-3 and PtdSer on Apoptotic Jurkat Cells.

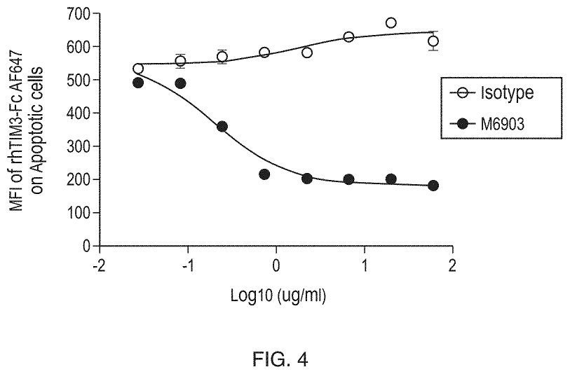

The ability of M6903 to block the interaction of TIM-3 with one if its ligands, PtdSer, was determined by a flow cytometry-based binding assay. Apoptotic Jurkat cells were used as the source for PtdSer, as the induction of apoptosis led to PtdSer exposure on the cell membrane of these cells. Specifically, prior to flow cytometry analysis, apoptosis was induced in Jurkat cells via treatment with Staurosporine (2 μg/mL, 18 hrs), leading to surface expression of a TIM-3 ligand, PtdSer. Binding of rhTIM-3-Fc PtdSer on the surface of apoptotic Jurkat cells was evaluated via flow cytometry by measuring the mean fluorescence intensity (MFI) of rhTIM-3-Fc AF647 after pre-incubation with serial dilutions of M6903 or an anti-HEL IgG2h isotype control. Pre-incubation of rhTIM-3 AF647 with M6903 led to reduced binding of TIM-3-Fc to apoptotic Jurkat cells, whereas pre-incubation with an isotype control had no effect on rhTIM-3-Fc binding (see FIG. 4). Therefore, M6903 was able to efficiently block the interaction between TIM-3 and PtdSer in a dose-dependent manner, with an IC50 of 4.438±3.115 nM (0.666±0.467 μg/mL). A nonlinear fit line was applied to the graph using a Sigmoid dose-response equation. It is hypothesized that this blockade of TIM-3/PtdSer interactions might lead to suppression of the inhibitory TIM-3 signaling and, as a result, enhanced immune cell activation.

2.3 Effect of M6903 on T Cell Recall Response and Activation as Monotherapy or in Combination with Bintrafusp Alfa

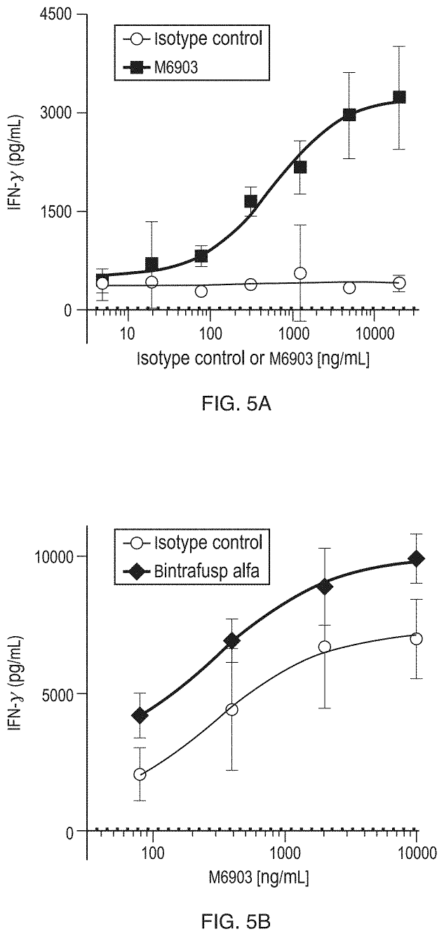

M6903 treatment increased IFN-γ production from human PBMCs that were activated by exposure to CEF antigens, which specifically elicits CEF antigen-specific T cell recall responses in the PBMCs from the donors who were previously infected with CEF. PBMCs were treated with 40 μg/ml CEF viral peptide pool for (A) 6 days or (B) 4 days in the presence of serial dilutions of M6903. As shown FIG. 5A, M6903 dose-dependently enhanced T cell activation compared to isotype control in a CEF assay as measured by IFN-γ production using a human IFN-γ ELISA kit, with an EC50 of 1±1.3 μg/mL, calculated from multiple experiments. Non-linear regression analysis was performed and mean and SD are presented.

As shown in FIG. 5B, serial dilutions of M6903 were combined with either 10 μg/mL isotype control or bintrafusp alfa. The production of IFN-γ was further enhanced in the presence of the combination of M6903 and bintrafusp alfa, suggesting that the combination might lead to further enhancement in T cell activation. Mean and SD are presented (p<0.05) in FIG. 5B.

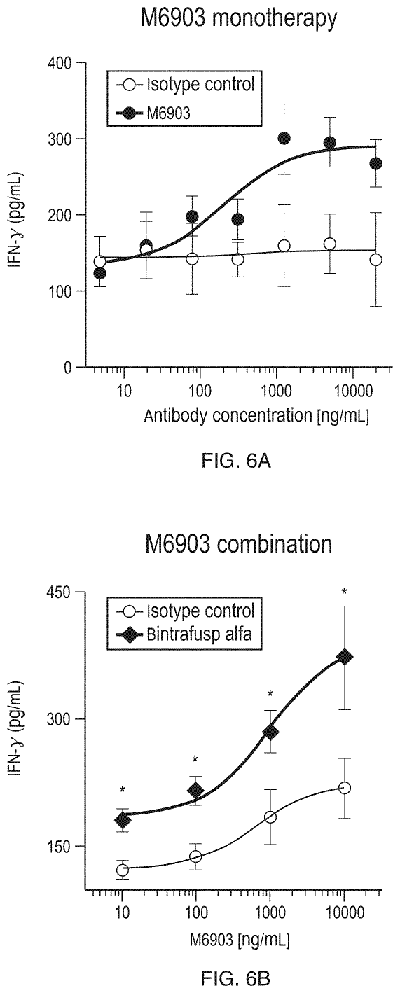

Irradiated Daudi tumor cells were co-cultured with human T cells for 7 days using IL-2 to induce allogenic reactive T cell expansion. The T cells were then harvested and co-cultured with freshly irradiated Daudi cells and treated with M6903 antibody or isotype control for 2 days. T cell activation was measured by an IFN-γ ELISA, and M6903 was shown to dose-dependently enhance IFN-γ production in these cells compared to the isotype control, with an EC50=116±117 ng/mL (sec FIG. 6A). The addition bintrafusp alfa further enhanced the effect of M6903 on T cell activation (see FIG. 6B).

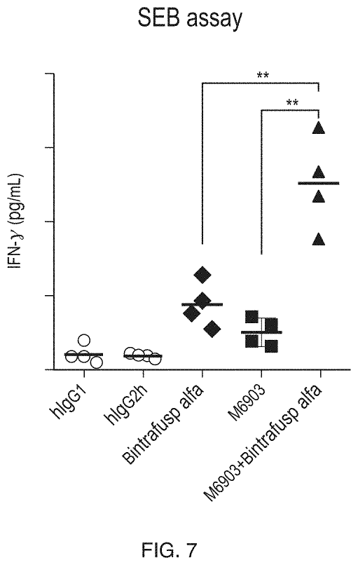

M6903 treatment increased IFN-γ production in human PBMCs that were activated by exposure to superantigen SEB, which activates CD4⁺ T cells non-specifically via cross-linking T cell receptor (TCR) and MHC class II molecules. M6903 (10 μg/mL) was incubated with 100 ng/mL SEB either alone or in combination with bintrafusp alfa (10 μg/mL) for 9 days, and cells were then washed once with medium and re-stimulated with 100 ng/mL SEB and antibody solutions with the same concentrations for an additional 2 days. Human IFN-γ in the supernatant was measured by using a human IFN-γ ELISA kit. M6903 treatment enhanced IFN-γ production (see FIG. 7). When M6903 treatment was combined with bintrafusp alfa, the production of IFN-γ was further enhanced (see FIG. 7).

2.4 Dual Blocking of Gal-9/PtdSer is Required to Potentiate T-Cell Activity, Correlating with M6903 Activity

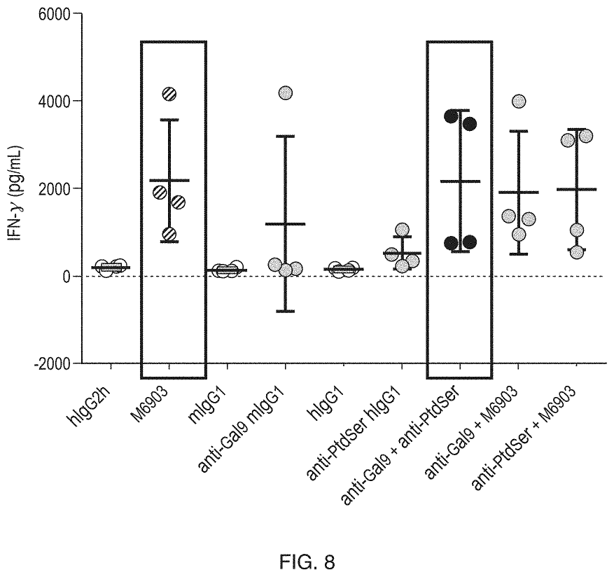

PBMCs were stimulated with 40 μg/ml CEF (Cytomegalovirus, Epstein Barr and Influenza) viral peptide pool (AnaSpec, AS-61036-025) for 4 days in AIM-V medium (Invitrogen #12055-091) with 5% human AB serum (Valley Biomedical, HP1022) in the presence of 10 μg/ml M6903, 10 μg/ml anti-Gal-9 (9M1-3; Biolegend, 348902), or 10 μg/ml anti-PtdSer (bavituximab; Creative Biolabs, TAB-175), or with antibody combinations 10 μg/ml M6903 and 10 μg/ml anti-Gal-9, 10 μg/ml M6903 and 10 μg/ml anti-PtdSer, or 10 μg/ml anti-Gal-9 and 10 μg/ml anti-PtdSer. Proliferation was measured by thymidine incorporation. IFN-γ in culture supernatant was measured by ELISA (R&D Systems, DY285B) and the results are shown in FIG. 8 (representative of at least 3 experiments; p<0.05. As shown, the combination of anti-Gal-9 and anti-PtdSer, but not either antibody alone, exhibited similar activity to M6903 in the CEF assay, suggesting that blocking the binding of both Gal-9 and PtdSer to TIM-3 might be required for anti-TIM-3 activity in this assay. In addition, the combination of M6903 with anti-Gal-9 or anti-PtdSer did not further increase IFNγ production, suggesting that M6903 fully blocked the binding of both Gal-9 and PtdSer to TIM-3.

2.5 Profiling TIM-3 Receptor and Ligand Expression in Normal Human Tissue and Tumors

Expression of TIM-3 and its ligands were then explored using chromogenic IHC and mIF validated assays. TIM-3 expression in normal human tissues was then evaluated using FDA normal tissue microarrays (TMA) representing 35 distinct tissues in the human body. Expression of TIM-3 was observed across most tissues and was specific to immune cells, except in the kidney cortex, where specific TIM-3 expression was also observed on epithelial cells. Highest immune reactivity was observed in immune tissues: spleen, tonsil, and lymph node, as well as in immune-rich organs: lung, placenta, and liver tissues. In immune organs, TIM-3 expression was primarily observed on macrophages (and possibly DCs) but not on lymphocytes (data not shown). TIM-3 expression on lymphocytes was observed only in inflamed tissue (data not shown).

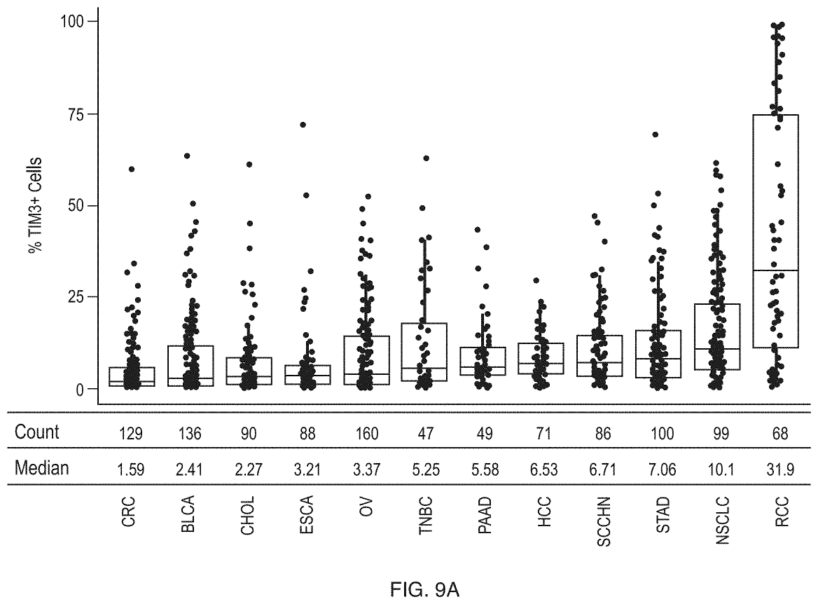

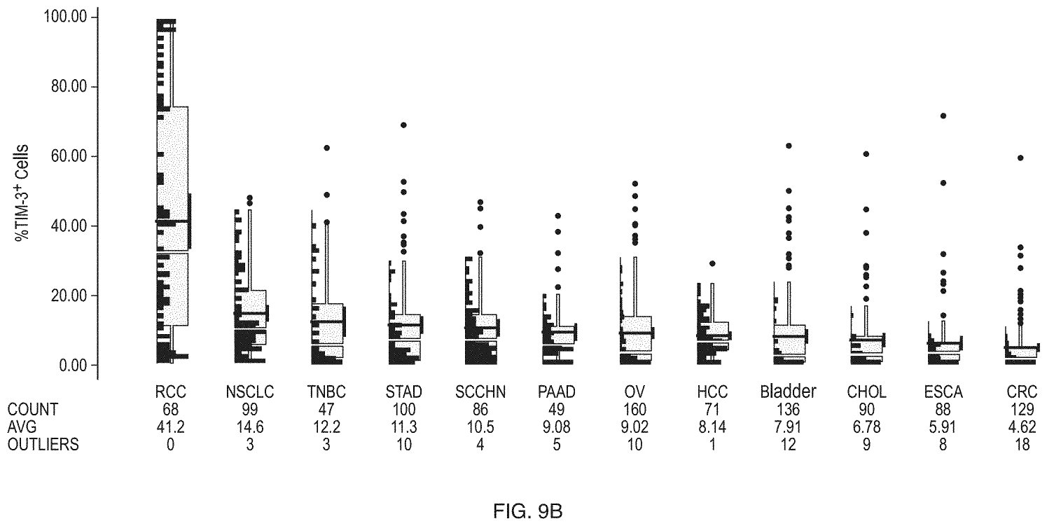

A review of the staining patterns across 15 tumor TMAs, representing 12 different tumor types, showed that TIM-3 expression was observed primarily on infiltrating immune cells across all indications except renal cell carcinoma (RCC). Phenotypically, both T cells and myeloid cells stained positive for TIM-3 (data not shown). Tumor cell expression of TIM-3 was seen only in RCC (data not shown). When the frequency of TIM-3⁺ cells was quantified using digital image analysis staining from these tumor TMAs, RCC showed the highest frequency of TIM-3 positivity (see FIGS. 9A and 9B), potentially due to the expression of TIM-3 on tumor cells in RCC, but not in other tumor types. The data were analyzed by (1) calculating mean expression and plotting the data by ascending median expression (FIG. 9A) and (2) calculating average expression following the removal of outliers and plotting the data by descending median expression (FIG. 9B). Other indications with high TIM-3 levels included NSCLC, stomach adenocarcinoma (STAD), triple negative breast cancer (TNBC) and squamous cell head and neck cancer (SCCHN) (see FIGS. 9A and 9B).

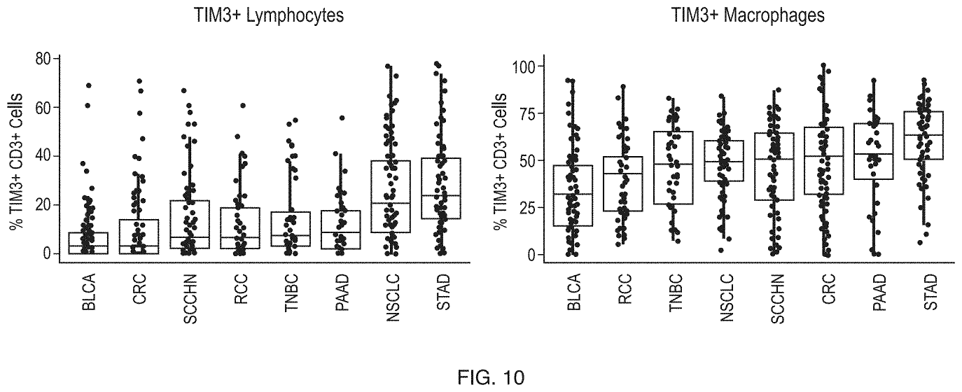

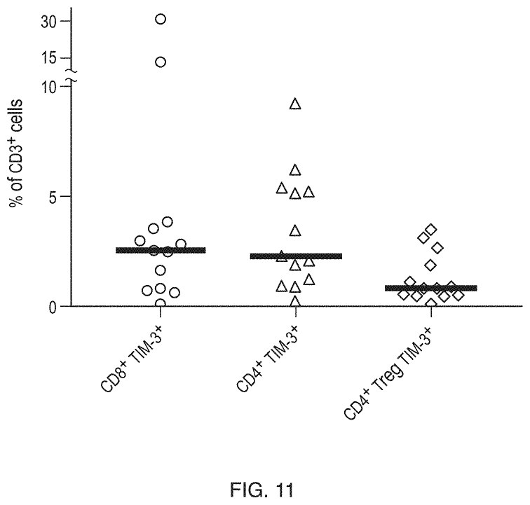

Tumor TMAs were then stained to identify immune cells expressing TIM-3 in the TME using mIF analysis. TIM-3 was found to be expressed on a subset of CD3⁺ lymphocytes and CD68⁺ macrophages. Digital quantitation showed that, while macrophages formed a significant fraction of TIM-3⁺ cells across all indications analyzed, a high frequency of TIM-3⁺ T cells were observed only in NSCLC and STAD tumors (see FIG. 10). These results were confirmed with flow cytometry analysis in a cohort of 13 NSCLC tumor samples; within the live CD3⁺ population, CD8⁺ T cells had the highest median percentage of TIM-3⁺ cells (5.126±2.331%), followed by CD4⁺ effector cells (3.398±0.732%), and CD4⁺ Tregs (1.316±0.310%) (see FIG. 11).

Finally, correlation of TIM-3 expression with ligands, Gal-9, CEACAM-1, and HMGB1, was evaluated both in the TCGA RNASeq data and mIF analyses (sec TABLE 5). Pearson correlation of TIM-3 expression with expression of ligands (mRNA and protein), showed that Gal-9 expression was positively correlated across multiple indications. This was not true for CEACAM-1 and HMGB1 expression. Values approaching 1 are the most positively correlated and those approaching −1 are the most negatively correlated, with values near 0 showing little to no correlation.

TABLE 5
Detection of TIM-3 and its ligands using mIF analysis

| | n | % GAL9⁺_area | % CEACAM⁺ | % HMGB1⁺ |
|---|---|---|---|---|
| SCLC | 6 | 0.95 | 0.88 | −0.91 |
| TNBC | 46 | 0.8 | −0.05 | 0.29 |
| Bladder | 25 | 0.79 | 0.6 | 0.25 |
| Melanoma | 22 | 0.79 | 0.52 | 0.46 |
| Breast | 46 | 0.68 | 0.7 | 0.42 |
| Endometrial | 28 | 0.59 | 0.43 | 0.53 |
| Lung | 35 | 0.57 | −0.25 | 0.09 |
| Ovarian | 25 | 0.52 | 0.25 | 0.13 |
| SCCHN | 75 | 0.44 | 0.21 | 0.27 |
| NSCLC | 97 | 0.41 | −0.03 | 0.07 |
| H&N | 32 | 0.4 | −0.36 | 0.02 |
| Prostate | 43 | 0.39 | −0.16 | −0.08 |
| Mouth | 17 | 0.36 | 0.09 | 0.44 |
| Kidney | 27 | 0.32 | −0.23 | 0.13 |
| Lymphoma | 43 | 0.32 | 0.17 | −0.21 |
| Gastric | 19 | 0.28 | −0.04 | 0.03 |
| Thyroid | 22 | 0.22 | −0.21 | 0.32 |
| Colon | 24 | −0.07 | −0.42 | −0.16 |

2.6 Explant Platform

Due to the lack of cross-reactivity of human TIM-3 protein with mouse TIM-3 protein, in vivo models are not readily available to interrogate the antitumor activity of M6903. Therefore, to determine whether M6903 had any anti-tumor efficacy, the CANscript™ human tumor microenvironment (TME) platform (developed at MITRA Biotech) was used. The CANscript™ platform is a functional assay that replicates a patient's personal tumor microenvironment, including the immune compartment. Responses to drug treatment applied to pieces of the tumor tissue in vitro are read out using multiple biochemical and phenotypic assays. These tumor responses are integrated by CANscript™ technology's algorithm into a single ‘M’-score that can predict efficacy of the drug.

Using this platform, M6903 was tested in samples from 20 patients with squamous cell carcinoma of the head and neck (SCCHN) either as monotherapy or in combination with bintrafusp alfa. The M-Score predicts treatment outcome based on multiple input parameters for the given tumor specimen. A positive prediction of response correlates to an M-Score greater than 25 (bold numbers in TABLE 6). A negative prediction of response correlates to an M-Score of 25 or lower. There are no M-Scores for the Control treatment as M-Score values are derived from parameters relative to the control untreated samples.

Using M-score as a readout of efficacy, positive predicted response was observed in 3/20 (15%) of tumor samples treated with M6903, 7/20 (35%) of tumor samples treated with bintrafusp alfa, and 9/20 (45%) of tumor samples treated with a combination of M6903 and bintrafusp alfa (see TABLE 6), suggesting that M6903 has anti-tumor activity which is increased in combination with bintrafusp.

TABLE 6
M-Score analysis for cumulative SCCHN tumors

| S. | Patient | Bintrafusp | | Bintrafusp |
|---|---|---|---|---|
| No | ID | alfa | M6903 | alfa + M6903 |
| 1 | HNS1 | 17 | 19 | 29 |
| 2 | HNS3 | 26 | 26 | 13 |
| 3 | HNS4 | 5 | 17 | 18 |
| 4 | HNS5 | 28 | 9 | 27 |
| 5 | HNS7 | 18 | 10 | 20 |
| 6 | HNS9 | 22 | 11 | 21 |
| 7 | HNS10 | 35 | 18 | 7 |
| 8 | HNS11 | 9 | 17 | 27 |
| 9 | HNS13 | 2 | 11 | 7 |
| 10 | HNS15 | 30 | 22 | 29 |
| 11 | HNS16 | 26 | 16 | 10 |
| 12 | HNS18 | 9 | 13 | 26 |
| 13 | HNS19 | 29 | 21 | 30 |
| 14 | HNS20 | 26 | 32 | 26 |
| 15 | HNS21 | 9 | 8 | 12 |
| 16 | HNS22 | 15 | 22 | 15 |
| 17 | HNS23 | 18 | 20 | 27 |
| 18 | HNS26 | 17 | 13 | 27 |
| 19 | HNS27 | 1 | 1 | 2 |
| 20 | HNS28 | 22 | 27 | 25 |

### Example 3

### In Vivo anti-TIM-3 Antibody Studies

3.1 Animals

A human TIM-3 knock-in mouse model was obtained from Beijing Biocytogen Co., Ltd, in which the murine extracellular domain of TIM-3 receptor was replaced with the human extracellular domain of TIM-3 receptor in a mouse C57BL/6 genetic background (“B-hu-TIM-3 KI” mice). B-hu-TIM-3 KI mice were generated using CRISPR/Cas9 recombination technology by replacing only the IgV extracellular domain (exon 2) of mouse with the corresponding human domain, which kept the remaining intracellular and cytoplasmic domains of the mouse TIM-3 receptor intact.

3.2 Anti-Tumor Efficacy of M6903/Bintrafusp Alfa in MC38 Tumor-Bearing B-huTIM-3 KI Mice

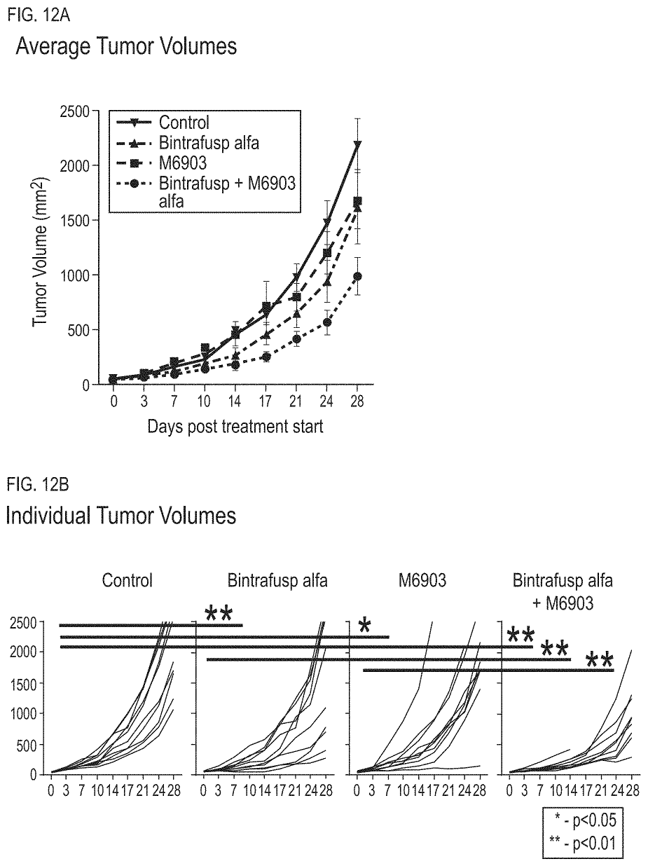

The antitumor efficacy effects of M6903 and bintrafusp alfa combination therapy were tested in a B-huTIM-3 KI mouse model subcutaneously implanted with MC38 tumors. 6-8 week old female mice (N=10/group) were treated with either isotype control (20 mg/kg; i.v; on days 0, 3, 6), bintrafusp alfa (24 mg/kg; i.v.; on days 0, 3, 6), M6903 (10 mg/kg; i.p.; q3dx12), or the combination of bintrafusp alfa and M6903. Significant anti-tumor activity was found with bintrafusp alfa monotherapy (TGI=25.7%, P=0.0054)) or with M6903 monotherapy (TGI=18.2%, P=0.0281) relative to isotype control, 28 days after the start of treatment (see FIG. 12A). Combination M6903 and bintrafusp alfa further enhanced anti-tumor activity (TGI=54.6%) relative to M6903 (P=0.0011, day 28) and bintrafusp alfa (P=0.0018, day 28) monotherapies (see FIGS. 12A, B). No significant treatment associated body weight loss was observed (data not shown).

### Example 4

### Clinical Study of the Combination of M6903 and Bintrafusp Alfa

4.1 Study Design

This is an exemplary single center, open-label, Phase I dose-escalation study investigating the safety, tolerability, pharmacokinetics, biological and clinical activity of the combination of M6903 and bintrafusp alfa in subjects with metastatic or advanced solid tumors that are relapsed/refractory or for which no standard therapy is available. Approximately 21-24 subjects (range 15-45) may be enrolled in this study. However, the total sample size will depend on the number of cohorts to be evaluated and the number of participants per cohort. The study will involve a total of five dose levels, with three dose levels with three subjects each and two dose levels with six subjects each, totaling 21 subjects. A Bayesian two-parameter logistic regression model will be applied to assist the safety monitoring committee (SMC) in dosing recommendations.

The study includes a screening period, a lead-in M6903 monotherapy and subsequent M6903 and bintrafusp alfa combination therapy treatment period and a follow-up period. M6903 and bintrafusp alfa are administered at a fixed rather than weight-based dose by intravenous infusion (IV) every two weeks. For M6903, the escalation doses are 20 mg (DL1), 80 mg (DL2), 240 mg (DL3), 800 mg (DL4) and 1600 mg (DL5). For bintrafusp alfa, the dose is 1200 mg. Each subject's DLT (dose limiting toxicity) period is six weeks (two weeks M6903 monotherapy lead-in followed by four weeks of combination therapy of M6903 and bintrafusp alfa). The subjects are treated until disease progression, unacceptable toxicity or removal of consent. Subjects will be followed for longer-term efficacy parameters such as PFS and OS if on active treatment or follow-up.

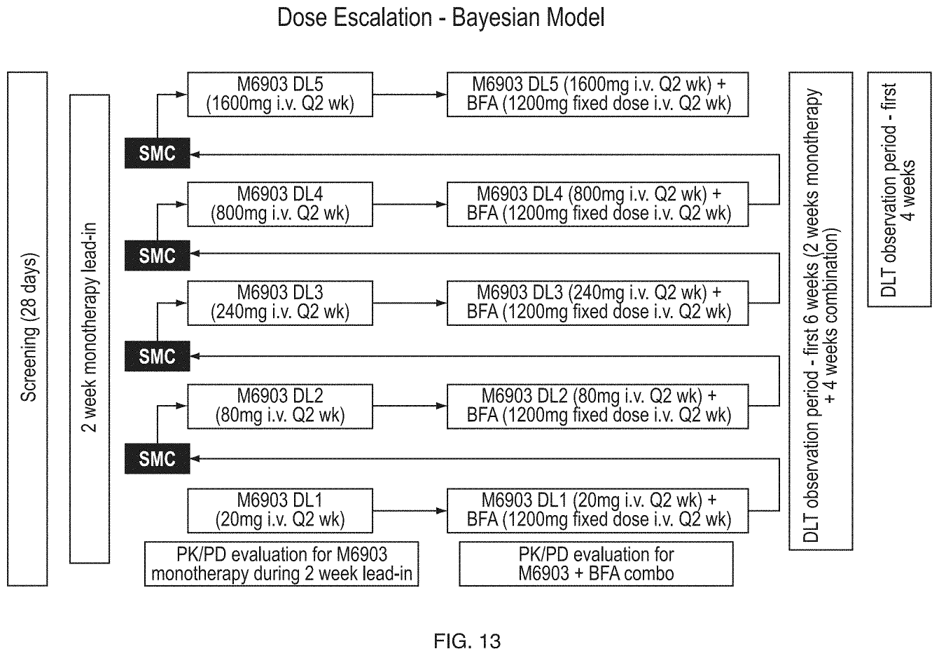

The dose escalation schema is presented in FIG. 13. As shown in FIG. 13, following a 28 day screening period, the subject is administered the M6903 escalation dose by IV infusion every two weeks. The two-week M6903 monotherapy lead-in period is followed by administration of the M6903 escalation dose in combination with 1200 mg of bintrafusp alfa (designated “BFA” in FIG. 13) by IV infusion every two weeks.

To characterize pharmacokinetic (PK) properties and pharmacodynamic responses to treatment, blood samples are taken at various time points during the M6903 monotherapy lead-in and the combination treatment of M6903 and bintrafusp alfa. M6903 PK parameters measured on Day 1, Day 15 and Day 43 are: AUCₗₐₛₜ, AUC_{0-∞}, AUC_{τ}, Cₘₐₓ, Cₚᵣₑ, Tₘₐₓ, t_{1/2} and terminal rate constant. Further assessments are presented in TABLE 7 (Schedule of Assessments).

4.2 Study Objectives

The primary objectives of this study are to evaluate the safety and tolerability of M6903 and to determine the recommended expansion dose of M6903 for expansion studies.

The secondary objectives are as follows:
To characterize PK profile of M6903;
To characterize the peripheral TIM3 target occupancy (TO) with M6903 alone and exposure/target occupancy relationship;
To characterize immunogenicity of M6903;
To assess concentration-QTcF relationship using central ECG; and
To evaluate preliminary efficacy parameters (PFS, BOR, DOR) using RECIST v1.1.

The exploratory objectives are as follows:
To evaluate overall survival;
To evaluate the effect of M6903 on immune cell subsets and soluble factors in blood;

and
To evaluate the effect of M6903 in the tumor.

4.3 Study Population

Subjects must meet the following key inclusion criteria for study entry:
1. Subjects who are ≥18 years of age inclusive, at the time of signing the informed consent;
2. Histologically or cytologically proven metastatic or locally advanced solid tumors that is measurable as per RECIST v1.1, for which no standard therapy exists or relapsed/refractory from at least 1 prior treatment;
3. ECOG performance status of 0 to 1; and
4. Adequate renal, hepatic, and hematologic function.

In addition, subjects who meet any of the following exclusion criteria are excluded from study entry:
1. Previous malignant disease (other than the tumor disease for this trial) within the last 2 years (except adequately treated non-melanoma skin cancers and carcinoma in situ located in the skin, bladder, cervix, colon/rectum, breast, or prostate) unless a complete remission without further recurrence was achieved at least 1 years prior to study entry and the subject was deemed to have been cured with no additional therapy required or anticipated to be required.
2. Active autoimmune disease that might deteriorate when receiving an immunostimulatory agent. Subjects with type I diabetes mellitus, vitiligo, psoriasis, hypo- or hyperthyroid disease not requiring immunosuppressive treatment are eligible. Please consult with a Medical Monitor for cases of uncertainty prior to signing informed consent.
3. Persisting toxicity related to prior therapy (NCI-CTCAE v4.03 Grade >1); however, alopecia, sensory neuropathy Grade ≤2, or other Grade ≤2 AEs not constituting a safety risk based on investigator's judgment are acceptable.
4. Current use of the following medications at the time of enrollment:
   a. Immunosuppressive drugs (e.g., chemotherapy or systemic corticosteroids) EXCEPT for the following: (i) intranasal, inhaled, topical steroids, or local steroid injection (eg, intra-articular injection); (ii) systemic corticosteroids at physiologic doses ≤10 mg/day of prednisone or equivalent; (iii) steroids as premedication for hypersensitivity reactions (e.g., CT scan premedication);
   b. Growth factors (granulocyte colony stimulating factor or granulocyte macrophage colony stimulating factor);
   c. Herbal remedies with immunostimulating properties (e.g., mistletoe extract) or known to potentially interfere with major organ function (e.g., hypericin).
5. All subjects with brain metastases, except those meeting the following criteria:
   a. Brain metastases that have been treated locally and are clinically stable for at least 2 weeks prior to randomization;
   b. No ongoing neurological symptoms that are related to the brain localization of the disease (sequelae that are a consequence of the treatment of the brain metastases are acceptable);
   c. Subjects must be either off steroids or on a stable or decreasing dose of <10 mg daily prednisone (or equivalent).
6. Prior organ transplantation, including allogeneic stem cell transplantation.
7. Hepatitis B virus (HBV) or hepatitis C virus (HCV) infection at screening (positive HBV surface antigen or HCV RNA if anti-HCV antibody screening test positive).

TABLE 7
Schedule of Assessment (week 1-2 are mono therapy only; week 3 and beyond
are combination of M6903 plus bintrafusp alfa)

| | | | | | | | | | | | | | | Discontinuation/ | | |
|---|---|---|---|---|---|---|---|---|---|---|---|---|---|---|---|---|
| | | | | | | | | | | | | | | End of | Safety | Long-term |
| | Screen | Monotherapy | | | | Combination therapy | | | | | | | | Treatment | Follow-up | Follow-up |
| | Week | | | | | | | | | | | | | | | |
| | | | | | | | | | | | | | | Up to 7/28 | | |
| | | | | | | | | | | | | | Until | Days | 10 weeks | |
| | | | | | | | | | | | | | PD | after Last | after Last | Every 3 |
| | −4 to 0 | 1 | | | 2 | 3 | 4 | 5 | 7 | 9 | 11 | 13 | (Weeks) | Treatment | Treatment | months |
| | Study Day | | | | | | | | | | | | | | | |
| | −28 to 0 | 1 | 2 | 5 | 8 | 15 | 22 | 29 | 43 | 57 | 71 | 85 | | | | |
| | Visit Window (d) | | | | | | | | | | | | | | | |
| | | | | | | | | | | | | | −3/1 | | | |
| | | | | | ± 1 | ± 1 | ± 1 | ± 1 | ± 1 | −3/1 | −3/1 | −3/1 | (days) | ± 5 | ± 2 weeks | |
| M6903 | | X | | | | X | | X | | X | X | X | 2 | | | |
| Bintrafusp alfa | | | | | | X | | X | | X | X | X | 2 | | | |
| Written informed | X | | | | | | | | | | | | | | | |
| consent | | | | | | | | | | | | | | | | |
| Demographics, | X | | | | | | | | | | | | | | | |
| height | | | | | | | | | | | | | | | | |
| Medical history | X | | | | | | | | | | | | | | | |
| Serum/urine | X | | | | | | | X | | | | | 4 | | X | |
| pregnancy test (if | | | | | | | | | | | | | | | | |
| applicable) | | | | | | | | | | | | | | | | |
| Physical | X | X | | | | X | X | X | X | | | | 2 | X | X | |
| examination, | | | | | | | | | | | | | | | | |
| temperature | | | | | | | | | | | | | | | | |
| Weight | X | X | | | | X | | X | | X | X | X | 2 | | | |
| ECOG performance | X | X | | | | X | X | X | X | | | | 2 | X | X | |
| status | | | | | | | | | | | | | | | | |
| Vital signs | X | X | | | | X | X | X | X | | | | 2 | X | X | |
| HBV, HCV, HIV^{j} | X | | | | | | | | | | | | | | | |
| testing (if applicable) | | | | | | | | | | | | | | | | |
| AE assessment | X | X | | | | X | X | X | X | | | | 2 | X | X | |
| DLT assessment | | | | | | | X | | | | | | | | | |
| Medication history | X | | | | | | | | | | | | | | | |
| Concomitant | X | X | | | | X | | X | X | | | | 2 | X | XX | |
| medications | | | | | | | | | | | | | | | | |
| 12-lead ECG | X | X/X | X | | X | X/X | | X | X/X | | | X | 6 | X | X | |
| Hematology, serum | X | X | | | | X | X | X | X | | | | 2 | X | X | |
| chemistry, | | | | | | | | | | | | | | | | |
| Coagulation | X | | | | | | | | | | | | | | | |
| Urinalysis dipstick^{p} | X | | | | | | | X | | | | | 12 | X | X | |
| Free T4, TSH | X | | | | | | | X | | | | | 6 | X | X | |
| Tumor biopsy or | X | | | | | | | | | | | | | | | |
| archived surgical | | | | | | | | | | | | | | | | |
| specimen (optional) | | | | | | | | | | | | | | | | |
| Tumor assessment^{q} | | | | | | | | | | X | | | Q8 wks | X^{s} | | X^{t} |
| | | | | | | | | | | | | | for first | | | |
| | | | | | | | | | | | | | year, then | | | |
| | | | | | | | | | | | | | Q12 wks | | | |

| | |
|---|---|
| Survival | X |

Blood sampling

Abbreviations: AE = adverse event, ECG = electrocardiogram, ECOG = Eastern Cooperative Oncology Group, EoT = End of Treatment visit, EOT = End of Trial visit, FFPE = formalin fixed and paraffin embedded, HBV = Hepatitis B virus, HCV = Hepatitis C virus, HIV = Human immunodeficiency virus, IMP = Investigational medicinal product, PK = pharmacokinetic, RECIST = Response Evaluation Criteria in Solid Tumors.
Notes:
If another antineoplastic therapy is administered before the end of the 28-day period, the End-of-Treatment visit should be conducted before the start of new therapy if possible Subjects without progressive disease at End-of-Treatment visit will be followed up for disease progression (CT/MRI scans every 6 weeks) until PD and/or the start of a new treatment. After completion of the Follow-up period the appropriate electronic Case Report Form section for Trial Termination must be completed.

### INCORPORATION BY REFERENCE

The entire disclosure of each of the patent and scientific documents referred to herein is incorporated by reference for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING:

SEQ
ID
NO

Anti-TIM3 Antibodies: Optimization

| | |
|---|---|
| | CDRH1 3903E11/M6903 |
| 1 | GFTFSSYA |
| | CDRH2 3903E11/M6903 |
| 2 | ISVSGGST |
| | CDRH3 3903E11/M6903 |
| 3 | AKANWGFFDY |
| | CDRL1 3903E11/M6903 |
| 4 | SSDVGGYNY |
| | CDRL2 3903E11/M6903 |
| 5 | DVS |
| | CDRL3 3903E11/M6903 |
| 6 | SSYADSW |
| | FR-1 of the heavy chain of |
| | antibody 3903E11 family, |
| | with X being any residues |
| | selected from the group |
| | consisting of Q |
| | (glutamine) and E |
| | (glutamic acid) |
| 7 | EVQLVXSGGGLVQPGGSLRLSCAAS |
| | FR-2 of the heavy chain of |
| | antibody 3903E11 family, |
| | with X being any residues |
| | selected from the group |
| | consisting of M |
| | (methionine) and L (leucine) |
| 8 | XSWVRQAPGKGLEWVSA |
| | FR-3 of the heavy chain of |
| | antibody 3903E11 family. |
| 9 | YYADSVKGRFTISRDNSKNTLYL |
| | QMNSLRAEDTAVYYC |
| | FR-4 of the heavy chain of |
| | antibody 3903E11 family |
| 10 | WGQGTLVTVSS |
| | FR-1 of the light chain of |
| | antibody 3903E11 family, |
| | with X₁ being any residues |
| | selected from the group |
| | consisting of S (serine) |
| | and Q (glutamine), X₂ |
| | being any residues selected |
| | from the group consisting |
| | of Y (tyrosine) and |
| | S (serine) and |
| | X₃ being E (glutamic acid) |
| | and A (alanine) |
| 11 | X₁X₂X₃LTQPRSVSGS |
| | PGQSVTISCTGT |
| | FR-2 of the light chain of |
| | antibody 3903E11 family, |
| | with X being any residues |
| | selected from the group |
| | consisting of F |
| | (phenylalanine) and Y |
| | (tyrosine) |
| 12 | VSWYQQHPGKAPKLMIX |
| | FR-3 of the light chain |
| | of antibody 3903E11 family |
| 13 | KRPSGVPDRFSGSKSGNTASLT |
| | ISGLQAEDEADYYC |
| | FR-4 of the light chain |
| | of antibody 3903E11 family |
| 14 | FGGGTKVTVL |
| | Light Chain Variant 1 |
| | 3903E11 (VL1.1)-CL |
| 15 | QSALTQPRSVSGSPGQSVTISCTGTSSDVGG |
| | YNYVSWYQQHPGKAPKLMIFDVSKRPSGVPD |
| | RFSGSKSGNTASLTISGLQAEDEADYYCSSY |
| | ADSVVFGGGTKVTVLGQPKAAPSVTLFPPSS |
| | EELQANKATLVCLISDFYPGAVTVAWKADSS |
| | PVKAGVETTTPSKQSNNKYAASSYLSLTPEQ |
| | WKSHKSYSCQVTHEGSTVEKTVAPTECS |
| | Light Chain Variable |
| | Region 3903E11 (VL1.1) |
| 52 | QSALTQPRSVSGSPGQSVTISCTGTSSDVGG |
| | YNYVSWYQQHPGKAPKLMIFDVSKRPSGVPD |
| | RFSGSKSGNTASLTISGLQAEDEADYYCSSY |
| | ADSVVFGGGTKVTVL |
| | Heavy Chain Variant 1 |
| | 3903E11 (VH1.1)-gl |
| 16 | EVQLVQSGGGLVQPGGSLRLSCAASGFTFSS |
| | YALSWVRQAPGKGLEWVSAISVSGGSTYYAD |
| | SVKGRFTISRDNSKNTLYLQMNSLRAEDTAV |
| | YYCAKANWGFFDYWGQGTLVTVSSASTKGPS |
| | VFPLAPSSKSTSGGTAALGCLVKDYFPEPVT |
| | VSWNSGALTSGVHTFPAVLQSSGLYSLSSVV |
| | TVPSSSLGTQTYICNVNHKPSNTKVDKRVEP |
| | KSCDKTHTCPPCPAPELLGGPSVFLFPPKPK |
| | DTLMISRTPEVTCVVVDVSHEDPEVKFNWYV |
| | DGVEVHNAKTKPREEQYNSTYRVVSVLTVLH |
| | QDWLNGKEYKCKVSNKALPAPIEKTISKAKG |
| | QPREPQVYTLPPSREEMTKNQVSLTCLVKGF |
| | YPSDIAVEWESNGQPENNYKTTPPVLDSDGS |
| | FFLYSKLTVDKSRWQQGNVFSCSVMHEALHN |
| | HYTQKSLSLSPG |
| | Heavy Chain Variable Region |
| | 3903E11 (VH1.1) |
| 53 | EVQLVQSGGGLVQPGGSLRLSCAASGFTFSS |
| | YALSWVRQAPGKGLEWVSAISVSGGSTYYAD |
| | SVKGRFTISRDNSKNTLYLQMNSLRAEDTAV |
| | YYCAKANWGFFDYWGQGTLVTVSS |
| | Light Chain Variant 2 3903E11 |
| | (VL1.2)-CL |
| 17 | SyeLTQPRSVSGSPGQSVTISCTGTSSDVGG |
| | YNYVSWYQQHPGKAPKLMIYDVSKRPSGVPD |
| | RFSGSKSGNTASLTISGLQAEDEADYYCSSY |
| | ADSVVFGGGTKVTVLGQPKAAPSVTLFPPSS |
| | EELQANKATLVCLISDFYPGAVTVAWKADSS |
| | PVKAGVETTTPSKQSNNKYAASSYLSLTPEQ |
| | WKSHKSYSCQVTHEGSTVEKTVAPTECS |
| | Light Chain Variable Region 3903E11 |
| | (VL1.2) |
| 54 | SyeLTQPRSVSGSPGQSVTISCTGTSSDVGG |
| | YNYVSWYQQHPGKAPKLMIYDVSKRPSGVPD |
| | RFSGSKSGNTASLTISGLQAEDEADYYCSSY |
| | ADSWFGGGTKVTVL |
| | Heavy Chain Variant 2 3903E11 |
| | (VH1.2)-g1 |
| 18 | EVQLVESGGGLVQPGGSLRLSCAASGFTFSS |
| | YAMSWVRQAPGKGLEWVSAISVSGGSTYYAD |
| | SVKGRFTISRDNSKNTLYLQMNSLRAEDTAV |
| | YYCAKANWGFFDYWGQGTLVTVSSASTKGPS |
| | VFPLAPSSKSTSGGTAALGCLVKDYFPEPVT |
| | VSWNSGALTSGVHTFPAVLQSSGLYSLSSVV |
| | TVPSSSLGTQTYICNVNHKPSNTKVDKRVEP |
| | KSCDKTHTCPPCPAPELLGGPSVFLFPPKPK |
| | DTLMISRTPEVTCVVVDVSHEDPEVKFNWYV |
| | DGVEVHNAKTKPREEQYNSTYRVVSVLTVLH |
| | QDWLNGKEYKCKVSNKALPAPIEKTISKAKG |
| | QPREPQVYTLPPSREEMTKNQVSLTCLVKGF |
| | YPSDIAVEWESNGQPENNYKTTPPVLDSDGS |
| | FFLYSKLTVDKSRWQQGNVFSCSVMHEALHN |
| | HYTQKSLSLSPG |
| | Heavy Chain Variable Region |
| | 3903E11 (VH1.2) |
| 24 | EVQLVESGGGLVQPGGSLRLSCAASGFTFSS |
| | YAMSWVRQAPGKGLEWVSAISVSGGSTYYAD |
| | SVKGRFTISRDNSKNTLYLQMNSLRAEDTAV |
| | YYCAKANWGFFDYWGQGTLVTVSS |
| | Light Chain Variant 3 |
| | 3903E11 (VL1.3)-CL |
| 19 | QSALTQPRSVSGSPGQSVTISCTGTSSDVGG |
| | YNYVSWYQQHPGKAPKLMIFDVSKRPSGVPD |
| | RFSGSKSGNTASLTISGLQAEDEADYYCSSY |
| | ADSVVFGGGTKVTVLGQPKAAPSVTLFPPSS |
| | EELQANKATLVCLISDFYPGAVTVAWKADSS |
| | PVKAGVETTTPSKQSNNKYAASSYLSLTPEQ |
| | WKSHKSYSCQVTHEGSTVEKTVAPTECS |
| | Light Chain Variable |
| | Region 3903E11 (VL1.3) |
| 23 | QSALTQPRSVSGSPGQSVTISCTGTSSDVGG |
| | YNYVSWYQQHPGKAPKLMIFDVSKRPSGVPD |
| | RFSGSKSGNTASLTISGLQAEDEADYYCSSY |
| | ADSWFGGGTKVTVL |
| | Heavy Chain Variant 3 3903E11 |
| | (VH1.3)-g1 |
| 20 | EVQLVESGGGLVQPGGSLRLSCAASGFTFSS |
| | YALSWVRQAPGKGLEWVSAISVSGGSTYYAD |
| | SVKGRFTISRDNSKNTLYLQMNSLRAEDTAV |
| | YYCAKANWGFFDYWGQGTLVTVSSASTKGPS |
| | VFPLAPSSKSTSGGTAALGCLVKDYFPEPVT |
| | VSWNSGALTSGVHTFPAVLQSSGLYSLSSVV |
| | TVPSSSLGTQTYICNVNHKPSNTKVDKRVEP |
| | KSCDKTHTCPPCPAPELLGGPSVFLFPPKPK |
| | DTLMISRTPEVTCVVVDVSHEDPEVKFNWYV |
| | DGVEVHNAKTKPREEQYNSTYRVVSVLTVLH |
| | QDWLNGKEYKCKVSNKALPAPTEKTISKAKG |
| | QPREPQVYTLPPSREEMTKNQVSLTCLVKGF |
| | YPSDIAVEWESNGQPENNYKTTPPVLDSDGS |
| | FFLYSKLTVDKSRWQQGNVFSCSVMHEALHN |
| | HYTQKSLSLSPG |
| | Heavy Chain Variable Region |
| | 3903E11 (VH1.3) |
| 55 | EVQLVESGGGLVQPGGSLRLSCAASGFTFSS |
| | YALSWVRQAPGKGLEWVSAISVSGGSTYYAD |
| | SVKGRFTISRDNSKNTLYLQMNSLRAEDTAV |
| | YYCAKANWGFFDYWGQGTLVTVSS |

M6903 (anti-TIM3-3903E11
(VL1.3, VH1.2)-huIgG2h (FN-AQ,
K322A)-delK): Amino Acid

| | |
|---|---|
| | Light Chain |
| 21 | QSALTQPRSVSGSPGQSVTISCTGTSSDVGG |
| | YNYVSWYQQHPGKAPKLMIYDVSKRPSGVPD |
| | RFSGSKSGNTASLTISGLQAEDEADYYCSSY |
| | ADSVVFGGGTKVTVLGQPKAAPSVTLFPPSS |
| | EELQANKATLVCLISDFYPGAVTVAWKADSS |
| | PVKAGVETTTPSKQSNNKYAASSYLSLTPEQ |
| | WKSHKSYSCQVTHEGSTVEKTVAPTECS |
| | Heavy Chain |
| 22 | EVQLVESGGGLVQPGGSLRLSCAASGFTFSS |
| | YAMSWVRQAPGKGLEWVSAISVSGGSTYYAD |
| | SVKGRFTISRDNSKNTLYLQMNSLRAEDTAV |
| | YYCAKANWGFFDYWGQGTLVTVSSASTKGPS |
| | VFPLAPCSRSTSESTAALGCLVKDYFPEPVT |
| | VSWNSGALTSGVHTFPAVLQSSGLYSLSSVV |
| | TVPSSNFGTQTYTCNVDHKPSNTKVDKTVEP |
| | KSSDKTHTCPPCPAPPVAGPSVFLFPPKPKD |
| | TLMISRTPEVTCVVVDVSHEDPEVQFNWYVD |
| | GVEVHNAKTKPREEQAQSTFRVVSVLTVVHQ |
| | DWLNGKEYKCAVSNKGLPAPIEKTISKTKGQ |
| | PREPQVYTLPPSREEMTKNQVSLTCLVKGFY |
| | PSDIAVEWESNGQPENNYKTTPPMLDSDGSF |
| | FLYSKLTVDKSRWQQGNVFSCSVMHEALHNH |
| | YTQKSLSLSPG |
| | Light Chain Variable Region (VL1.3) |
| 23 | QSALTQPRSVSGSPGQSVTISCTGTSSDVGGY |
| | NYVSWYQQHPGKAPKLMIYDVSKRPSGVPDRF |
| | SGSKSGNTASLTISGLQAEDEADYYCSSYADS |
| | WFGGGTKVTVL |
| | Heavy Chain Variable Region |
| | (VH1.2) |
| 24 | EVQLVESGGGLVQPGGSLRLSCAASGFTFSSY |
| | AMSWVRQAPGKGLEWVSAISVSGGSTYYADSV |
| | KGRFTISRDNSKNTLYLQMNSLRAEDTAVYYC |
| | AKANWGFFDYWGQGTLVTVSS |
| | Light Chain Constant Region |
| 25 | GQPKAAPSVTLFPPSSEELQANKATLVCLISD |
| | FYPGAVTVAWKADSSPVKAGVETTTPSKQSNN |
| | KYAASSYLSLTPEQWKSHKSYSCQVTHEGSTV |
| | EKTVAPTECS |
| | Heavy Chain Constant Region |
| 26 | ASTKGPSVFPLAPCSRSTSESTAALGCLVKDY |
| | FPEPVTVSWNSGALTSGVHTFPAVLQSSGLYS |
| | LSSVVTVPSSNFGTQTYTCNVDHKPSNTKVDK |
| | TVEPKSSDKTHTCPPCPAPPVAGPSVFLFPPK |
| | PKDTLMISRTPEVTCVVVDVSHEDPEVQFNWY |
| | VDGVEVHNAKTKPREEQAQSTFRVVSVLTVVH |
| | QDWLNGKEYKCAVSNKGLPAPIEKTISKTKGQ |
| | PREPQVYTLPPSREEMTKNQVSLTCLVKGFYP |
| | SDIAVEWESNGQPENNYKTTPPMLDSDGSFFL |
| | YSKLTVDKSRWQQGNVFSCSVMHEALHNHYTQ |
| | KSLSLSPG |

M6903 (anti-TIM3-3903E11 (VL1.3, VH1.2)-
huIgG2h (FN-AQ, K322A)-delK): Nucleotide

| | |
|---|---|
| | Light Chain Variable |
| 27 | CAGAGCGCCCTGACACAGCCTCGCTCAGTGTC |
| | CGGGTCTCCTGGACAGTCAGTCACCATCTCCT |
| | GCACTGGAACCAGCAGTGATGTTGGTGGTTAT |
| | AACTATGTCTCCTGGTACCAACAGCACCCAGG |
| | CAAAGCCCCCAAACTCATGATTTACGATGTCA |
| | GTAAGCGGCCCTCAGGGGTCCCTGATCGCTTC |
| | TCTGGCTCCAAGTCTGGCAACACGGCCTCCCT |
| | GACCATCTCTGGGCTCCAGGCTGAGGATGAGG |
| | CTGATTATTACTGCTCCTCATATGCAGACAGC |
| | GTGGTATTCGGCGGAGGGACCAAGGTGACCGT |
| | CCTAGG |
| | Heavy Chain Variable |
| 28 | GAGGTGCAGCTGGTGGAGTCTGGGGGAGGCTT |
| | GGTACAGCCTGGGGGGTCCCTGAGACTCTCCT |
| | GTGCAGCCTCTGGATTCACCTTTAGCAGCTAT |
| | GCCATGAGCTGGGTCCGCCAGGCTCCAGGGAA |
| | GGGGCTGGAGTGGGTCTCAGCTATTAGTGTTA |
| | GTGGTGGTAGCACATACTACGCAGACTCCGTG |
| | AAGGGCCGATTCACCATCTCCAGAGACAATTC |
| | CAAGAACACGCTGTATCTGCAAATGAACAGCC |
| | TGAGAGCCGAGGACACGGCCGTATATTACTGT |
| | GCGAAAGCCAACTGGGGGTTCTTTGACTACTG |
| | GGGCCAGGGAACCCTGGTCACTGTCTCTTCA |
| | Light Chain Constant |
| 29 | GGACAGCCCAAGGCTGCCCCCTCGGTCACTCT |
| | GTTCCCGCCCTCCTCTGAGGAGCTTCAAGCCA |
| | ACAAGGCCACACTGGTGTGTCTCATAAGTGAC |
| | TTCTACCCGGGAGCCGTGACAGTGGCCTGGAA |
| | GGCAGATAGCAGCCCCGTCAAGGCGGGAGTGG |
| | AGACCACCACACCCTCCAAACAAAGCAACAAC |
| | AAGTACGCGGCCAGCAGCTACCTGAGCCTGAC |
| | GCCTGAGCAGTGGAAGTCCCACAAAAGCTACA |
| | GCTGCCAGGTCACGCATGAAGGGAGCACCGTG |
| | GAGAAGACAGTGGCCCCTACAGAATGTTCA |
| | Heavy Chain Constant |
| 30 | GCTAGCACCAAGGGCCCATCGGTCTTCCCCCT |
| | GGCGCCCTGCTCCAGGAGCACCTCCGAGAGCA |
| | CAGCGGCCCTGGGCTGCCTGGTCAAGGACTAC |
| | TTCCCCGAACCGGTGACGGTGTCGTGGAACTC |
| | AGGCGCTCTGACCAGCGGCGTGCACACCTTCC |
| | CAGCTGTCCTACAGTCCTCAGGACTCTACTCC |
| | CTCAGCAGCGTGGTGACCGTGCCCTCCAGCAA |
| | CTTCGGCACCCAGACCTACACCTGCAACGTAG |
| | ATCACAAGCCCAGCAACACCAAGGTGGACAAG |
| | ACAGTTGAGCCCAAATCTTCTGACAAAACTCA |
| | CACATGCCCACCGTGCCCAGCACCACCTGTGG |
| | CAGGACCGTCAGTCTTCCTCTTCCCCCCAAAA |
| | CCCAAGGACACCCTCATGATCTCCCGGACCCC |
| | TGAGGTCACGTGCGTGGTGGTGGACGTGAGCC |
| | ACGAAGACCCCGAGGTCCAGTTCAACTGGTAC |
| | GTGGACGGCGTGGAGGTGCATAATGCCAAGAC |
| | AAAGCCACGGGAGGAGCAGGCCCAGAGCACGT |
| | TCCGTGTGGTCAGCGTCCTCACCGTTGTGCAC |
| | CAGGACTGGCTGAACGGCAAGGAGTACAAGTG |
| | CGCTGTCTCCAACAAAGGCCTCCCAGCCCCCA |
| | TCGAGAAAACCATCTCCAAAACCAAAGGGCAG |
| | CCCCGAGAACCACAGGTGTACACCCTGCCCCC |
| | ATCACGGGAGGAGATGACCAAGAACCAGGTCA |
| | GCCTGACCTGCCTGGTCAAAGGCTTCTACCCC |
| | AGCGACATCGCCGTGGAGTGGGAGAGCAATGG |
| | GCAGCCGGAGAACAACTACAAGACCACACCTC |
| | CCATGCTGGACTCCGACGGCTCCTTCTTCCTC |
| | TACAGCAAGCTCACCGTGGACAAGAGCAGGTG |
| | GCAGCAGGGGAACGTCTTCTCATGCTCCGTGA |
| | TGCATGAGGCTCTGCAC |
| | AACCACTACACACAGAAGAGCCTCTCCCTGTC |
| | CCCGGGT |
| | Parental Antibody 3903E11 |
| | (VL1.0, VH1.0): Amino Acid |
| | Light Chain |
| 31 | syeLTQPRSVSGSPGQSVTISCTGTSSDVGGYN |
| | YVSWYQQHPGKAPKLMIFDVSKRPSGVPDRFSG |
| | SKSGNTASLTISGLQAEDEADYYCSSYADSVVF |
| | GGGTKVTVLGQPKAAPSVTLFPPSSEELQANK |
| | ATLVCLISDFYPGAVTVAWKADSSPVKAGVET |
| | TTPSKQSNNKYAASSYLSLTPEQWKSHKSYSC |
| | QVTHEGSTVEKTVAPTECS |
| | Heavy Chain |
| 32 | EVQLVQSGGGLVQPGGSLRLSCAASGFTFSSY |
| | AMSWVRQAPGKGLEWVSAISVSGGSTYYADSV |
| | KGRFTISRDNSKNTLYLQMNSLRAEDTAVYYC |
| | AKANWGFFDYWGQGTLVTVSSASTKGPSVFPL |
| | APSSKSTSGGTAALGCLVKDYFPEPVTVSWNS |
| | GALTSGVHTFPAVLQSSGLYSLSSVVTVPSSS |
| | LGTQTYICNVNHKPSNTKVDKRVEPKSCDKTH |
| | TCPPCPAPELLGGPSVFLFPPKPKDTLMISRT |
| | PEVTCVVVDVSHEDPEVKFNWYVDGVEVHNAK |
| | TKPREEQYNSTYRVVSVLTVLHQDWLNGKEYK |
| | CKVSNKALPAPIEKTISKAKGQPREPQVYTLP |
| | PSREEMTKNQVSLTCLVKGFYPSDIAVEWESN |
| | GQPENNYKTTPPVLDSDGSFFLYSKLTVDKSR |
| | WQQGNVFSCSVMHEALHNHYTQKSLSLSPG |
| | Light Chain Variable Region (VL1.0) |
| 33 | syeLTQPRSVSGSPGQSVTISCTGTSSDVGGYNYV |
| | SWYQQHPGKAPKLMIFDVSKRPSGVPDRFSGS |
| | KSGNTASLTISGLOAEDEADYYCSSYADSWFG |
| | GGTKVTVL |
| | Heavy Chain Variable Region (VH1.0) |
| 34 | EVQLVQSGGGLVQPGGSLRLSCAASGFTFSSY |
| | AMSWVRQAPGKGLEWVSAISVSGGSTYYADSV |
| | KGRFTISRDNSKNTLYLQMNSLRAEDTAVYYC |
| | AKANWGFFDYWGQGTLVTVSS |
| | Light Chain Constant Region (CL) |
| 35 | GQPKAAPSVTLFPPSSEELQANKATLVCLISD |
| | FYPGAVTVAWKADSSPVKAGVETTTPSKQSNN |
| | KYAASSYLSLTPEQWKSHKSYSCQVTHEGSTV |
| | EKTVAPTECS |
| | Heavy Chain Constant Region (IgGlm3) |
| 36 | ASTKGPSVFPLAPSSKSTSGGTAALGCLVKDY |
| | FPEPVTVSWNSGALTSGVHTFPAVLQSSGLYS |
| | LSSVVTVPSSSLGTQTYICNVNHKPSNTKVDK |
| | RVEPKSCDKTHTCPPCPAPELLGGPSVFLFPP |
| | KPKDTLMISRTPEVTCVVVDVSHEDPEVKFNW |
| | YVDGVEVHNAKTKPREEQYNSTYRVVSVLTVL |
| | HQDWLNGKEYKCKVSNKALPAPIEKTISKAKG |
| | QPREPQVYTLPPSREEMTKNQVSLTCLVKGFY |
| | PSDIAVEWESNGQPENNYKTTPPVLDSDGSFF |
| | LYSKLTVDKSRWQQGNVFSCSVMHEALHNHYT |
| | QKSLSLSPG |
| | Antibody 3903E11 Hit: Nucleotide |
| | Light Chain Variable Region |
| 37 | TcctatgagCTGACACAGCCTCGCTCAGTGTC |
| | CGGGTCTCC |
| | TGGACAGTCAGTCACCATCTCCTGCACTGGAA |
| | CCAGCAGTGATGTTGGTGGTTATAACTATGTC |
| | TCCTGGTACCAACAGCACCCAGGCAAAGCCCC |
| | CAAACTCATGATTTTTGATGTCAGTAAGCGGC |
| | CCTCAGGGGTCCCTGATCGCTTCTCTGGCTCC |
| | AAGTCTGGCAACACGGCCTCCCTGACCATCTC |
| | TGGGCTCCAGGCTGAGGATGAGGCTGATTATT |
| | ACTGCTCCTCATATGCAGACAGCGTGGTATTC |
| | GGCGGAGGGACCAAGGTGACCGTCCTA |
| | Heavy Chain Variable Region |
| 38 | GAGGTGCAGCTGGTGCAGTCTGGGGGAGGCTT |
| | GGTACAGCCTGGGGGGTCCCTGAGACTCTCCT |
| | GTGCAGCCTCTGGATTCACCTTTAGCAGCTAT |
| | GCCATGAGCTGGGTCCGCCAGGCTCCAGGGAA |
| | GGGGCTGGAGTGGGTCTCAGCTATTAGTGTTA |
| | GTGGTGGTAGCACATACTACGCAGACTCCGTG |
| | AAGGGCCGATTCACCATCTCCAGAGACAATTC |
| | CAAGAACACGCTGTATCTGCAAATGAACAGCC |
| | TGAGAGCCGAGGACACGGCCGTATATTACTGT |
| | GCGAAAGCCAACTGGGGGTTCTTTGACTACTG |
| | GGGCCAGGGAACCCTGGTCACTGTCTCTTCA |
| | Light Chain Constant Region |
| 39 | GGACAGCCCAAGGCTGCCCCCTCGGTCACTCT |
| | GTTCCCGCCCTCCTCTGAGGAGCTTCAAGCCA |
| | ACAAGGCCACACTGGTGTGTCTCATAAGTGAC |
| | TTCTACCCGGGAGCCGTGACAGTGGCCTGG |
| | AAGGCAGATAGCAGCCCCGTCAAGGCGGGAG |
| | TGGAGACCACCACACCCTCCAAACAAAGCAA |
| | CAACAAGTACGCGGCCAGCAGCTACCTGAGC |
| | CTGACGCCTGAGCAGTGGAAGTCCCACAAAA |
| | GCTACAGCTGCCAGGTCACGCATGAAGGGAG |
| | CACCGTGGAGAAGACAGTGGCCCCTACAGAA |
| | TGTTCA |
| | Heavy Chain Constant Region |
| | (IgGlm3) |
| 40 | GCTAGCACCAAGGGCCCATCGGTCTTCCCCC |
| | TGGCACCCTCCTCCAAGAGCACCTCTGGGGG |
| | CACAGCGGCCCTGGGCTGCCTGGTCAAGGAC |
| | TACTTCCCCGAACCGGTGACGGTGTCGTGGA |
| | ACTCAGGCGCCCTGACCAGCGGCGTGCACAC |
| | CTTCCCGGCTGTCCTACAGTCCTCAGGACTC |
| | TACTCCCTCAGCAGCGTGGTGACCGTGCCCT |
| | CCAGCAGCTTGGGCACCCAGACCTACATCTG |
| | CAACGTGAATCACAAGCCCAGCAACACCAAG |
| | GTGGACAAGAGAGTTGAGCCCAAATCTTGTG |
| | ACAAAACTCACACATGCCCACCGTGCCCAGC |
| | ACCTGAACTCCTGGGGGGACCGTCAGTCTTC |
| | CTCTTCCCCCCAAAACCCAAGGACACCCTCA |
| | TGATCTCCCGGACCCCTGAGGTCACATGCGT |
| | GGTGGTGGACGTGAGCCACGAAGACCCTGAG |
| | GTCAAGTTCAACTGGTACGTGGACGGCGTGG |
| | AGGTGCATAATGCCAAGACAAAGCCGCGGGA |
| | GGAGCAGTACAACAGCACGTACCGTGTGGTC |
| | AGCGTCCTCACCGTCCTGCACCAGGACTGGC |
| | TGAATGGCAAGGAGTACAAGTGCAAGGTCTC |
| | CAACAAAGCCCTCCCAGCCCCCATCGAGAAA |
| | ACCATCTCCAAAGCCAAAGGGCAGCCCCGAG |
| | AACCACAGGTGTACACCCTGCCCCCATCACG |
| | GGAGGAGATGACCAAGAACCAGGTCAGCCTG |
| | ACCTGCCTGGTCAAAGGCTTCTATCCCAGCG |
| | ACATCGCCGTGGAGTGGGAGAGCAATGGGCA |
| | GCCGGAGAACAACTACAAGACCACGCCTCCC |
| | GTGCTGGACTCCGACGGCTCCTTCTTCCTCT |
| | ATAGCAAGCTCACCGTGGACAAGAGCAGGTG |
| | GCAGCAGGGGAACGTCTTCTCATGCTCCGTG |
| | ATGCATGAGGCTCTGCACAACCACTACACGC |
| | AGAAGAGCCTCTCCCTGTCCCCGGGT |

TIM3 Sequences (and others)

SEQ ID NO: 41: human TIM-3 extracellular
domain (amino acid sequence,
NP_116171)
SEVEYRAEVGQNAYLPCFYTPAAPGNLVPVC
WGKGACPVFECGNVVLRTDERDVNYWTSRYW
LNGDFRKGDVSLTIENVTLADSGIYCCRIQI
PGIMNDEKFNLKLVIKPAKVTPAPTRQRDFT
AAFPRMLTTRGHGPAETQTLGSLPDINLTQI
STLANELRDSRLANDLRDSGATIRIG
SEQ ID NO: 42: cyno TIM-3 extracellular
domain (amino acid sequence,
XP_005558438)
SEVEYIAEVGQNAYLPCSYTPAPPGNLVPVC
WGKGACPVFDCSNWLRTDNRDVNDRTSGRYW
LKGDFHKGDVSLTIENVTLADSGVYCCRIQI
PGIMNDEKHNVKLWIKPAKVTPAPTLQRDLT
SAFPRMLTTGEHGPAETQTPGSLPDVNLTQI
FTLTNELRDSGATIRTA
SEQ ID NO: 43: human TIM-3 ECD
with His tag (amino acid
sequence, Novoprotein
Cat# C356)
SEVEYRAEVGQNAYLPCFYTPAAPGNLVPVC
WGKGACPVFECGNVVLRTDERDVNYWTSRYW
LNGDFRKGDVSLTIENVTLADSGIYCCRIQI
PGIMNDEKFNLKLVIKPAKVTPAPTLQRDFT
AAFPRMLTTRGHGPAETQTLGSLPDINLTQI
STLANELRDSRLANDLRDSGATIRVDHHHHH
H
SEQ ID NO: 44: human TIM-3 ECD
with His tag (amino acid
sequence, Novoprotein
Cat# CD71)
SEVEYRAEVGQNAYLPCFYTPAAPGNLVPVC
WGKGACPVFECGNVVLRTDERDVNYWTSRYW
LNGDFRKGDVSLTIENVTLADSGIYCCRIQI
PGIMNDEKFNLKLVIKPAKVTPAPTLQRDFT
AAFPRMLTTRGHGPAETQTLGSLPDINLTQI
STLANELRDSRLANDLRDSGATIRVDDIEGR
MDEPKSCDKTHTCPPCPAPELLGGPSVFLFP
PKPKDTLMISRTPEVTCVVVDVSHEDPEVKF
NWYVDGVEVHNAKTKPREEQYNSTYRVVSVL
TVLHQDWLNGKEYKCKVSNKALPAPIEKTIS
KAKGQPREPQVYTLPPSREEMTKNQVSLTCL
VKGFYPSDIAVEWESNGQPENNYKTTPPVLD
SDGSFFLYSKLTVDKSRWQQGNVFSCSVMHE
ALHNHYTQKSLSLSPGKHHHHHH
SEQ ID NO: 45: marmoset TIM-3 ECD
(amino acid
sequence, Novoprotein
Cat# CM64)
EEYIVEVGQNAYLPCFYTLDTPGNLVPVCWG
KGACPVFECGDVVLRTDERDVSYRTSSRYWL
NGDFHKGNVTLAIGNVTLEDSGIYCCRVQIP
GIMNDKKFNLKLVIKPAKVTPAPTLPRDSTP
AFPRMLTTEDHGPAETQTLEILHDKNLTQLS
TLANELQDAGTTIRIHHHHHH
SEQ ID NO: 46: mouse TIM-3
extracellular domain (amino
acid sequence,
NP_599011)
RSLENAYVFEVGKNAYLPCSYTLSTPGALVP
MCWGKGFCPWSQCTNELLRTDERNVTYQKSS
RYQLKGDLNKGDVSLIIKNVTLDDHGTYCCR
IQFPGLMNDKKLELKLDIKAAKVTPAQTAHG
DSTTASPRTLTTERNGSETQTLVTLHNNNGT
KISTWADEIKDSGETIRTA
SEQ ID NO: 47: HC of 3903E11 Fab
fragment for crystallization
EVQLVESGGGLVQPGGSLRLSCAASGFTFSS
YAMSWVRQAPGKGLEWVSAISVSGGSTYYAD
SVKGRFTISRDNSKNTLYLQMNSLRAEDTAV
YYCAKANWGFFDYWGQGTLVTVSSASTKGPS
VFPLAPSSKSTSGGTAALGCLVKDYFPEPVT
VSWNSGALTSGVHTFPAVLQSSGLYSLSSWT
VPSSSLGTQTYICNVNHKPSNTKVDKKVEPK
SCAAAHHHHHH
SEQ ID NO: 48: LC of 3903E11 Fab
fragment for crystallization
SYELTQPRSVSGSPGQSVTISCTGTSSDVGG
YNYVSWYQQHPGKAPKLMIFDVSKRPSGVPD
RFSGSKSGNTASLTISGLQAEDEADYYCSSY
ADSVVFGGGTKVTVLGQPKAAPSVTLFPPSS
EELQANKATLVCLISDFYPGAVTVAWKADSS
PVKAGVETTTPSKQSNNKYAASSYLSLTPEQ
WKSHKSYSCQVTHEGSTVEKTVAPTECS
SEQ ID NO: 49: Human TIM-3 ECD
(expressed, in e. coli for
crystallography)
MSEVEYRAEVGQNAYLPCFYTPAAPGNLVPV
CWGKGACPVFECGNVVLRTDERDVNYWTSRY
WLNGDFRKGDVSLTIENVTLADSGIYCCRIQ
IPGIMNDEKFNLKLVIK
SEQ ID NO: 50: Nucleotide
sequence
for Human TIM-3 ECD (expressed
in e. coli for crystallography)
ATGAGCGAGGTGGAATATCGGGCCGAAGTGG
GCCAGAACGCCTACCTGCCTTGCTTCTACAC
ACCAGCCGCCCCTGGCAACCTGGTGCCTGTG
TGTTGGGGAAAGGGCGCCTGCCCTGTGTTCG
AGTGCGGCAACGTGGTGCTGAGAACCGACGA
GCGGGACGTGAACTACTGGACCAGCCGGTAC
TGGCTGAACGGCGACTTCAGAAAGGGCGACG
TGTCCCTGACCATCGAGAACGTGACCCTGGC
CGACAGCGGCATCTACTGCTGCAGAATCCAG
ATCCCCGGCATCATGAACGACGAGAAGTTCA
ACCTGAAGCTCGTGATCAAGTAA
SEQ ID NO: 51 Human TIM-3 Isoform 1
(Uniprot Code Q8TDQ0-1)
MFSHLPFDCVLLLLLLLLTRSSEVEYRAEVG
QNAYLPCFYTPAAPGNLVPVCWGKGACPVFE
CGNVVLRTDERDVNYWTSRYWLNGDFRKGDV
SLTIENVTLADSGIYCCRIQIPGIMNDEKFN
LKLVIKPAKVTPAPTRQRDFTAAFPRMLTTR
GHGPAETQTLGSLPDINLTQISTLANELRDS
RLANDLRDSGATIRIGIYIGAGICAGLALAL
IFGALIFKWYSHSKEKIQNLSLISLANLPPS
GLANAVAEGIRSEENIYTIEENVYEVEEPNE
YYCYVSSRQQPSQPLGCRFAMP

1. A method of treating cancer in a mammal, the method comprising administering an effective amount of an anti-TIM-3 antibody and a second therapeutic agent to the mammal in need thereof.

2. The method of claim 1, wherein the anti-TIM-3 antibody is administered in an amount of from about 0.1 mg/kg to about 100 mg/kg.

3. The method of claim 1, wherein the anti-TIM-3 antibody is administered as a flat (fixed) dose of from about 5 mg to about 3500 mg.

4. The method of claim 1, wherein the second therapeutic agent is an anti-PD-L1/TGFβ Trap fusion protein.

5. The method of claim 4, wherein the anti-PD-L1/TGFβ Trap fusion protein comprises:
(a) a heavy chain comprising an CDR_{H1}, an CDR_{H2}, and an CDR_{H3}, having at least 80% overall sequence identity to SYIMM (SEQ ID NO: 78), SIYPSGGITFYADTVKG (SEQ ID NO: 79), and IKLGTVTTVDY (SEQ ID NO: 80), respectively, and
(b) a light chain comprising an CDR_{L1}, an CDR_{L2}, and an CDR_{L3}, having at least 80% overall sequence identity to TGTSSDVGGYNYVS (SEQ ID NO: 81), DVSNRPS (SEQ ID NO: 82), and SSYTSSSTRV (SEQ ID NO: 83), respectively.

6. The method of claim 4, wherein the anti-PD-L1/TGFβ Trap fusion protein is a protein having the amino acid sequence of bintrafusp alfa.

7. The method of claim 6, wherein the protein is bintrafusp alfa.

8. The method of claim 4, wherein the anti-PD-L1/TGFβ Trap fusion protein is administered in a flat (fixed) dose of from about 800 mg to about 2600 mg.

9. The method of claim 8, wherein the anti-PD-L1/TGFβ Trap fusion protein is administered in a flat (fixed) dose of about 1200 mg.

10. The method of claim 8, wherein the anti-PD-L1/TGFβ Trap fusion protein is administered in a flat (fixed) dose of about 2400 mg.

11. The method of claim 1, wherein the anti-TIM-3 antibody and/or the anti-PD-L1/TGFβ Trap fusion protein is administered every two weeks.

12. The method of claim 1, wherein the anti-TIM-3 antibody and/or the anti-PD-L1/TGFβ Trap fusion protein is administered every three weeks.

13. The method of claim 1, wherein the cancer is selected from the group consisting of diffuse large B-cell lymphoma, renal cell carcinoma (RCC), non-small cell lung carcinoma (NSCLC), squamous cell carcinoma of the head and neck (SCCHN), triple negative breast cancer (TNBC) or gastric/stomach adenocarcinoma (STAD).

14. The method of claim 1, wherein the mammal is a human.

15. The method of claim 1 wherein the anti-TIM-3 antibody comprises
(i) an immunoglobulin heavy chain variable region comprising a CDR_{H1} comprising the amino acid sequence of SEQ ID NO: 1, a CDR_{H2} comprising the amino acid sequence of SEQ ID NO: 2, and a CDR_{H3} comprising the amino acid sequence of SEQ ID NO: 3; and
(ii) an immunoglobulin light chain variable region comprising a CDR_{L1} comprising the amino acid sequence of SEQ ID NO: 4, a CDR_{L2} comprising the amino acid sequence of SEQ ID NO: 5, and a CDR_{L3} comprising the amino acid sequence of SEQ ID NO: 6.

16. The method of claim 1 wherein the anti-TIM-3 antibody comprises an immunoglobulin heavy chain variable region selected from the group consisting of SEQ ID NO: 53, SEQ ID NO: 24, SEQ ID NO: 55, SEQ ID NO: 34, and an immunoglobulin light chain variable region selected from the group consisting of SEQ ID NO: 52, SEQ ID NO: 54, SEQ ID NO: 23 and SEQ ID NO: 33.

17. The method of claim 1 wherein the anti-TIM-3 antibody comprises an immunoglobulin heavy chain variable region comprising the amino acid sequence of SEQ ID NO: 24, and an immunoglobulin light chain variable region comprising the amino acid sequence of SEQ ID NO: 23.

18. The method of claim 1 wherein the anti-TIM-3 antibody comprises an immunoglobulin heavy chain and an immunoglobulin light chain selected from the group consisting of:
(a) an immunoglobulin heavy chain comprising the amino acid sequence of SEQ ID NO: 22, and an immunoglobulin light chain comprising the amino acid sequence of SEQ ID NO: 21; and
(b) an immunoglobulin heavy chain comprising the amino acid sequence of SEQ ID NO: 32, and an immunoglobulin light chain comprising the amino acid sequence of SEQ ID NO: 31.

19. The method of claim 1 wherein the anti-TIM-3 antibody has a KD of 9.2 nM or lower, as measured by surface plasmon resonance.

20. The method of claim 1, wherein the anti-TIM-3 antibody competes with a second anti-TIM-3 antibody for binding to the galectin-9 binding site on human TIM-3, wherein the second anti-TIM-3 antibody comprises
(i) an immunoglobulin heavy chain variable region comprising a CDR_{H1} comprising the amino acid sequence of SEQ ID NO: 1, a CDR_{H2} comprising the amino acid sequence of SEQ ID NO: 2, and a CDR_{H3} comprising the amino acid sequence of SEQ ID NO: 3; and
(ii) an immunoglobulin light chain variable region comprising a CDR_{L1} comprising the amino acid sequence of SEQ ID NO: 4, a CDR_{L2} comprising the amino acid sequence of SEQ ID NO: 5, and a CDR_{L3} comprising the amino acid sequence of SEQ ID NO: 6.

21. The method of claim 1, wherein the anti-TIM-3 antibody competes with a second anti-TIM-3 antibody for binding to the PtdSer binding site on human TIM-3, wherein the second anti-TIM-3 antibody comprises
(i) an immunoglobulin heavy chain variable region comprising a CDR_{H1} comprising the amino acid sequence of SEQ ID NO: 1, a CDR_{H2} comprising the amino acid sequence of SEQ ID NO: 2, and a CDR_{H3} comprising the amino acid sequence of SEQ ID NO: 3; and
(ii) an immunoglobulin light chain variable region comprising a CDR_{L1} comprising the amino acid sequence of SEQ ID NO: 4, a CDR_{L2} comprising the amino acid sequence of SEQ ID NO: 5, and a CDR_{L3} comprising the amino acid sequence of SEQ ID NO: 6.

22. The method of claim 1, wherein the anti-TIM-3 antibody competes with a second anti-TIM-3 antibody for binding to the carcinoembryonic antigen cell adhesion-related molecule 1 (CEACAM1) binding site on human TIM-3, wherein the second anti-TIM-3 antibody comprises
(i) an immunoglobulin heavy chain variable region comprising a CDR_{H1} comprising the amino acid sequence of SEQ ID NO: 1, a CDR_{H2} comprising the amino acid sequence of SEQ ID NO: 2, and a CDR_{H3} comprising the amino acid sequence of SEQ ID NO: 3; and
(ii) an immunoglobulin light chain variable region comprising a CDR_{L1} comprising the amino acid sequence of SEQ ID NO: 4, a CDR_{L2} comprising the amino acid sequence of SEQ ID NO: 5, and a CDR_{L3} comprising the amino acid sequence of SEQ ID NO: 6.

23. The method of claim 1, wherein the anti-TIM-3 antibody is an antibody comprising an epitope comprising P59, F61 and E62 of the human TIM-3 protein.

24.-61. (canceled)


SEQUENCE LISTING (118 sequences)

SEQ ID NO: 1 - protein, 8 aa, Artificial Sequence
  source: /note=“Description of Artificial Sequence: Synthetic peptide”
GFTFSSYA

SEQ ID NO: 2 - protein, 8 aa, Artificial Sequence
  source: /note=“Description of Artificial Sequence: Synthetic peptide”
ISVSGGST

SEQ ID NO: 3 - protein, 10 aa, Artificial Sequence
  source: /note=“Description of Artificial Sequence: Synthetic peptide”
AKANWGFFDY

SEQ ID NO: 4 - protein, 9 aa, Artificial Sequence
  source: /note=“Description of Artificial Sequence: Synthetic peptide”
SSDVGGYNY

SEQ ID NO: 5 - protein, 3 aa, Artificial Sequence
  source: /note=“Description of Artificial Sequence: Synthetic peptide”
DVS

SEQ ID NO: 6 - protein, 8 aa, Artificial Sequence
  source: /note=“Description of Artificial Sequence: Synthetic peptide”
SSYADSVV

SEQ ID NO: 7 - protein, 25 aa, Artificial Sequence
  source: /note=“Description of Artificial Sequence: Synthetic peptide”
  VARIANT (6)..(6): /replace=“Glu”
  SITE (1)..(25): /note=“Variant residues given in the sequence have no preference with respect to those in the annotations for variant positions”
EVQLVQSGGGLVQPGGSLRLSCAAS

SEQ ID NO: 8 - protein, 17 aa, Artificial Sequence
  source: /note=“Description of Artificial Sequence: Synthetic peptide”
  VARIANT (1)..(1): /replace=“Leu”
  SITE (1)..(17): /note=“Variant residues given in the sequence have no preference with respect to those in the annotations for variant positions”
MSWVRQAPGKGLEWVSA

SEQ ID NO: 9 - protein, 38 aa, Artificial Sequence
  source: /note=“Description of Artificial Sequence: Synthetic polypeptide”
YYADSVKGRFTISRDNSKNTLYLQMNSLRAEDTAVYYC

SEQ ID NO: 10 - protein, 11 aa, Artificial Sequence
  source: /note=“Description of Artificial Sequence: Synthetic peptide”
WGQGTLVTVSS

SEQ ID NO: 11 - protein, 25 aa, Artificial Sequence
  source: /note=“Description of Artificial Sequence: Synthetic peptide”
  VARIANT (1)..(1): /replace=“Gln”
  VARIANT (2)..(2): /replace=“Ser”
  VARIANT (3)..(3): /replace=“Ala”
  SITE (1)..(25): /note=“Variant residues given in the sequence have no preference with respect to those in the annotations for variant positions”
SYELTQPRSVSGSPGQSVTISCTGT

SEQ ID NO: 12 - protein, 17 aa, Artificial Sequence
  source: /note=“Description of Artificial Sequence: Synthetic peptide”
  VARIANT (17)..(17): /replace=“Tyr”
  SITE (1)..(17): /note=“Variant residues given in the sequence have no preference with respect to those in the annotations for variant positions”
VSWYQQHPGKAPKLMIF

SEQ ID NO: 13 - protein, 36 aa, Artificial Sequence
  source: /note=“Description of Artificial Sequence: Synthetic polypeptide”
KRPSGVPDRFSGSKSGNTASLTISGLQAEDEADYYC

SEQ ID NO: 14 - protein, 10 aa, Artificial Sequence
  source: /note=“Description of Artificial Sequence: Synthetic peptide”
FGGGTKVTVL

SEQ ID NO: 15 - protein, 214 aa, Artificial Sequence
  source: /note=“Description of Artificial Sequence: Synthetic polypeptide”
QSALTQPRSVSGSPGQSVTISCTGTSSDVGGYNYVSWYQQHPGKAPKLMIFDVSKRPSGVPDRFSGSKSGNTASLTISGLQAEDEADYYCSSYADSVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 16 - protein, 446 aa, Artificial Sequence
  source: /note=“Description of Artificial Sequence: Synthetic polypeptide”
EVQLVQSGGGLVQPGGSLRLSCAASGFTFSSYALSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 214 aa, Artificial Sequence
  source: /note=“Description of Artificial Sequence: Synthetic polypeptide”
SYELTQPRSVSGSPGQSVTISCTGTSSDVGGYNYVSWYQQHPGKAPKLMIYDVSKRPSGVPDRFSGSKSGNTASLTISGLQAEDEADYYCSSYADSVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 18 - protein, 446 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 214 aa, Artificial Sequence; identical to SEQ ID NO: 15
  source: /note=“Description of Artificial Sequence: Synthetic polypeptide”

SEQ ID NO: 20 - protein, 446 aa, Artificial Sequence
  source: /note=“Description of Artificial Sequence: Synthetic polypeptide”
EVQLVESGGGLVQPGGSLRLSCAASGFTFSSYALSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 214 aa, Artificial Sequence
  source: /note=“Description of Artificial Sequence: Synthetic polypeptide”
QSALTQPRSVSGSPGQSVTISCTGTSSDVGGYNYVSWYQQHPGKAPKLMIYDVSKRPSGVPDRFSGSKSGNTASLTISGLQAEDEADYYCSSYADSVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 22 - protein, 445 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSSDKTHTCPPCPAPPVAGPSVFLFPPKPKDTLMISRTPEVTCVVVDVSHEDPEVQFNWYVDGVEVHNAKTKPREEQAQSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 23 - protein, 108 aa, Artificial Sequence
  source: /note=“Description of Artificial Sequence: Synthetic polypeptide”
QSALTQPRSVSGSPGQSVTISCTGTSSDVGGYNYVSWYQQHPGKAPKLMIYDVSKRPSGVPDRFSGSKSGNTASLTISGLQAEDEADYYCSSYADSVVFGGGTKVTVL

SEQ ID NO: 24 - protein, 117 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSS

SEQ ID NO: 25 - protein, 106 aa, Artificial Sequence
  source: /note=“Description of Artificial Sequence: Synthetic polypeptide”
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 26 - protein, 328 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSSDKTHTCPPCPAPPVAGPSVFLFPPKPKDTLMISRTPEVTCVVVDVSHEDPEVQFNWYVDGVEVHNAKTKPREEQAQSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 27 - DNA, 326 bp, Artificial Sequence
  source: /note=“Description of Artificial Sequence: Synthetic polynucleotide”
cagagcgccctgacacagcctcgctcagtgtccgggtctcctggacagtcagtcaccatctcctgcactggaaccagcagtgatgttggtggttataactatgtctcctggtaccaacagcacccaggcaaagcccccaaactcatgatttacgatgtcagtaagcggccctcaggggtccctgatcgcttctctggctccaagtctggcaacacggcctccctgaccatctctgggctccaggctgaggatgaggctgattattactgctcctcatatgcagacagcgtggtattcggcggagggaccaaggtgaccgtcctagg

SEQ ID NO: 28 - DNA, 351 bp, Artificial Sequence
  source: /note=“Description of Artificial Sequence: Synthetic polynucleotide”
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtgttagtggtggtagcacatactacgcagactccgtgaagggccgattcaccatctccagagacaattccaagaacacgctgtatctgcaaatgaacagcctgagagccgaggacacggccgtatattactgtgcgaaagccaactgggggttctttgactactggggccagggaaccctggtcactgtctcttca

SEQ ID NO: 29 - DNA, 318 bp, Artificial Sequence
  source: /note=“Description of Artificial Sequence: Synthetic polynucleotide”
ggacagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 30 - DNA, 984 bp, Artificial Sequence
  source: /note=“Description of Artificial Sequence: Synthetic polynucleotide”
gctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttctgacaaaactcacacatgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcaggcccagagcacgttccgtgtggtcagcgtcctcaccgttgtgcaccaggactggctgaacggcaaggagtacaagtgcgctgtctccaacaaaggcctcccagcccccatcgagaaaaccatctccaaaaccaaagggcagccccgagaaccacaggtgtacaccctgcccccatcacgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtccccgggt

SEQ ID NO: 31 - protein, 214 aa, Artificial Sequence
  source: /note=“Description of Artificial Sequence: Synthetic polypeptide”
SYELTQPRSVSGSPGQSVTISCTGTSSDVGGYNYVSWYQQHPGKAPKLMIFDVSKRPSGVPDRFSGSKSGNTASLTISGLQAEDEADYYCSSYADSVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 32 - protein, 446 aa, Artificial Sequence
  source: /note=“Description of Artificial Sequence: Synthetic polypeptide”
EVQLVQSGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 108 aa, Artificial Sequence
  source: /note=“Description of Artificial Sequence: Synthetic polypeptide”
SYELTQPRSVSGSPGQSVTISCTGTSSDVGGYNYVSWYQQHPGKAPKLMIFDVSKRPSGVPDRFSGSKSGNTASLTISGLQAEDEADYYCSSYADSVVFGGGTKVTVL

SEQ ID NO: 34 - protein, 117 aa, Artificial Sequence
  source: /note=“Description of Artificial Sequence: Synthetic polypeptide”
EVQLVQSGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSS

SEQ ID NO: 35 - protein, 106 aa, Artificial Sequence; identical to SEQ ID NO: 25
  source: /note=“Description of Artificial Sequence: Synthetic polypeptide”

SEQ ID NO: 36 - protein, 329 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - DNA, 324 bp, Artificial Sequence
  source: /note=“Description of Artificial Sequence: Synthetic polynucleotide”
tcctatgagctgacacagcctcgctcagtgtccgggtctcctggacagtcagtcaccatctcctgcactggaaccagcagtgatgttggtggttataactatgtctcctggtaccaacagcacccaggcaaagcccccaaactcatgatttttgatgtcagtaagcggccctcaggggtccctgatcgcttctctggctccaagtctggcaacacggcctccctgaccatctctgggctccaggctgaggatgaggctgattattactgctcctcatatgcagacagcgtggtattcggcggagggaccaaggtgaccgtccta

SEQ ID NO: 38 - DNA, 351 bp, Artificial Sequence
  source: /note=“Description of Artificial Sequence: Synthetic polynucleotide”
gaggtgcagctggtgcagtctgggggaggcttggtacagcctggggggtccctgagactctcctgtgcagcctctggattcacctttagcagctatgccatgagctgggtccgccaggctccagggaaggggctggagtgggtctcagctattagtgttagtggtggtagcacatactacgcagactccgtgaagggccgattcaccatctccagagacaattccaagaacacgctgtatctgcaaatgaacagcctgagagccgaggacacggccgtatattactgtgcgaaagccaactgggggttctttgactactggggccagggaaccctggtcactgtctcttca

SEQ ID NO: 39 - DNA, 318 bp, Artificial Sequence; identical to SEQ ID NO: 29
  source: /note=“Description of Artificial Sequence: Synthetic polynucleotide”

SEQ ID NO: 40 - DNA, 987 bp, Artificial Sequence
  source: /note=“Description of Artificial Sequence: Synthetic polynucleotide”
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 41 - protein, 181 aa, Homo sapiens
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 42 - protein, 174 aa, Macaca fascicularis
SEVEYIAEVGQNAYLPCSYTPAPPGNLVPVCWGKGACPVFDCSNVVLRTDNRDVNDRTSGRYWLKGDFHKGDVSLTIENVTLADSGVYCCRIQIPGIMNDEKHNVKLVVIKPAKVTPAPTLQRDLTSAFPRMLTTGEHGPAETQTPGSLPDVNLTQIFTLTNELRDSGATIRTA

SEQ ID NO: 43 - protein, 187 aa, Artificial Sequence
  source: /note=“Description of Artificial Sequence: Synthetic polypeptide”
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRVDHHHHHH

SEQ ID NO: 44 - protein, 426 aa, Artificial Sequence
  source: /note=“Description of Artificial Sequence: Synthetic polypeptide”
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRVDDIEGRMDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 45 - protein, 176 aa, Artificial Sequence
  source: /note=“Description of Artificial Sequence: Synthetic polypeptide”
EEYIVEVGQNAYLPCFYTLDTPGNLVPVCWGKGACPVFECGDVVLRTDERDVSYRTSSRYWLNGDFHKGNVTLAIGNVTLEDSGIYCCRVQIPGIMNDKKFNLKLVIKPAKVTPAPTLPRDSTPAFPRMLTTEDHGPAETQTLEILHDKNLTQLSTLANELQDAGTTIRIHHHHHH

SEQ ID NO: 46 - protein, 174 aa, Mus musculus
RSLENAYVFEVGKNAYLPCSYTLSTPGALVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKDSGETIRTA

SEQ ID NO: 47 - protein, 229 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHH

SEQ ID NO: 48 - protein, 214 aa, Artificial Sequence; identical to SEQ ID NO: 31
  source: /note=“Description of Artificial Sequence: Synthetic polypeptide”

SEQ ID NO: 49 - protein, 110 aa, Artificial Sequence
  source: /note=“Description of Artificial Sequence: Synthetic polypeptide”
MSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIK

SEQ ID NO: 50 - DNA, 333 bp, Artificial Sequence
  source: /note=“Description of Artificial Sequence: Synthetic polynucleotide”
atgagcgaggtggaatatcgggccgaagtgggccagaacgcctacctgccttgcttctacacaccagccgcccctggcaacctggtgcctgtgtgttggggaaagggcgcctgccctgtgttcgagtgcggcaacgtggtgctgagaaccgacgagcgggacgtgaactactggaccagccggtactggctgaacggcgacttcagaaagggcgacgtgtccctgaccatcgagaacgtgaccctggccgacagcggcatctactgctgcagaatccagatccccggcatcatgaacgacgagaagttcaacctgaagctcgtgatcaagtaa

SEQ ID NO: 51 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 52 - protein, 108 aa, Artificial Sequence
  source: /note=“Description of Artificial Sequence: Synthetic polypeptide”
QSALTQPRSVSGSPGQSVTISCTGTSSDVGGYNYVSWYQQHPGKAPKLMIFDVSKRPSGVPDRFSGSKSGNTASLTISGLQAEDEADYYCSSYADSVVFGGGTKVTVL

SEQ ID NO: 53 - protein, 117 aa, Artificial Sequence
  source: /note=“Description of Artificial Sequence: Synthetic polypeptide”
EVQLVQSGGGLVQPGGSLRLSCAASGFTFSSYALSWVRQAPGKGLEWVSAISVSGGSTYYADSVKGRFTISRDNSKNTLYLQMNSLRAEDTAVYYCAKANWGFFDYWGQGTLVTVSS

SEQ ID NO: 54 - protein, 108 aa, Artificial Sequence
  source: /note=“Description of Artificial Sequence: Synthetic polypeptide”
SYELTQPRSVSGSPGQSVTISCTGTSSDVGGYNYVSWYQQHPGKAPKLMIYDVSKRPSGVPDRFSGSKSGNTASLTISGLQAEDEADYYCSSYADSVVFGGGTKVTVL

SEQ ID NO: 55 - protein, 117 aa, Artificial Sequence
  source: /note=“Description of Artificial Sequence: Synthetic polypeptide”
EVQLVESGGGLVQPGGSLRLSCAASGFTFSSYALSWVRQAPGKGLEWVSAISVSGGSTYYADSVKGRFTISRDNSKNTLYLQMNSLRAEDTAVYYCAKANWGFFDYWGQGTLVTVSS

SEQ ID NO: 56-57 - skipped (2 entries)

SEQ ID NO: 58 - protein, 5 aa, Artificial Sequence
  source: /note=“Description of Artificial Sequence: Synthetic peptide”
  VARIANT (1)..(1): /replace=“Arg” or “Thr” or “Gln” or “Gly” or “Ala” or “Trp” or “Met” or “Ile” or “Ser”
  VARIANT (3)..(3): /replace=“Arg” or “Lys” or “Leu” or “Met” or “Ile”
  VARIANT (5)..(5): /replace=“Thr” or “Asn” or “Gln” or “Ala” or “Val” or “Tyr” or “Trp” or “Phe” or “Met”
  SITE (1)..(5): /note=“Variant residues given in the sequence have no preference with respect to those in the annotations for variant positions”
  source: /note=“See specification as filed for detailed description of substitutions and preferred embodiments”
KYVMH

SEQ ID NO: 59 - protein, 17 aa, Artificial Sequence
  source: /note=“Description of Artificial Sequence: Synthetic peptide”
  VARIANT (8)..(8): /replace=“Ile”
  VARIANT (14)..(14): /replace=“Thr”
  SITE (1)..(17): /note=“Variant residues given in the sequence have no preference with respect to those in the annotations for variant positions”
  source: /note=“See specification as filed for detailed description of substitutions and preferred embodiments”
SIYPSGGFTFYADSVKG

SEQ ID NO: 60 - protein, 11 aa, Artificial Sequence
  source: /note=“Description of Artificial Sequence: Synthetic peptide”
  VARIANT (10)..(10): /replace=“Asp”
  SITE (1)..(11): /note=“Variant residues given in the sequence have no preference with respect to those in the annotations for variant positions”
  source: /note=“See specification as filed for detailed description of substitutions and preferred embodiments”
IKLGTVTTVEY

SEQ ID NO: 61 - protein, 30 aa, Artificial Sequence
  source: /note=“Description of Artificial Sequence: Synthetic polypeptide”
EVQLLESGGGLVQPGGSLRLSCAASGFTFS

SEQ ID NO: 62 - protein, 14 aa, Artificial Sequence
  source: /note=“Description of Artificial Sequence: Synthetic peptide”
WVRQAPGKGLEWVS

SEQ ID NO: 63 - protein, 32 aa, Artificial Sequence
  source: /note=“Description of Artificial Sequence: Synthetic polypeptide”
RFTISRDNSKNTLYLQMNSLRAEDTAVYYCAR

SEQ ID NO: 64 - protein, 11 aa, Artificial Sequence
  source: /note=“Description of Artificial Sequence: Synthetic peptide”
WGQGTLVTVSS

SEQ ID NO: 65 - protein, 14 aa, Artificial Sequence
  source: /note=“Description of Artificial Sequence: Synthetic peptide”
  VARIANT (4)..(4): /replace=“Ser”
  VARIANT (5)..(5): /replace=“Arg” or “Ser”
  VARIANT (9)..(9): /replace=“Gly”
  SITE (1)..(14): /note=“Variant residues given in the sequence have no preference with respect to those in the annotations for variant positions”
  source: /note=“See specification as filed for detailed description of substitutions and preferred embodiments”
TGTNTDVGAYNYVS

SEQ ID NO: 66 - protein, 7 aa, Artificial Sequence
  source: /note=“Description of Artificial Sequence: Synthetic peptide”
  VARIANT (1)..(1): /replace=“Asp”
  VARIANT (3)..(3): /replace=“Asn” or “Ser”
  VARIANT (4)..(4): /replace=“His” or “Asn”
  SITE (1)..(7): /note=“Variant residues given in the sequence have no preference with respect to those in the annotations for variant positions”
  source: /note=“See specification as filed for detailed description of substitutions and preferred embodiments”
EVIDRPS

SEQ ID NO: 67 - protein, 10 aa, Artificial Sequence
  source: /note=“Description of Artificial Sequence: Synthetic peptide”
  VARIANT (3)..(3): /replace=“Tyr”
  VARIANT (5)..(5): /replace=“Ser”
  VARIANT (6)..(6): /replace=“Thr” or “Ser”
  VARIANT (7)..(7): /replace=“Ser”
  VARIANT (8)..(8): /replace=“Thr”
  SITE (1)..(10): /note=“Variant residues given in the sequence have no preference with respect to those in the annotations for variant positions”
  source: /note=“See specification as filed for detailed description of substitutions and preferred embodiments”
SSFTNRGIRV

SEQ ID NO: 68 - protein, 22 aa, Artificial Sequence
  source: /note=“Description of Artificial Sequence: Synthetic peptide”
QSALTQPASVSGSPGQSITISC

SEQ ID NO: 69 - protein, 15 aa, Artificial Sequence
  source: /note=“Description of Artificial Sequence: Synthetic peptide”
WYQQHPGKAPKLMIY

SEQ ID NO: 70 - protein, 32 aa, Artificial Sequence
  source: /note=“Description of Artificial Sequence: Synthetic polypeptide”
GVSNRFSGSKSGNTASLTISGLQAEDEADYYC

SEQ ID NO: 71 - protein, 10 aa, Artificial Sequence
  source: /note=“Description of Artificial Sequence: Synthetic peptide”
FGTGTKVTVL

SEQ ID NO: 72 - protein, 5 aa, Artificial Sequence
  source: /note=“Description of Artificial Sequence: Synthetic peptide”
  VARIANT (1)..(1): /replace=“Arg” or “Thr” or “Gln” or “Gly” or “Ala” or “Trp” or “Met” or “Ile” or “Ser”
  VARIANT (3)..(3): /replace=“Arg” or “Lys” or “Leu” or “Met” or “Ile”
  VARIANT (5)..(5): /replace=“Thr” or “Asn” or “Gln” or “Ala” or “Val” or “Tyr” or “Trp” or “Phe” or “Met”
  SITE (1)..(5): /note=“Variant residues given in the sequence have no preference with respect to those in the annotations for variant positions”
  source: /note=“See specification as filed for detailed description of substitutions and preferred embodiments”
KYVMH

SEQ ID NO: 73 - protein, 17 aa, Artificial Sequence
  source: /note=“Description of Artificial Sequence: Synthetic peptide”
  VARIANT (8)..(8): /replace=“Ile”
  VARIANT (14)..(14): /replace=“Thr”
  SITE (1)..(17): /note=“Variant residues given in the sequence have no preference with respect to those in the annotations for variant positions”
  source: /note=“See specification as filed for detailed description of substitutions and preferred embodiments”
SIYPSGGFTFYADSVKG

SEQ ID NO: 74 - protein, 11 aa, Artificial Sequence
  source: /note=“Description of Artificial Sequence: Synthetic peptide”
  VARIANT (10)..(10): /replace=“Asp”
  SITE (1)..(11): /note=“Variant residues given in the sequence have no preference with respect to those in the annotations for variant positions”
  source: /note=“See specification as filed for detailed description of substitutions and preferred embodiments”
IKLGTVTTVEY

SEQ ID NO: 75 - protein, 14 aa, Artificial Sequence
  source: /note=“Description of Artificial Sequence: Synthetic peptide”
  VARIANT (4)..(4): /replace=“Ser”
  VARIANT (5)..(5): /replace=“Arg” or “Ser”
  VARIANT (9)..(9): /replace=“Gly”
  SITE (1)..(14): /note=“Variant residues given in the sequence have no preference with respect to those in the annotations for variant positions”
  source: /note=“See specification as filed for detailed description of substitutions and preferred embodiments”
TGTNTDVGAYNYVS

SEQ ID NO: 76 - protein, 7 aa, Artificial Sequence
  source: /note=“Description of Artificial Sequence: Synthetic peptide”
  VARIANT (1)..(1): /replace=“Asp”
  VARIANT (3)..(3): /replace=“Asn” or “Ser”
  VARIANT (4)..(4): /replace=“His” or “Asn”
  SITE (1)..(7): /note=“Variant residues given in the sequence have no preference with respect to those in the annotations for variant positions”
  source: /note=“See specification as filed for detailed description of substitutions and preferred embodiments”
EVIDRPS

SEQ ID NO: 77 - protein, 10 aa, Artificial Sequence
  source: /note=“Description of Artificial Sequence: Synthetic peptide”
  VARIANT (3)..(3): /replace=“Tyr”
  VARIANT (5)..(5): /replace=“Ser”
  VARIANT (6)..(6): /replace=“Thr” or “Ser”
  VARIANT (7)..(7): /replace=“Ser”
  VARIANT (8)..(8): /replace=“Thr”
  SITE (1)..(10): /note=“Variant residues given in the sequence have no preference with respect to those in the annotations for variant positions”
  source: /note=“See specification as filed for detailed description of substitutions and preferred embodiments”
SSFTNRGIRV

SEQ ID NO: 78 - protein, 5 aa, Artificial Sequence
  source: /note=“Description of Artificial Sequence: Synthetic peptide”
SYIMM

SEQ ID NO: 79 - protein, 17 aa, Artificial Sequence
  source: /note=“Description of Artificial Sequence: Synthetic peptide”
SIYPSGGITFYADTVKG

SEQ ID NO: 80 - protein, 11 aa, Artificial Sequence
  source: /note=“Description of Artificial Sequence: Synthetic peptide”
IKLGTVTTVDY

SEQ ID NO: 81 - protein, 14 aa, Artificial Sequence
  source: /note=“Description of Artificial Sequence: Synthetic peptide”
TGTSSDVGGYNYVS

SEQ ID NO: 82 - protein, 7 aa, Artificial Sequence
  source: /note=“Description of Artificial Sequence: Synthetic peptide”
DVSNRPS

SEQ ID NO: 83 - protein, 10 aa, Artificial Sequence
  source: /note=“Description of Artificial Sequence: Synthetic peptide”
SSYTSSSTRV

SEQ ID NO: 84 - protein, 5 aa, Artificial Sequence
  source: /note=“Description of Artificial Sequence: Synthetic peptide”
MYMMM

SEQ ID NO: 85 - protein, 17 aa, Artificial Sequence
  source: /note=“Description of Artificial Sequence: Synthetic peptide”
SIYPSGGITFYADSVKG

SEQ ID NO: 86 - protein, 14 aa, Artificial Sequence
  source: /note=“Description of Artificial Sequence: Synthetic peptide”
TGTSSDVGAYNYVS

SEQ ID NO: 87 - protein, 119 aa, Artificial Sequence
  source: /note=“Description of Artificial Sequence: Synthetic polypeptide”
EVQLLESGGGLVQPGGSLRLSCAASGFTFSSYIMMVWRQAPGKGLEWVSSIYPSGGITFYADWKGRFTISRDNSKNTLYLQMNSLRAEDTAVYYCARIKLGTVTTVDYWGQGTLVTVSS

SEQ ID NO: 88 - protein, 110 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 89 - protein, 120 aa, Artificial Sequence
  source: /note=“Description of Artificial Sequence: Synthetic polypeptide”
EVQLLESGGGLVQPGGSLRLSCAASGFTFSMYMMMWVRQAPGKGLEVWSSIYPSGGITFYADSVKGRFTISRDNSKNTLYLQMNSLRAEDTAIYYCARIKLGTVTTVDYWGQGTLVTVSS

SEQ ID NO: 90 - protein, 110 aa, Artificial Sequence
  source: /note=“Description of Artificial Sequence: Synthetic polypeptide”
QSALTQPASVSGSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVL

SEQ ID NO: 91 - protein, 216 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 92 - protein, 450 aa, Artificial Sequence
  source: /note=“Description of Artificial Sequence: Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 607 aa, Artificial Sequence
  source: /note=“Description of Artificial Sequence: Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 94 - DNA, 711 bp, Artificial Sequence
  source: /note=“Description of Artificial Sequence: Synthetic polynucleotide”
atgagggccctgctggctagactgctgctgtgcgtgctggtcgtgtccgacagcaagggccagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggccagcccaaggccaacccaaccgtgacactgttccccccatcctccgaggaactgcaggccaacaaggccaccctggtctgcctgatctcagatttctatccaggcgccgtgaccgtggcctggaaggctgatggctccccagtgaaggccggcgtggaaaccaccaagccctccaagcagtccaacaacaaatacgccgcctcctcctacctgtccctgacccccgagcagtggaagtcccaccggtcctacagctgccaggtcacacacgagggctccaccgtggaaaagaccgtcgcccccaccgagtgctcatga

SEQ ID NO: 95 - DNA, 1887 bp, Artificial Sequence
  source: /note=“Description of Artificial Sequence: Synthetic polynucleotide”
atggaaacagacaccctgctgctgtgggtgctgctgctgtgggtgcccggctccacaggcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgctggcggcggaggaagcggaggaggtggcagcggtggcggtggctccggcggaggtggctccggaatccctccccacgtgcagaagtccgtgaacaacgacatgatcgtgaccgacaacaacggcgccgtgaagttccctcagctgtgcaagttctgcgacgtgaggttcagcacctgcgacaaccagaagtcctgcatgagcaactgcagcatcacaagcatctgcgagaagccccaggaggtgtgtgtggccgtgtggaggaagaacgacgaaaacatcaccctcgagaccgtgtgccatgaccccaagctgccctaccacgacttcatcctggaagacgccgcctcccccaagtgcatcatgaaggagaagaagaagcccggcgagaccttcttcatgtgcagctgcagcagcgacgagtgcaatgacaacatcatctttagcgaggagtacaacaccagcaaccccgactgataa

SEQ ID NO: 96 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 97 - protein, 21 aa, Artificial Sequence
  source: /note=“Description of Artificial Sequence: Synthetic peptide”
GGGGSGGGGSGGGGSGGGGSG

SEQ ID NO: 98 - protein, 4 aa, Artificial Sequence
  source: /note=“Description of Artificial Sequence: Synthetic peptide”
QFNS

SEQ ID NO: 99 - protein, 4 aa, Artificial Sequence
  source: /note=“Description of Artificial Sequence: Synthetic peptide”
QAQS

SEQ ID NO: 100 - protein, 6 aa, Artificial Sequence
  source: /note=“Description of Artificial Sequence: Synthetic peptide”
PKSCDK

SEQ ID NO: 101 - protein, 6 aa, Artificial Sequence
  source: /note=“Description of Artificial Sequence: Synthetic peptide”
PKSSDK

SEQ ID NO: 102 - protein, 4 aa, Artificial Sequence
  source: /note=“Description of Artificial Sequence: Synthetic peptide”
LSLS

SEQ ID NO: 103 - protein, 4 aa, Artificial Sequence
  source: /note=“Description of Artificial Sequence: Synthetic peptide”
ATAT

SEQ ID NO: 104 - protein, 117 aa, Homo sapiens
GAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 105 - protein, 115 aa, Homo sapiens
VKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 106 - protein, 122 aa, Homo sapiens [sequence omitted]

SEQ ID NO: 107 - protein, 110 aa, Homo sapiens
LCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 108 - protein, 122 aa, Artificial Sequence
  source: /note=“Description of Artificial Sequence: Synthetic polypeptide”
VTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 109 - protein, 118 aa, Artificial Sequence
  source: /note=“Description of Artificial Sequence: Synthetic polypeptide”
QVQLQESGPGLVKPSQTLSLTCTVSGGSISNDYWTWIRQHPGKGLEYIGYISYTGSTYYNPSLKSRVTISRDTSKNQFSLKLSSVTAADTAVYYCARSGGWLAPFDYWGRGTLVTVSS

SEQ ID NO: 110 - protein, 113 aa, Artificial Sequence
  source: /note=“Description of Artificial Sequence: Synthetic polypeptide”
DIVMTQSPDSLAVSLGERATINCKSSQSLFYHSNQKHSLAWYQQKPGQPPKLLIYGASTRESGVPDRFSGSGSGTDFTLTISSLQAEDVAVYYCQQYYGYPYTFGGGTKVEIK

SEQ ID NO: 111 - protein, 119 aa, Artificial Sequence
  source: /note=“Description of Artificial Sequence: Synthetic polypeptide”
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSS

SEQ ID NO: 112 - protein, 111 aa, Artificial Sequence
  source: /note=“Description of Artificial Sequence: Synthetic polypeptide”
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIK

SEQ ID NO: 113 - protein, 445 aa, Artificial Sequence
  source: /note=“Description of Artificial Sequence: Synthetic polypeptide”
QVQLQESGPGLVKPSQTLSLTCTVSGGSISNDYWTWIRQHPGKGLEYIGYISYTGSTYYNPSLKSRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 220 aa, Artificial Sequence
  source: /note=“Description of Artificial Sequence: Synthetic polypeptide”
DIVMTQSPDSLAVSLGERATINCKSSQSLFYHSNQKHSLAWYQQKPGQPPKLLIYGASTRESGVPDRFSGSGSGTDFTLTISSLQAEDVAVYYCQQYYGYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 446 aa, Artificial Sequence
  source: /note=“Description of Artificial Sequence: Synthetic polypeptide”
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGA

SEQ ID NO: 116 - protein, 218 aa, Artificial Sequence
  source: /note=“Description of Artificial Sequence: Synthetic polypeptide”
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 26 aa, Artificial Sequence
  source: /note=“Description of Artificial Sequence: Synthetic peptide”
  SITE (1)..(25): /note=“This region may encompass 4-5 ′Gly Gly Gly Gly Ser′ repeating units”
GGGGSGGGGSGGGGSGGGGSGGGGSG

SEQ ID NO: 118 - protein, 108 aa, Artificial Sequence; identical to SEQ ID NO: 52
  source: /note=“Description of Artificial Sequence: Synthetic polypeptide”